(19) United States
(12) Patent Application Publication
(10) Pub. No.: US 2026/0256896 A1
(43) Pub. Date: Sep. 3, 2026

(54) HLA CLASS II DEFICIENT CELLS, HLA CLASS I DEFICIENT CELLS CAPABLE OF EXPRESSING HLA CLASS II PROTEINS, AND USES THEREOF

(71) Applicant: University of Washington through its Center for Commercialization, Seattle, WA (US)

(72) Inventors: David W. Russell, Seattle, WA (US); Roli K. Hirata, Seattle, WA (US)

(21) Appl. No.: 19/665,678

(22) Filed: May 1, 2026

Related U.S. Application Data
(62) Division of application No. 16/441,889, filed on Jun. 14, 2019, now Pat. No. 12,636,354.
Application No. 16/441,889 is a continuation of application No. 14/391,328, filed on Mar. 15, 2013, now abandoned.
(60) Provisional application No. 61/625,314, filed on Apr. 17, 2012.

(51) Int. Cl. A61K 39/00; A61K 35/545; A61K 40/10; A61K 40/22; A61K 40/41; A61K 48/00; C07K 14/47; C07K 14/705; C12N 15/90

(52) U.S. Cl. CPC A61K 39/001; A61K 35/545; A61K 40/10; A61K 40/22; A61K 40/416; A61K 40/418; C07K 14/4705; C07K 14/70539; C12N 15/907; A01K 2217/075; A61K 48/00; C12N 2799/025; C12N 2800/30

(57) ABSTRACT

The invention provides isolated primate cells preferably human cells that comprise a genetically engineered disruption in a human leukocyte antigen (HLA) class II-related gene, which results in deficiency in MHC class II expression and function. This invention also provides isolated cells further comprising a genetically engineered disruption in a beta-2 microglobulin (B2M) gene, which results in HLA class I/class II deficiency. Also provided are the method of using the cells for transplantation and treating a disease condition.

### SEQUENCE LISTING STATEMENT

A computer readable form of the Sequence Listing is filed with this application by electronic submission and is incorporated into this application by reference in its entirety. The Sequence Listing is contained in the file created on Sep. 20, 2025 having the file name “12-094-PCT.xml” and is 154,417 bytes in size.

### STATEMENT OF GOVERNMENT RIGHTS

This invention was made with government support under grant numbers R01GM086497 and R01DK55759 awarded by the National Institutes of Health. The government has certain rights in the invention.

### BACKGROUND OF THE INVENTION

Human pluripotent stem cells have the potential to treat diseases affecting almost every organ system. However, the clinical use of human pluripotent stem cells and their derivatives has a major limitation—rejection of transplanted cells by the recipient due to differences in the major histocompatibility complex.

The major histocompatibility complex (MHC) is a cell surface multi-component molecule found in all vertebrates that mediates interactions of leukocytes with other leukocytes or other cells. The MHC gene family is divided into three groups: class I, class II and class III. In humans, MHC is referred to as human leukocyte antigen (HLA). HLA class II molecules (HLA-II) are transmembrane protein found only on professional antigen-presenting cells (APCs) including macrophages, dendritic cells and B cells. In addition, solid organ may sometimes express HLA class II genes that participate in immune rejection. The HLA class I (HLA-I) protein is expressed on all nucleated cells and consists of an HLA class I heavy chain (or a chain) and β-2 microglobulin (B2M). HLA class I protein presents peptides on the cell surface to CD8+ cytotoxic T cells. Six HLA class I α chains have been identified to date, including three classical (HLA-A, HLA-B and HLA-C) and three non-classical (HLA-E, HLA-F and HLA-G) a chains. The specificity for peptide binding on the HLA class I molecule peptide binding cleft is determined by the α chain. Recognition by CD8+ T cells of the peptides presented by the HLA class I molecule mediates cellular immunity.

HLA class II molecules and class I molecules are both heterodimers. Class I molecules consist of an alpha chain (or heavy chain) and β-2 microglobuin (B2M), whereas the class II molecules consist of two homologous subunits: the alpha subunit and beta subunit.

HLA class II (HLA-II) molecules or proteins present on the cell surface peptide antigens from extracellular proteins including proteins of an extracellular pathogen, while HLA class I proteins present peptides from intracellular proteins or pathogens. Loaded HLA class II proteins on the cell surface interact with CD4+ helper T cells. The interaction leads to recruitment of phagocytes, local inflammation, and/or humoral responses through the activation of B cells. Several HLA class II gene loci have been identified to date, including HLA-DM (HLA-DMA and HLA-DMB that encode HLA-DM α chain and HLA-DM β chain, respectively), HLA-DO (HLA-DOA and HLA-DOB that encode HLA-DO α chain and HLA-DO β chain, respectively), HLA-DP (HLA-DPA and HLA-DPB that encode HLA-DP α chain and HLA-DP β chain, respectively), HLA-DQ (HLA-DQA and HLA-DQB that encode HLA-DQ α chain and HLA-DQ β chain, respectively), and HLA-DR (HLA-DRA and HLA-DRB that encode HLA-DR α chain and HLA-DR β chain, respectively).

The HLA class I and/or class II proteins from an allogeneic source constitutes a foreign antigen in the context of transplantation. The recognition of non-self HLA class I and/or class II proteins is a major hurdle in using pluripotent cells for transplantation or replacement therapies.

Thus, although individualized stem cell preparations or HLA-diverse stem cell banks may address the current problem of transplantation, they require that multiple cell lines be characterized, differentiated into therapeutic cell products, and approved for human administration. This time-consuming, technically difficult, and expensive process is a major factor preventing stem cell-based therapies from entering clinical trials. Thus, there exists a need for a more effective and less expensive cell-based therapies that are not impeded by rejection.

### SUMMARY OF THE INVENTION

In a first aspect, the present invention provides isolated cells comprising a genetically engineered disruption in a human leukocyte antigen (HLA) class II-related gene, wherein the cell is a primate cell. In one embodiment, the HLA class II-related gene is selected from the group consisting of regulatory factor X-associated ankyrin-containing protein (RFXANK), regulatory factor 5 (RFX5), regulatory factor X-associated protein (RFXAP), class 11 transactivator (CIITA), HLA-DPA (α chain), HLA-DPB (β chain), HLA-DQA, HLA-DQB, HLA-DRA, HLA-DRB, HLA-DMA, HLA-DMB, HLA-DOA and HLA-DOB. In another embodiment, the cell comprises genetically engineered disruptions in at least two, at least three, or in all four of the HLA class II-related genes. In a further embodiment, the HLA class II-related gene is regulatory factor X-associated ankyrin-containing protein (RFXANK). In a still further embodiment, the cell comprises genetically engineered disruptions in all copies of the HLA class II-related gene. In another embodiment the cell further comprises one or more recombinant immunomodulatory genes, each capable of expressing an immunomodulatory polypeptide in the human cell. In a further embodiment, the one or more immunomodulatory genes comprise a polynucleotide capable of encoding an HLA II protein. In another embodiment, the one or more immunomodulatory genes comprise a polynucleotide capable of encoding a single chain fusion HLA class II protein. In a further embodiment, the cell further comprises a genetically engineered disruption in the β2-microglobulin (B2M) gene.

In another aspect, the present invention provides isolated cells comprising (a) a genetically engineered disruption in a beta-2 microglobulin (B2M) gene; and (b) one or more polynucleotides capable of encoding an HLA class II protein, or a single chain fusion HLA class II protein; wherein the cell is a primate cell.

In one embodiment of either of these aspects, the cell comprises genetically engineered disruptions of all copies of the B2M gene. In a further embodiment, the HLA II gene encodes an HLA protein selected from the group consisting of an HLA-DM α chain, an HLA-DM β chain, an HLA-DO α chain, an HLA-DO β chain, an HLA-DP α chain, an HLA-DP β chain, an HLA-DQ α chain, an HLA-DQ β chain, an HLA-DR α chain and an HLA-DR β chain.

In another embodiment, the single chain fusion HLA class II protein comprises at least a portion of an HLA class II gene α chain covalently linked to at least a portion of an HLA class II gene β chain, wherein the HLA class II gene is selected from the group consisting of HLA-DP, HLA-DQ, HLA-DR, HLA-DM, and HLA-DO. In a further embodiment, the single chain fusion HLA class II protein comprises a plurality of different single chain fusion HLA class II proteins. In another embodiment, the single chain fusion HLA class II protein comprises at least a portion of HLA-DQ α chain and at least a portion of HLA-DQ β chain. In a still further embodiment, the single chain fusion HLA class II protein comprises at least a portion of HLA-DQ α chain allele HLA-DQA1*01 and at least a portion of HLA-DQ β chain allele HLA-DQB1*02.

In a further embodiment of either aspect of the cells of the invention, the HLA protein or the single chain fusion HLA class II protein presents a first target peptide antigen on the cell surface. In one such embodiment, the first target peptide antigen is covalently linked to the single chain fusion HLA class II protein.

In another embodiment the cell further comprises a polynucleotide capable of encoding a single chain fusion HLA class I protein. In one such embodiment, the single chain fusion HLA class I protein comprises at least a portion of B2M covalently linked to at least a portion of an HLA class I α chain selected from the group consisting of HLA-A, HLA-B, HLA-C, HLA-E, HLA-F and HLA-G. In another such embodiment, the single chain fusion HLA class I protein comprises at least a portion of B2M covalently linked to at least a portion of HLA-A. In a further embodiment the single chain fusion HLA class I protein comprises at least a portion of B2M covalently linked to at least a portion of HLA-A0201. In another embodiment the cell further expresses a second target peptide antigen that is presented by the single chain fusion HLA class I protein on the cell surface. For example, the second target peptide antigen may be covalently linked to the single chain fusion HLA class I protein.

In another embodiment of either aspect of the cells of the invention, the cell further comprises one or more recombinant genes capable of encoding a suicide gene product. For example, the suicide gene product may comprise a protein selected from the group consisting of thymidine kinase and an apoptotic signaling protein.

The cells of either aspect of the may have a normal karyotype and may be non-transformed cells. The cells may be stem cells, such as hematopoietic stem cells, embryonic stem cells, pluripotent stem cells, induced pluripotent stem cells, liver stem cells, neural stem cells, pancreatic stem cells or mesenchymal stem cells. The stem cell may be differentiated, such as dendritic cells, pancreatic islet cells, liver cells, muscle cells, keratinocytes, neuronal cells, hematopoietic cells, lymphocytes, red blood cells, platelets, skeletal muscle cells, ocular cells, mesenchymal cells, fibroblasts, lung cells, gastrointestinal (GI) tract cells, vascular cells, endocrine cells, adipocytes or cardiomyocytes. The cells may be human cells.

In another aspect, the present invention provides vaccines comprising the cell of any one embodiment or combinations of embodiments of the cells of the present invention that include at least one target peptide antigen on the cell surface, wherein the vaccine is capable of eliciting in a primate an immune response specific for the target peptide antigen(s).

In a further aspect, the invention provides methods of transplantation in a patient in need thereof comprising the step of administering to the patient an effective amount of the cell or vaccine of any embodiment or combination of embodiments of the cells of the invention. In one such embodiment, the patient may be immune competent. In another embodiment, the cell or vaccine may comprise a differentiated cell.

### DESCRIPTION OF THE FIGURES

FIG. 1 shows the structure of exemplary two adeno-associated virus (AAV) gene targeting vectors, designed to insert either a TKNeo (AAV-RFXANK-ETKNpA) or HyTK (AAV-RFXANK-HyTK) gene controlled by an EF1alpha promoter (EF) into exon 3 of the RFXANK gene, which is also shown below the vectors. Selection of vector-infected cells with G418 or hygromycin (Hygro) allows one to isolate cells targeted by the TKNeo or HyTK vectors respectively. Subsequent expression of Cre recombinase and selection with gancyclovir (GCV) then allows one to isolate clones that have removed the TKNeo or HyTK genes, leaving behind two inactivated RFXANK alleles with stop codons in all 3 reading frames, a loxP site, and a polyadenylation site (StopX3-loxP-pA). LoxP is the recombination site for Cre recombinase. ITR is a vector inverted terminal repeat. Similar vectors could be designed to target other genes.

FIG. 2 (A) Schematic depiction of targeting strategy for infection of human embryonic stem cells with construct AAV-RFXANK-ETKNpA. (B) Photograph of stained gel showing polymerase chain reaction (PCR) products obtained after infection of human embryonic stem cells with AAV-RFXANK-ETKNpA and PCR using a forward primer homologous to the neomycin sequence of the selection cassette and a reverse primer homologous to the RFXANK gene which was outside the targeting homology arm, as indicated by the arrows above.

### DETAILED DESCRIPTION OF THE INVENTION

All references cited are herein incorporated by reference in their entirety. Within this application, unless otherwise stated, the techniques utilized may be found in any of several well-known references such as: Molecular Cloning: A Laboratory Manual (Sambrook, et al., 1989, Cold Spring Harbor Laboratory Press), Gene Expression Technology (Methods in Enzymology, Vol. 185, edited by D. Goeddel, 1991. Academic Press, San Diego, CA), “Guide to Protein Purification” in Methods in Enzymology (M. P. Deutshcer, ed., (1990) Academic Press, Inc.); PCR Protocols: A Guide to Methods and Applications (Innis, et al. 1990. Academic Press, San Diego, CA), Culture of Animal Cells: A Manual of Basic Technique, 2^{nd} Ed. (R. I. Freshney. 1987. Liss, Inc. New York, NY), Gene Transfer and Expression Protocols, pp. 109-128, ed. E. J. Murray, The Humana Press Inc., Clifton, N.J.), and the Ambion 1998 Catalog (Ambion, Austin, TX).

As used herein, the singular forms “a”, “an” and “the” include plural referents unless the context clearly dictates otherwise. “And” as used herein is interchangeably used with “or” unless expressly stated otherwise.

All embodiments of any aspect of the invention can be used in combination, unless the context clearly dictates otherwise.

In a first aspect, the present invention provides isolated cells comprising a genetically engineered disruption in a human leukocyte antigen (HLA) class II-related gene, wherein the cell is a primate cell. The HLA class II related genes as used in the instant application broadly refer to genes that encode proteins involved in the HLA class II mediated immune responses. Thus, HLA class II related genes encompass genes that encode the HLA class II molecules such as HLA-DM (SEQ ID NO: 48, 50), HLA-DO (SEQ ID NO: 52, 54), HLA-DP (SEQ ID NO: 36, 38), HLA-DQ (SEQ ID NO: 40, 42), and HLA-DR (SEQ ID NO: 44, 46). The sequences of exemplary HLA class II genes/proteins can be found in publicly available database under GenBank or IMGT/HLA database numbers NM_033554.3 (SEQ ID NOs:36, 37) for HLA-DPA, HLA00514 (SEQ ID NOs:38, 39) for HLA-DPB, HLA00601 (SEQ ID NOs:40, 41) for HLA-DQA, HLA00622 (SEQ ID NOs:42, 43) for HLA-DQB, NM_019111 (SEQ ID NOs:44, 45) for HLA-DRA, HLA00664 (SEQ ID NOs:46, 47) for HLA-DRB, NM_006120 (SEQ ID NOs:48, 49) for HLA-DMA, NM_002118 (SEQ ID NOs:50, 51) for HLA-DMB, NM_002119 (SEQ ID NOs:52, 53) for HLA-DOA, and NM_002120 (SEQ ID NOs:54, 55) for HLA-DOB.

In addition, HLA class II related genes also include genes that encode HLA class II regulatory proteins that regulate the expression of HLA class II molecules, including without limitation regulatory factor X-associated ankyrin-containing protein (RFXANK), regulatory factor 5 (RFX5), regulatory factor X-associated protein (RFXAP), and class II transactivator (CIITA). For example, regulatory factor X-associated ankyrin-containing protein (RFXANK) together with regulatory factor X-associated protein and regulatory factor-5 form a complex that binds to the X box motif of HLA class II gene promoters and activates transcription of the HLA class II genes. The sequences of exemplary HLA class II-related genes that regulate the expression of HLA class II molecules can be found in publicly available database under GenBank Accession Numbers NM_134440.1 (SEQ ID NOs:24, 25) and NM_003721.2 (SEQ ID NOs:26, 27) for RFXANK, NM_000449.3 (SEQ ID NOs:28, 29) and NM_001025603.1 (SEQ ID NOs:30, 31) for RFX5, NM_000538.3 (SEQ ID NOs:32, 33) for RFXAP and NM_000246.3 (SEQ ID NOs:34, 35) for CIITA. All the sequences disclosed under these GenBank Accession Numbers are herein incorporated by reference.

In certain embodiments, the invention provides an isolated primate preferably human cell that comprises a genetically engineered disruption in at least one HLA class II-related gene as defined herein. In certain particular embodiments, the cell comprises genetically engineered disruptions of all copies of the at least one HLA class II-related gene. In certain other embodiments, the cell comprises a plurality of genetically engineered disruptions in a plurality of HLA class II-related genes.

In certain embodiments, the HLA class II-related gene is selected from the group consisting of regulatory factor X-associated ankyrin-containing protein (RFXANK), regulatory factor 5 (RFX5), regulatory factor X-associated protein (RFXAP), and class II transactivator (CIITA). In certain particular embodiments, the cell comprises at least one genetically engineered disruption in at least one, at least two, at least three, or all of the HLA class II-related genes selected from the group consisting of regulatory factor X-associated ankyrin-containing protein (RFXANK), regulatory factor 5 (RFX5), regulatory factor X-associated protein (RFXAP), and class II transactivator (CIITA). Any combinations of these four HLA class II-related genes as target for genetic disruption to create HLA class II deficient cell are within the scope of the invention.

In certain other embodiments, the cell comprises at least one genetically engineered disruption of an HLA class II-related genes selected from the group consisting of regulatory factor X-associated ankyrin-containing protein (RFXANK)(SEQ ID NO: 24-27), regulatory factor 5 (RFX5) (SEQ ID NO: 28-31), regulatory factor X-associated protein (RFXAP) (SEQ ID NO: 32, 33), class II transactivator (CIITA) (SEQ ID NO: 34, 35), HLA-DPA (α chain)(SEQ ID NO: 36, 37), HLA-DPB (β chain) (SEQ ID NO: 38, 39), HLA-DQA (SEQ ID NO: 40, 41), HLA-DQB (SEQ ID NO: 42, 43), HLA-DRA (SEQ ID NO: 44, 45), HLA-DRB (SEQ ID NO: 46-47), HLA-DMA (SEQ ID NO: 48, 49), HLA-DMB (SEQ ID NO: 50, 51), HLA-DOA (SEQ ID NO: 52, 53) and HLA-DOB (SEQ ID NO: 54, 55).

The genetically engineered disruptions include without limitation deletions, insertions, substitutions and truncations of a target HLA class II-related gene that result in no expression of the target gene or expression of a truncated or mutated protein with no function or much reduced function as compared to the wild type protein. In certain embodiments, the genetically engineered disruption of a HLA class II-related gene leads to the expression of a truncated HLA class II-related protein. In certain particular embodiments, the HLA class II-related gene is RFXANK (SEQ ID NO: 24-27). In certain other particular embodiments, the HLA-related gene is selected from the group consisting of HLA-DPA (α chain)(SEQ ID NO: 36, 37), HLA-DPB (β chain) (SEQ ID NO: 38, 39), HLA-DQA (SEQ ID NO: 40, 41), HLA-DQB (SEQ ID NO: 42, 43), HLA-DRA (SEQ ID NO: 44, 45), HLA-DRB (SEQ ID NO: 46-47), HLA-DMA (SEQ ID NO: 48, 49), HLA-DMB (SEQ ID NO: 50, 51), HLA-DOA (SEQ ID NO: 52, 53) and HLA-DOB (SEQ ID NO: 54, 55). In certain further embodiments the cell comprises genetically engineered disruptions in all copies of the HLA class II-related gene.

In another aspect, the invention provides HLA class I and HLA class II deficient cells. In certain embodiments, the invention provides a primate cell, preferably a human cell that comprises a genetically engineered disruption in an HLA class II-related gene and further comprises a genetically engineered disruption in the β2-microglobulin (B2M) gene (SEQ ID NO: 1). In other particular embodiments, the cell further comprises genetically engineered disruptions of all copies of the B2M gene (SEQ ID NO: 1). In certain embodiments, the genetic disruptions in the B2M (SEQ ID NO: 1) gene result in defective or no expression of the B2M protein (SEQ ID NO: 2). In other particular embodiments, the cell further comprises genetically engineered disruptions of all copies of the B2M gene. In certain embodiments, the genetic disruptions in the B2M gene result in defective or no expression of the B2M protein. Since B2M is a common component of all HLA class I proteins, the disruptions preclude the expression of all natural HLA class I proteins on the cell surface. Thus, in this aspect of the invention an HLA class I/class II deficient cell is provided. The B2M coding sequence is shown in SEQ ID NO:1 (GenBank Accession Number NM_004048) and the B2M protein sequence is shown in SEQ ID NO:2. There may be many single nucleotide polymorphisms (SNPs) in the gene; as will be understood by those of skill in the art, the human cells and methods of the invention are applicable to any such B2M gene and SNPs.

Any suitable technique for introducing a genetically engineered disruption (in an HLA class II-related gene, in B2M gene or any other suitable gene) can be used; exemplary techniques for gene disruptions are disclosed throughout the application and are within the level of skill in the art based on the teachings herein and the teachings known in the art. Other exemplary techniques can be found, for example, in U.S. Patent Application Publication Number US2008/0219956, published Sep. 11, 2008, and incorporated by reference herein in its entirety. These techniques may optionally include steps to remove non-human DNA sequences from the cells after disruption of an HLA class II-related gene and optionally disruption of B2M gene.

One such techniques employs an adeno-associated virus gene targeting vector, optionally including removing the transgene used for targeting via techniques such as those described below, or by removing the transgene used for targeting by Cre-mediated loxP recombination, or other suitable recombination techniques. See Khan et al. 2011, Protocol, 6:482-501, which is incorporated by reference in its entirety. Exemplary targeting vectors and exemplary vector diagrams are also disclosed herein. It is within the level of those of skill in the art, based on the teachings herein and known in the art, to utilize a variety of techniques for making the HLA class II, preferably human cells, of the invention.

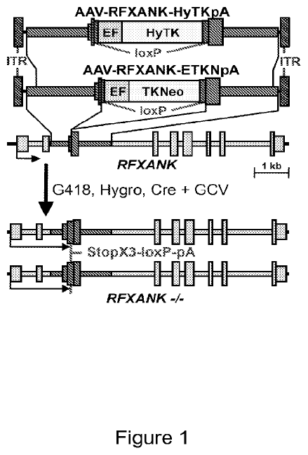

In certain embodiments, the cell genome of HLA class II deficient cells may comprise no more than 100, no more than 50 or no more than 30 nucleotides of non-human DNA sequences. In certain other embodiments, the cell genome may comprise 6, 5, 4, 3, 2, 1, or 0 nucleotides of non-human DNA sequences. Exemplary strategy for genetically disrupting the RFXANK gene is shown in FIG. 1. The non-human DNA sequences can be removed by a second round of targeting to delete the HyTK or TKNeo transgenes in the first vectors or by the Cre-mediated loxP recombination.

In certain other embodiments, the HLA class II or HLA class I/class II deficient cells further comprise one or more recombinant immunomodulatory genes. Suitable immunomodulatory genes include without limitation a gene encoding a viral protein that inhibits antigen presentation, a microRNA gene, a gene that encodes an HLA class II protein, or a gene that encodes a single chain (SC) fusion HLA class II protein. The term “single chain fusion HLA class II protein,” “single chain fusion HLA class II molecule” or “single chain fusion HLA class II antigen” refers to a fusion protein comprising at least a portion of the HLA class II α chain covalently linked, either directly or via a linker sequence, to at least a portion of an HLA class II chain or a class II α or β chain linked to a peptide antigen, or linked class II α and β chains also linked to a peptide antigen. On the other hand, the term “HLA class II protein,” “HLA class II molecule” or “HLA class II antigen” refers to a non-covalently associated heterodimer of an HLA class II α chain and an HLA β chain expressed on the surface of a wild type cell. In embodiments wherein the gene encodes an HLA class II protein (as opposed to a single chain fusion HLA class II protein), the gene is under control of a promoter not involved in normal class II expression in the cell. In one embodiment, the gene is episomally expressed; in another embodiment, the gene is integrated into the cell's genome. In either embodiment, the gene is operatively linked (i.e.: under transcriptional control) to a promoter not involved in normal class II expression in the cell. Any suitable promoter may be used, as may be determined by one of skill in the art based on the specific intended design and use of the constructs and cells.

In another aspect, the present invention provides isolated cells comprising (a) a genetically engineered disruption in a beta-2 microglobulin (B2M) gene; and (b) one or more polynucleotides capable of encoding an HLA II protein (alpha or beta chains) or a single chain fusion HLA class II protein; wherein the cell is a primate cell. Cells according to this aspect of the invention are HLA class I deficient cells

HLA class II deficient cells, HLA class I deficient cells, or HLA class I/class II deficient cells can be used as universal donor cells. In certain particular embodiments, the HLA class II deficient cells, HLA class I deficient cells, or HLA class I/class II deficient cells are hematopoietic cells or dendritic cells for use in transplantation. In addition, solid organ cells may sometimes express HLA class II genes that participate in immune rejection. Thus, in certain advantageous embodiments, the invention provides HLA class II, HLA class I deficient cells, or HLA class I/class II deficient cells for transplantation for treatment of diseases or injuries associated with solid organs.

In certain particular embodiments of any of the cells of the present invention, the HLA α and β chains are selected from the group consisting of α and β chains of HLA-DM, HLA-DR, HLA-DP, HLA-DQ, and HLA-DO. The α and β chains can be but do not have to be from the same HLA class II gene. For example, an HLA class II protein or a single chain fusion HLA class II protein may comprise at least a portion of an HLA-DQ α chain and at least a portion of an HLA-DQ β chain (also referred to as a dimeric construct). HLA class II proteins and single chain fusion HLA class II proteins comprising mismatching HLA class II alleles are also contemplated. In certain particular embodiments, an HLA class II protein or a single chain fusion HLA class II protein may comprise at least a portion of the HLA-DQ α chain allele HLA-DQA1*01 (SEQ ID NO:41) and at least a portion of the HLA-DQ β chain allele HLA-DQB1*02 (SEQ ID NO:43). In certain preferred embodiments, the leader sequence (or signal peptide) of the second portion of the fusion protein is removed in the fusion construct. For example, in a single chain fusion HLA class II protein that comprises at least a portion of HLA-DQA1*01 (SEQ ID NO: 41) at the N-terminus covalently linked to at least a portion of HLA-DQB1*02 (SEQ ID NO: 43) at the C-terminus, the HLA-DQA1*01 (SEQ ID NO: 41) leader sequence is left in, and the HLA-DQB1*02 (SEQ ID NO: 43) leader sequence is removed from the construct. In certain other embodiments, the cell further expresses at least two, at least three, or at least four or more different single chain fusion HLA class II proteins. In certain particular embodiments, the HLA class II protein or single chain fusion HLA class II protein also comprises a first target peptide antigen that occupies the peptide binding site of the HLA class II protein or single chain fusion HLA class II protein, wherein the peptide antigen is covalently linked to the HLA class II protein or single chain fusion HLA class II protein (also referred to as a trimeric construct). In certain other embodiments, the covalently linked peptide antigen is cleaved via a built-in protease cleavage site, and the cleaved peptide antigen can bind to the peptide binding site of the single chain fusion HLA-II protein for presentation.

Thus, HLA class II, HLA class I deficient cells, or HLA class I/class II deficient cells also encompass cells having genetically engineered disruptions in all copies of an HLA class II gene (e.g., disruptions in all copies of HLA-DQ α and/or β chain), wherein one HLA class II allele is genetically engineered to express, instead of the wild type HLA class II protein, an HLA class II protein of interest or a single chain fusion HLA class II protein (i.e., genetically targeted knockin in one HLA-II allele). Take HLA-DQ as an example, in certain embodiments, HLA-DQ^{-/-} cells express HLA-DQ protein only in the context of a single chain fusion HLA-DQ protein from an HLA-DQ genetic locus. In certain advantageous embodiments, the expression of the single chain fusion HLA class II protein is regulated by the endogenous HLA-DQ regulatory sequence located at the HLA-DQ locus.

In related embodiments, HLA class II, HLA class I deficient cells, or HLA class I/class II deficient cells further encompass cells having genetically engineered disruptions in all copies of a certain HLA-II gene, wherein all alleles of the specific HLA-II gene are genetically engineered to express, instead of the wild type HLA-II protein, single chain fusion HLA class II proteins (i.e., genetically targeted knockin in all HLA-II alleles). HLA class II, HLA class I deficient cells, or HLA class I/class II deficient cells with such genetic disruptions express a particular HLA-II protein only in the context of single chain fusion HLA class II proteins from the genetic loci of all the alleles of the particular HLA-II gene.

HLA class II proteins and single chain fusion HLA class II proteins comprising sequence variants and fragments of HLA class II α chains and β chains are contemplated by the instant invention, wherein such HLA class II proteins or single chain fusion constructs nevertheless possess normal HLA class II functions, e.g., forming proper secondary structure of the heterodimer on the cell surface, presenting peptides in the peptide binding site, interacting with CD4+ helper T cells and triggering HLA class II-mediated immune responses. In certain embodiments, the variants share at least 75%, 78%, 80%, 81%, 85%, 88%, 90%, 91%, 92%, 93%, 94%, 95%, 96%, 97%, 98%, 99%, or complete sequence homology with the naturally occurring HLA class II α or β chain sequences, wherein the variants possess normal HLA class II functions. In certain other embodiments, the variants share at least 75%, 80%, 81%, 85%, 88%, 90%, 91%, 92%, 93%, 94%, 95%, 96%, 97%, 98%, 99%, or complete sequence homology with the sequences of HLA class II α or β chains as shown in SEQ ID NOs:37, 39, 41, 43, 45, 47, 49, 51, 53 and 55.

Further, the HLA class II, HLA class I deficient cells, or HLA class I/class II deficient cells can be engineered to recombinantly express a single chain fusion HLA class I protein in a B2M−/− genetic background. The HLA class I deficient cells or HLA class I/class II deficient cells recombinantly expressing a single chain fusion HLA class I protein are nevertheless deficient in normal B2M function in that the cells do not express wild type B2M protein (SEQ ID NO:2) that can form a non-covalently associated heterodimer with any HLA class I α chain on the cell surface.

The term “single chain fusion HLA class I protein,” “single chain fusion HLA class I molecule” or “single chain fusion HLA class I antigen” refers to a fusion protein comprising at least a portion of the B2M protein covalently linked, either directly or via a linker sequence, to at least a portion of an HLA-I α chain. On the other hand, the term “HLA class I protein,” “HLA class I molecule” or “HLA class I antigen” refers to a non-covalently associated heterodimer of B2M and an HLA α chain expressed on the surface of a wild type cell.

As used herein, the term “HLA class I α chain” or “HLA-I heavy chain” refers to the α chain of the HLA class I heterodimer. HLA class I heavy chain includes without limitation HLA class I α chains HLA-A, HLA-B, HLA-C, HLA-E, HLA-F, and HLA-G. Representative DNA and protein sequences are provided for HLA-A (GenBank No. K02883.1, SEQ ID NO:3; UniProt No. P01892, SEQ ID NO:4), HLA-B (NM_005514, SEQ ID NO:5; NP_005505; SEQ ID NO:6), HLA-C(NM_002117, SEQ ID NO:7; NP_002108, SEQ ID NO:8), HLA-E (NM_005516, SEQ ID NO:9; NP_005507, SEQ ID NO:10), HLA-F (NM_018950, SEQ ID NO:11; NP_061823, SEQ ID NO:12), and HLA-G (NM_002127, SEQ ID NO:13; NP_002118, SEQ ID NO:14).

In addition, although the term “HLA class I or II protein/molecule” is known to refer to the MHC class I or II protein/molecule in human, the terms HLA and MHC are sometimes used interchangeably throughout this application: for example, the term HLA class I or HLA class II protein can also be used to refer to the primate equivalent to the HLA class I protein or HLA class II protein, respectively, in a primate. One of skill in the art will be able to discern the meaning of the term based on the content.

Thus, HLA class I deficient cells or HLA class I/class II deficient cells also encompass cells having genetically engineered disruptions in all copies of the B2M gene, wherein one B2M allele is genetically engineered to express, instead of the wild type B2M protein, a single chain fusion HLA class I protein (i.e., genetically targeted knockin in one B2M allele). B2M−/− cells with such genetic background express B2M only in the context of the single chain fusion HLA class I protein from a B2M genetic locus. In certain advantageous embodiments, the expression of the single chain fusion HLA class I protein is regulated by the endogenous B2M regulatory sequence located at the B2M locus.

In related embodiments, HLA class I deficient cells or HLA class I/class II deficient cells further encompass cells having genetically engineered disruptions in all copies of the B2M gene, wherein all B2M alleles are genetically engineered to express, instead of the wild type B2M protein, single chain fusion HLA class I proteins (i.e., genetically targeted knockin in all B2M alleles). HLA class I deficient cells or HLA class I/class II deficient cells with such genetic disruptions express B2M only in the context of single chain fusion HLA class I proteins from the genetic loci of all the alleles of the B2M gene. In certain embodiments, the cells are genetically engineered to express the same type of single chain fusion HLA class I protein from the genetic loci of all alleles of the B2M gene; while in other embodiments, the cells are genetically engineered to express different types of single chain fusion HLA class I proteins from different genetic loci of the B2M gene.

In certain embodiments, the single chain fusion HLA class I protein comprises at least a portion of B2M (SEQ ID NO: 2) and at least a portion of HLA-A (SEQ ID NO: 4), HLA-B (SEQ ID NO: 6), HLA-C(SEQ ID NO: 8), HLA-E (SEQ ID NO: 10), HLA-F (SEQ ID NO: 12) or HLA-G (SEQ ID NO: 14) (also referred to as a dimeric construct). In certain preferred embodiments, the HLA α chain contained in the single chain fusion HLA class I protein does not contain the leader sequence (or signal sequence) of the HLA class I α chain (leaderless HLA α chain). In certain other embodiments, the single chain fusion HLA class I protein comprises at least a portion of B2M (SEQ ID NO: 2) and at least a portion of HLA-C(SEQ ID NO: 8), HLA-E (SEQ ID NO: 10) or HLA-G (SEQ ID NO: 14). In certain further embodiments, the single chain fusion HLA class I protein comprises at least a portion of B2M (SEQ ID NO: 2) and at least a portion of HLA-A (SEQ ID NO: 4), HLA-E (SEQ ID NO: 10) or HLA-G (SEQ ID NO: 14). In certain preferred embodiments, the single chain fusion HLA class I protein comprises a leader sequence (or signal peptide) covalently linked to at least a portion of B2M and at least a portion of an HLA α chain to ensure proper folding of the single chain fusion on the cell surface. The leader sequence can be the leader sequence of the B2M protein, the leader sequence of an HLA α chain protein or the leader sequence of other secretary proteins. In certain particular embodiments, the single chain fusion HLA class I protein comprises a B2M protein with its leader sequence removed. In certain other particular embodiments, the single chain fusion HLA class I protein comprises an HLA α chain protein with its leader sequence removed. Certain HLA class I α chains are highly polymorphic. As will be understood by those of skill in the art, the human cells and methods of the invention are applicable to any such HLA α chains and polymorphism thereof.

Single chain fusion HLA class I proteins comprising sequence variants and fragments of B2M and/or HLA α chains are contemplated by the instant invention, wherein such single chain fusion constructs nevertheless possess normal HLA class I functions, e.g., forming proper secondary structure of the heterodimer on the cell surface, presenting peptides in the peptide binding cleft and engaging the inhibitory receptors on the surface of NK cells. In certain embodiments, the variants share at least 75%, 80%, 81%, 85%, 88%, 90%, 91%, 92%, 93%, 94%, 95%, 96%, 97%, 98%, 99%, or complete sequence homology with the naturally occurring HLA heavy chains and B2M sequences, wherein the variants possess normal HLA class I functions. In certain other embodiments, the variants share at least 75%, 80%, 81%, 85%, 88%, 90%, 91%, 92%, 93%, 94%, 95%, 96%, 97%, 98%, 99%, or complete sequence homology with the sequences of B2M or HLA heavy chains as shown in SEQ ID NOs:2, 4, 6, 8, 10, 12 or 14.

In certain particular embodiments, the HLA-A variants share at least 85%, 88,%, 90%, 91%, 92%, 93%, 94%, 95%, 96%, 97%, 98%, 99%, or complete sequence homology with SEQ ID NO:4. In certain other particular embodiments, the HLA-B variants share at least 81%, 83%, 85%, 88,%, 90%, 91%, 92%, 93%, 94%, 95%, 96%, 97%, 98%, 99%, or complete sequence homology with SEQ ID NO:6. In certain further embodiments, the HLA-C variants share at least 90%, 91%, 92%, 93%, 94%, 95%, 96%, 97%, 98%, 99%, or complete sequence homology with SEQ ID NO:8. In yet other embodiments, the HLA-E variants share at least 97%, 98%, 99%, or complete sequence homology with SEQ ID NO:10. In certain particular embodiments, the HLA-F variants share at least 99%, or complete sequence homology with SEQ ID NO:12. In certain other embodiments, the HLA-G variants share at least 98%, 99%, or complete sequence homology with SEQ ID NO:14.

In certain other embodiments, the single chain fusion HLA class I protein comprises full-length B2M (SEQ ID NO: 2) (including its leader sequence) and an HLA α chain without the leader sequence (leaderless HLA α chain); while in certain other embodiments, the single chain fusion HLA class I protein comprises B2M (SEQ ID NO: 2) protein without the leader sequence. It is understood that B2M−/− cells expressing two, three or more different types of single chain fusion HLA class I protein in any combination, for example, expressing SC fusion comprising HLA-A (SEQ ID NO: 4)(or a leaderless HLA-A) and SC fusion comprising HLA-C(SEQ ID NO: 8) (or a leaderless HLA-C), expressing SC fusion comprising HLA-A (SEQ ID NO: 4)(or a leaderless HLA-A) and SC fusion comprising HLA-E (SEQ ID NO: 10) (or a leaderless HLA-E), or expressing SC fusion comprising HLA-B (SEQ ID NO: 6)(or a leaderless HLA-B), SC fusion comprising HLA-E (SEQ ID NO: 10)(or a leaderless HLA-E) and SC fusion comprising HLA-G (SEQ ID NO: 14)(or a leaderless HLA-G), etc., are all contemplated by the invention and fall within the scope of the invention.

Natural killer (NK) cells are part of the innate immune response. Several pathogens can down regulate HLA class I protein expression in infected cells. The NK cells monitor infection by recognizing and inducing apoptosis in cells that do not express HLA class I proteins. The inhibitory receptors on the NK cell surface recognize HLA class I α chain alleles thereby preventing NK-medicated apoptosis in uninfected normal cells. Thus, in certain particular embodiments, the single chain fusion HLA-I protein inhibits NK cell-mediated killing of cells that do not express endogenous HLA class I proteins by binding to the inhibitory receptors on the NK cells. For example, HLA-E is a ligand for the CD94/NKG2 receptor of NK cells that inhibits NK cell-mediated apoptosis. Thus, in certain particular embodiments, the B2M−/− cell expresses the single chain fusion HLA class I protein comprising at least a portion of B2M and at least a portion of HLA-E. In addition, HLA-G is normally expressed on the surface of placental cytotrophoblasts that do not express HLA-A, B or C, and it protects these cells from NK cell-mediated lysis by interacting with the inhibitory ILT2(LIR1) receptor on NK cells (Pazmany et al., 1996, Science 274, 792-795). Thus, in certain other preferred embodiments, the B2M−/− cell expresses the single chain fusion HLA class I protein comprising at least a portion of B2M and at least a portion of HLA-G.

In certain particular embodiments, the single chain fusion HLA class I protein comprises at least a portion of B2M and at least a portion of HLA-A0201. HLA-A0201 (SEQ ID NO:4) is a common HLA class I allele found in a large percentage of the population in the United States. Thus, in certain advantageous embodiments, the isolated cell expresses the single chain fusion HLA class I protein comprising at least a portion of B2M and at least a portion of HLA-A0201 in a B2M−/− genetic background, wherein the isolated cell is immune compatible with a large percentage of the human population in the United States. Other suitable common alleles that can be used include without limitation HLA-A0101, HLA-A0301, HLA-B0702, HLA-B0801, HLA-C0401, HLA-C0701, and HLA-C0702. In certain preferred embodiments, the HLA allele comprises at least a portion of HLA-A0201 (SEQ ID NO:4), HLA-B0702 (SEQ ID NO:6) or HLA-C0401 (SEQ ID NO:8).

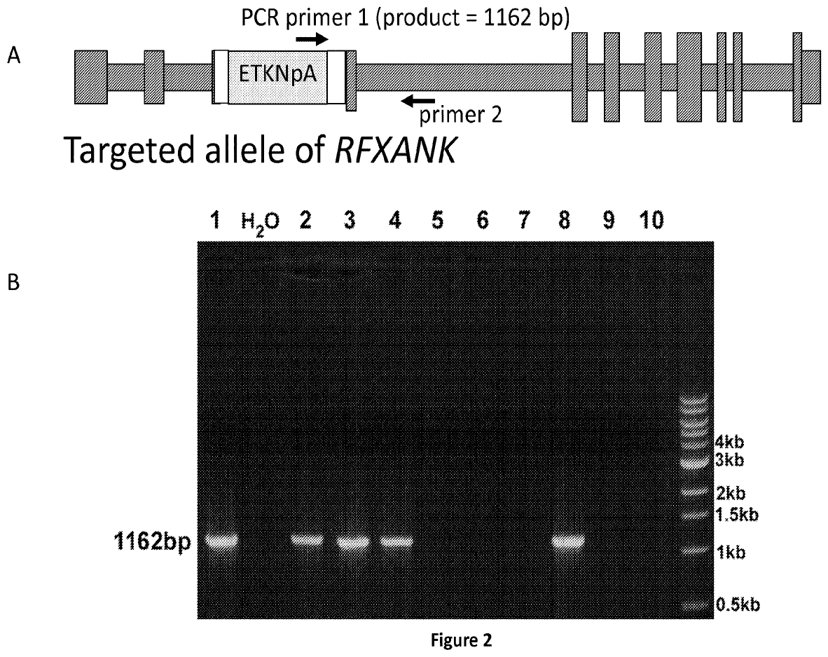

In certain further embodiments, the single chain fusion HLA class I protein also comprises a second target peptide antigen that occupies the peptide binding cleft of the single chain fusion HLA class I protein, wherein the peptide antigen is covalently linked to the single chain fusion HLA class I protein (also referred to as a trimeric construct). An example of the trimeric construct is shown in FIG. 2. The HLA-bGBE construct of FIG. 2 comprises B2M and HLA-E covalently linked to a peptide antigen (such as, but not limited to, the HLA-G signal peptide as illustrated in the figure) (SEQ ID NO:23) designed to occupy the peptide binding cleft of the single chain fusion HLA class I protein. In certain other embodiments, the covalently linked peptide antigen is cleaved via a built-in protease cleavage site, and the cleaved peptide antigen can bind to the peptide binding cleft of the single chain fusion HLA-I protein for presentation.

In certain alternative embodiments, the peptide antigen occupying the peptide binding cleft of the single chain fusion HLA class I protein is produced by the intracellular antigen processing pathway, in which the peptide antigen is produced by proteasome, transported to and loaded onto the single chain fusion HLA class I protein in the endoplasmic reticulum. In certain particular embodiments, the peptide antigen comprises a peptide of a tumor antigen. In certain other embodiments, the peptide antigen comprises a peptide of a protein from a pathogen including without limitation a bacterium, a virus, a fungus and a parasite. In further embodiments, the peptide antigen comprises a peptide of a tumor antigen. In certain particular embodiments, the HLA class I deficient cell or HLA class I/class II deficient cell expresses a single chain fusion HLA class I protein that is covalently linked to a peptide that does not comprise an auto-antigen or neo-antigen to the patient. It is within the ability of a skilled person to design the single chain fusion HLA class I protein and the peptide antigen presented thereon to modulate the immune response that may be elicited in a recipient.

The isolated HLA class II deficient cells, HLA class I deficient cells, or HLA class I/class II deficient cells expressing an HLA class II protein or a single chain fusion HLA class II protein optionally a single chain fusion HLA class I protein comprising a specific peptide antigen either covalently or non-covalently bound to the single chain fusion proteins can be used, for example, for administration to a recipient to elicit an immune response. Accordingly, in a related aspect, the invention provides a vaccine comprising the isolated cell of the invention, wherein the vaccine is capable of eliciting in a recipient an immune response specific for the target peptide antigen. The immune response includes without limitation a cellular immune response and/or a humoral immune response. The vaccine may comprise a stem cell or a differentiated cell; in certain particular embodiments, the cell is a differentiated dendritic cell. In certain other embodiments, the cell further expresses a cytokine. Any suitable cytokine can be used; in certain particular embodiments, the cytokine is IL2 or IFN-γ. In certain preferred embodiments, the cell is a human cell and the recipient is a human. Thus, in a further aspect, the invention provides kits comprising the vaccines of the invention and optionally an immune adjuvant.

The single chain fusion HLA class I protein, HLA class II protein, or single chain fusion HLA class II protein can be expressed from an expression vector that allows either transient or more preferably, stable expression of the protein in a cell. Exemplary suitable expression vectors are known in the art. One such example is a retroviral vector, which is capable of integrating into the cellular genome to provide long-term, stable expression of an exogenous gene. In certain particular embodiments, the viral vector is derived from human foamy virus, a type of retrovirus. Other suitable viral vectors include without limitation vectors derived from retrovirus, adenoviral virus, adeno-associated virus, lentivirus, herpes simplex virus, vaccinia virus, and pox virus.

In certain preferred embodiments, the polynucleotide capable of encoding a single chain fusion HLA class I or class II protein or an HLA class II protein is integrated into the chromosome of the cells, preferably into the B2M or the HLA class II loci, for stable expression. Thus, in certain preferred embodiments, the B2M loci are disrupted by inserting in the B2M loci the polynucleotide capable of encoding a single chain fusion HLA class I protein to replace the expression of the endogenous wild type B2M protein. Thus, in certain other preferred embodiments, certain HLA-II loci are disrupted by inserting in the HLA-II loci the polynucleotide capable of encoding an HLA class II protein or a single chain fusion HLA class II protein to replace the expression of the endogenous wild type HLA-II protein. The result of such gene targeting precludes formation of wild type HLA class I proteins and specific HLA class II proteins but permits expression of a predetermined HLA class II protein or single chain fusion HLA class I or class II protein of choice on the surface of the otherwise HLA class II deficient cells. Other expression vectors are also contemplated and the selection of suitable expression vector is within the ability of one ordinary skill in the art.

The “isolated cell” can be any suitable cell type for a given purpose. For example, the cell can be a pluripotent stem cell or a differentiated cell. “A stem cell” broadly encompasses any cells that are capable of further differentiation. “A pluripotent stem cell” refers to a stem cell that has the potential to differentiate into any of the three germ layers: endoderm, mesoderm or ectoderm. “An adult stem cell,” on the other hand, is multipotent in that it can produce only a limited number of cell types. “An embryonic stem (ES) cell” refers to a pluripotent stem cell derived from the inner cell mass of the blastocyst, an early-stage embryo. “Induced pluripotent stem cells (iPS cells)” are pluripotent stem cell artificially derived from a non-pluripotent cell, typically an adult somatic cell, by artificially inducing expression of certain genes.

In another aspect, the invention provides a method of transplantation in a patient in need thereof comprising the step of administering to the patient an effective amount of the cells of the invention for transplantation. Because the HLA class II deficient cells and/or HLA class I deficient cells do not express wild type HLA class II protein (and optionally also HLA class I protein) on the cell surface, the cells when administered to a patient elicit minimal or no immune responses in the patient. Thus, transplantation using the HLA class II deficient cells and/or HLA class I deficient cells limits the need for taking immune suppressant therapies. Thus, in certain preferred embodiments, the patient is immune competent. In certain other embodiments, the cell is an isogeneic cell; while in other embodiments, the cell is an allogeneic cell.

In certain further embodiments, the cells of the invention are pluripotent stem cells; while in other embodiments, the cells of the invention are differentiated cells. In certain preferred embodiments, the cell is a human cell and the patient is a human patient. In certain particular embodiments, the method of transplantation comprises administering to a human an effective amount of the pluripotent stem cells or differentiated cells. In certain preferred embodiments, the cells of the invention further express one or more engineered single chain fusion HLA class II proteins and optionally also a single chain fusion HLA class I protein. In certain other embodiments, the cells are able to escape NK cell-mediated killing and elicit minimal or no immune response in the recipient after transplantation.

Transplantation therapy, replacement therapy or regenerative therapy refers to therapies for a disease condition by administering to a patient cells or tissues to replenish or replace defective cellular functions in a target organ. In certain particular embodiments, the need for transplantation arises as a result of physical or pathological injuries to a tissue or organ. In certain other particular embodiments, the need for transplantation arises as a result of one or more genetic defect or mutation in the patient and the transplantation of the cells of the invention replenishes or replaces defective cellular functions in the patient without the need for gene therapy to correct the underlying genetic mutation of the patient. In certain further embodiments, the transplantation includes without limitation hematopoietic stem cell transplantation, or transplantation of cells that are incorporated into an organ such as liver, kidney, pancreas, lung, brain, muscle, heart, gastrointestinal tract, nervous system, skin, bones, bone marrow, fat, connective tissue, immune system, or blood vessels. In certain particular embodiments, the target organ is a solid organ.

In certain particular embodiments, the cells administered to the recipient may or may not be incorporated into an organ in need of such therapy. In certain embodiments, the cells of the invention are differentiated into the desired cell type, either before or after transplantation, and provide the necessary cellular function without itself being incorporated into the tissue at the site of transplantation. For example, in certain embodiments for treating diabetes, the cells of the invention either as pluripotent stem cells or differentiated pancreatic beta islet cells are transplanted to a diabetic patient. The transplanted cells need not reconstitute a functioning pancreas: they just need to secrete insulin in response to glucose levels. In certain particular embodiments, the cells are transplanted into an ectopic location and are not fully incorporated into the pancreas. Transplantation of pluripotent cells of the invention, differentiated cells of the invention, or a tissue differentiated and developed ex vivo from the cells of the invention are all contemplated by the invention. In certain preferred embodiments, the cell is a human cell and the patient is a human patient. In certain other preferred embodiments, the cells of the invention express one or more single chain fusion HLA class II proteins and optionally also single chain fusion HLA class I proteins.

In a further aspect, the invention provides a method of treating a disease condition in a patient in need thereof comprising the step of administering to the patient an effective amount of the cell of the invention to treat the disease condition, wherein the disease condition is diabetes, an autoimmune disease, cancer, infection, anemia, cytopenia, myocardial infarction, heart failure, skeletal or joint condition, osteogenesis imperfecta or burns. In certain particular embodiments, the disease condition results from pathological or physical injuries to a tissue or organ. In certain embodiments, the cells of the invention are stem cells; while in other embodiments, the cells of the invention are differentiated cells. In certain preferred embodiments, the cell is a human cell and the patient is a human patient. In certain particular embodiments, the human cell is a differentiated cell. Transplantation of a tissue developed ex vivo from the cells of the invention is also contemplated by the invention. In certain preferred embodiments, the cells of the invention further express one or more single chain fusion HLA class II proteins and optionally also one or more single chain fusion HLA class I proteins. In certain embodiments, the cell is an isogeneic cell; while in other embodiments, the cell is an allogeneic cell.

In certain particular embodiments, the cell is a differentiated cell including without limitation a dendritic cell, lymphocyte, red blood cell, platelet, hematopoietic cell, pancreatic islet cell, liver cell, muscle cell, keratinocyte, cardiomyocyte, neuronal cell, skeletal muscle cell, ocular cell, mesenchymal cell, fibroblast, lung cell, GI tract cell, vascular cell, endocrine cell and adipocyte. In certain other particular embodiments, the invention provides a method of treating a disease condition in a solid organ. In certain embodiments, the cells of the invention used in treating a disease condition express one or more single chain fusion HLA class I proteins and optionally also one or more single chain fusion HLA class I proteins.

“Treating” a patient having a disease or disorder means accomplishing one or more of the following: (a) reducing the severity of the disease; (b) arresting the development of the disease or disorder; (c) inhibiting worsening of the disease or disorder; (d) limiting or preventing recurrence of the disease or disorder in patients that have previously had the disease or disorder; (e) causing regression of the disease or disorder; (f) improving or eliminating the symptoms of the disease or disorder; and (f) improving survival. In certain preferred embodiments, the disease or disorder is a disease or disorder that can be treated by transplantation of tissues or cells.

The effective amount of the isolated cells of the invention for transplantation or for treating a disease condition depends on a number of factors, such as the type of tissue, the severity of the disease condition, the transplantation reaction, the reason for transplantation, and the age and general health of the patient. The effective amount can be determined by a skilled researcher or clinician by routine practice. Due to the reduced immunogenicity of the transplanted cells, relative large amount of cells can be tolerated by a patient to achieve the desired therapeutic effects. Alternatively, the cells can be repeatedly transplanted at intervals until a desired therapeutic effect is achieved.

The route for administration of the cells of the invention is not limited in any particular way. Exemplary delivery routes include without limitation intravenous, intramuscular, subdermal, intraperitoneal, transcutaneous, intracutaneous, and subcutaneous route. The cells of the present invention can also be administered topically by injection. For example, the cells can be injected into an injured joint, a fractured bone, an infarct site, an ischemic site or their periphery.

In certain particular embodiments, the cells are administered via a delivery device including without limitation a syringe. For example, the cells can be suspended in a solution or a pharmaceutical composition contained in such a delivery device. The “solution” or “pharmaceutical composition” comprises a physiological compatible buffer and optionally a pharmaceutically acceptable carrier or diluent in which the cells of the invention remain viable. The use of such carriers and diluents is well known in the art. The solution includes without limitation physiologically compatible buffers such as Hank's solution, Ringer' solution, or physiologically buffered saline. The cells can be kept in the solution or pharmaceutical composition for short term storage without losing viability. In certain particular embodiments, the cells are frozen for long term storage without losing viability according to cryopreservation methods well-known in the art.

Aqueous injection suspensions may contain substances which increase the viscosity of the suspension, such as sodium carboxymethyl cellulose, sorbitol, or dextran, but still fluid to the extent that can be easily delivered by syringe injection. The solution is preferably sterile, stable under the conditions of manufacture and storage and is free of microorganism contamination through the use of, for example, parabens, chlorobutanol, phenol, ascorbic acid, thimerosal, and the like. The cells contained in the solution can be stem cells or differentiated cells as described herein, in a pharmaceutically acceptable carrier or diluent and, as required, other ingredients indicated above.

The cells may be administered systemically (e.g., intravenously) or locally (e.g., directly into a myocardial defect under the guidance of echocardiogram, or by direct application to damaged tissues or organs accessible during open surgery). For injections, the cells may be in an injectable liquid suspension preparation or in a biocompatible medium which is injectable in liquid form and becomes semi-solid at the site of damaged tissue. A syringe, a controllable endoscopic delivery device or other similar devises can be used so long as the needle lumen is of sufficient diameter (e.g. at least 30 gauge or larger) to avoid physical damages to the cells during delivery.

In certain other embodiments, the cells can be transplanted via a solid support, e.g., a planar surface or three-dimensional matrix. The matrix or planar surface is surgically implanted into the appropriate site in a patient. For example, a patient needing a pancreatic graft can have differentiated cells on a solid support surgically implanted in the pancreas tissue. Exemplary solid support includes without limitation a patch, a gel matrix (such as GELFOAM® from Pharmacia-Upjohn), polyvinyl alcohol sponge (PVA)-collagen gel implants (such as IVALON, Unipoint Industries, High Point, NC) and other similar or equivalent devices. A variety of other encapsulation technologies can be used with the cells of the invention, for example, WO 91/10470; WO 91/10425; U.S. Pat. Nos. 5,837,234; 5,011,472; 4,892,538).

The cells of the invention can be differentiated into various cell types of all three lineages, including without limitation hematopoietic, mesenchymal, pancreatic endoderm, cardiac and keratinocytes cells. In certain embodiments, the differentiated cell further expresses an HLA class II protein or a single chain fusion HLA class II protein and optionally also a single chain fusion HLA class I protein. In general, each cell type can be analyzed for HLA class II and optionally also class I protein expression, reactivity with human T cells and NK cells, appropriate differentiation markers, and xenotransplantation in immunodeficient mice to examine in vivo developmental potential. A brief discussion of each differentiated cell type follows.

In certain embodiments, the cells of the invention can be differentiated to hematopoietic cells for treating various hematopoietic diseases currently treated by bone marrow transplantation. Patients receiving transfusion can become refractory to platelet transfusions due to HLA mismatches. Anemic or cytopenic patients can be treated by delivering the cells of the invention-derived erythrocytes, platelets or neutrophils to treat bleeding or infection.

Further, stem cells of the invention-derived dendritic cells are antigen-presenting cells that can be used as cellular vaccines when properly engineered. In certain embodiments, the cells of the invention engineered to express an HLA class II protein or a single chain fusion HLA class II protein and optionally also a single chain fusion HLA class I protein and a unique peptide antigen are used to vaccinate against specific pathogen or tumor antigens. In certain other embodiments, differentiated HLA class II deficient, HLA class I deficient, or HLA class I/class II deficient cytotoxic lymphocytes with HLA-restricted reactivity against specific antigens are used to eliminate infected cells or tumor cells.

To obtain hematopoietic cells, the pluripotent cells are first allowed to form embryoid bodies, thereafter non-adherent cells were cultured in the presence of hematopoietic cytokines to develop into specific cell lineages. The differentiation of hematopoietic cells from the cells of the invention that express an HLA class II protein or a single chain fusion HLA class II protein and optionally also a single chain fusion HLA class I protein can be analyzed by flow cytometry and colony assays. The different cell populations are sorted based on their surface markers, and used to monitor the expression of HLA genes and reactivity with human NK cells and T cells as measured by Elispot, mixed lymphocyte reactions, and cytotoxicity assays. The effectiveness of the single chain fusion HLA constructs on suppression of NK cell-mediated killing can be examined at different stages of differentiation and transplantation. See Bix et al., 1991, Nature 349, 329-331. The hematopoietic stem cells can also be assayed using xenotransplantation models in, for example, immunodeficient mice (SCID-repopulating cells or SRCs).

The cells of the invention can be differentiated into hematopoietic cell either before or after the cells are administered to a patient. In certain preferred embodiments, the cell is a human cell and the patient is a human. In vitro hematopoietic differentiation can be performed according to established protocols. See for example, Slukvin et al., 2006, J Immunol 176:2924-32, and Chang et al., 2006, Blood 108:1515-23.

In certain other embodiments, the cells of the invention can be differentiated into mesenchymal stem cells. In certain embodiments, the cells of the invention express one or more HLA class II protein or single chain fusion HLA class II proteins and optionally also one or more single chain fusion HLA class I proteins. MSCs have the potential to form several differentiated cell types, including marrow stromal cells, adipocytes, osteoblasts, and chondrocytes. Thus, inducing pluripotent stem cells to form MSCs (iMSCs) is useful in treating skeletal and joint conditions. The iMSCs can be further differentiated into osteoblasts and formed bone in vivo. Deyle et al., 2012, Mol Ther. 20(1):204-13. Cellular responses of T cells and NK cells to ESCs, iMSCs, and their more terminally differentiated derivatives such as osteoblasts can be examined.

In certain particular embodiments, the mesenchymal stem cells are capable of differentiating into non-limiting examples of cell types such as marrow stromal cells, adipocytes, osteoblasts, osteocytes and chondrocytes. The cells of the invention are differentiated into mesenchymal stem cells either before or after the cells are administered to a patient. In certain preferred embodiments, the cell is a human cell and the patient is a human. In vitro mesenchymal differentiation can be performed according to established protocols. See for example, Deyle et al., supra.

In yet other particular embodiments, the cells of the invention can be differentiated into insulin-producing pancreatic islet cells. In certain embodiments, the cells of the invention express one or more HLA class II proteins or single chain fusion HLA class II proteins and optionally also one or more single chain fusion HLA class I proteins. The cells of the invention can be used to treat insulin-dependent diabetes mellitus. Advantageously, the transplanted cells do not need to reconstitute a functioning pancreas: they just need to secrete insulin in response to glucose levels. Therefore the treatment can succeed with different cell doses, with cells that are not perfectly differentiated into adult cell types, and when cells are transplanted into an ectopic location. Specific auto-antigens such as those derived from GAD65 or Insulin can cause autoimmune destruction of 0 cells in diabetes (Di Lorenzo et al., 2007, Clin Exp Immunol 148, 1-16). Thus, HLA class II deficient, HLA class I deficient, or HLA class I/class II deficient cells expressing single chain fusion HLA protein presenting a predetermined peptide antigen provide additional advantages in that they do not present these auto-antigens and can avoid autoimmune rejection and prevent a relapse of diabetes after transplantation.

The cells of the invention can be differentiated into pancreatic cells as described previously, which employs exposure of cells to different cytokines and drugs to promote sequential formation of mesendoderm, definitive endoderm, and pancreatic progenitors (Kroon et al., 2008, Nat Biotechnol 26, 443-452). These cells can be further cultured in implants in immunodeficient mice. The wild-type cells and cells of the invention with or without expressing one or more single chain fusion HLA class II proteins or both one or more single chain fusion HLA class II proteins and optionally also one or more single chain HLA class I proteins can be analyzed at different developmental stages for their reactivity with T cells and NK cells.

The cells of the invention are differentiated into pancreatic islet cell either before or after patient administration. In certain preferred embodiments, the cell is a human cell and the patient is a human. In vitro hematopoietic differentiation can be performed according to established protocols. See for example, Kroon et al., 2008, Nat Biotechnol 26, 443-452.

In certain other particular embodiments, the cells of the invention can be differentiated into cardiomyocytes. In certain embodiments, the cells of the invention further express one or more HLA class II proteins or single chain fusion HLA class II proteins and optionally also one or more single chain fusion HLA class I proteins. The common clinical problems of myocardial infarction and congestive heart failure can be treated by transplanting healthy stem cell-derived cardiomyocytes that engraft and re-establish functional myocardium. The cells of the invention-derived cardiomyocytes allow these treatments to proceed with pre-packaged cells and avoid the immunosuppression currently required for allogeneic heart transplants. Physiologically relevant tests can be performed on the cardiomyocytes derived from the cells of the invention, such as electrical conduction and contraction studies. HLA class II deficient, HLA class I deficient, or HLA class I/class II deficient stem cells or differentiated cardiomyocytes with or without expressing a single chain fusion HLA class I protein can be tested to determine their immunological reactivity when expressing cardiomyocyte genes, and to establish which HLA modifications minimize these immune responses.

The cells of the invention can be differentiated into cardiomyocytes either before or after the cells are administered to a patient. In certain preferred embodiments, the cell is a human cell and the patient is a human. In certain embodiments, the cells of the invention are differentiated into cardiomyocytes for treating diseases including without limitation myocardial infarction and congestive heart failure. In vitro cardiomyocyte differentiation can be performed according to established protocols. See for example, Laflamme et al., 2007, Nat Biotechnol 25, 1015-1024.

In yet other particular embodiments, the cells of the invention can be differentiated into keratinocytes. In certain embodiments, the cells of the invention used for differentiation into keratinocytes express one or more single chain fusion HLA class II proteins and optionally also one or more HLA class II proteins or single chain fusion HLA class I proteins. Severe burns and genetic skin conditions require treatment with skin grafts, and this is currently done with a variety of cell sources such as porcine skin grafts and cultured autologous human keratinocytes. Keratinocytes derived from the cells of the invention can provide a major clinical advance, since burns could be treated as an emergency with pre-packaged cells, and genetic diseases such as epidermolysis bullosum can be treated with otherwise normal HLA class II deficient, HLA class I deficient, or HLA class I/class II deficient cells that do not require correction of the responsible genetic mutations. In many cases the cells only need to engraft long enough for neighboring host cells to repopulate the affected area. For in vivo differentiation, the cells of the invention can be embedded in polyvinyl alcohol sponge (PVA)-collagen gel implants for transplantation into a recipient. The cells of the invention can be differentiated into keratinocytes either before or after transplantation. In certain preferred embodiments, the cell is a human cell and the patient is a human.

In yet another aspect, the invention provides a use of the cells of the invention for the preparation for a medicament for transplantation. In a related aspect, the invention provides a use of the cells of the invention for the preparation for a medicament for treating a disease condition.

Further, the cells of the invention can serve as a research tool to provide a system for studying the functions of immunoregulatory proteins in a HLA class II deficient, HLA class I deficient, or HLA class I/class II deficient genetic background. In certain embodiments, the cells of the invention further express one or more HLA class II proteins or single chain fusion HLA class II proteins and optionally also one or more single chain fusion HLA class I proteins. Accordingly, in a related aspect, the invention provides a method of determining the function of an immunoregulatory protein comprising the steps of introducing one or more immunoregulatory genes into the cells of the invention of the invention and assaying for the activities of the immunoregulatory genes. In certain preferred embodiments, the cell is a human cell. For example, the cells of the invention can be used to study the function of an immune regulatory gene, or to study an immune response, in the absence of unwanted HLA class II antigens or HLA class I/class II antigens. In a further related aspect, the invention provides a method of identifying a compound or molecule that modulates the function of the immunoregulatory protein comprising the steps of contacting the HLA class II deficient cells, HLA class I deficient cells, or HLA class I/class II deficient cells comprising the one or more immunoregulatory genes with a compound or molecule of interest and assaying for the activities of the immunoregulatory genes. In certain preferred embodiments, the cell is a human cell.

In a related aspect, the invention provides kits that comprise the HLA class II deficient, HLA class I deficient, or HLA class I/class II deficient cells of the invention and an implant, wherein the implant comprises the cells of the invention.

In yet another aspect, the invention provides an in vivo research tool in a mammal, particular in a non-human primate, that are administered the cells of the invention, for studying the functions of immunoregulatory genes, or identifying a compound that modulates the function of an immunoregulatory gene in the administered cells in an HLA class II deficient, HLA class I deficient, or HLA class I/class II deficient genetic background. In certain embodiments, the cells of the invention further express one or more HLA class II proteins or single chain fusion HLA class II proteins and optionally also one or more single chain fusion HLA class I proteins.

Mice, especially immune deficient mice, have been used as a model system for studying human cells in vivo. Human stem cells can behave differently in mice. In addition, the mouse and human immune systems have different HLA class II genes, NK cell receptors and non-classical MHC class I genes (e.g. HLA-E, F and G). Therefore, a Macaca nemestrina (Mn, pigtailed macaque) model can be developed to study the cells of the invention. The Macaca mulatta genome has been sequenced, which is highly homologous to the nemestrina genome. Further, the organization of macaque MHC loci is similar to human HLA, including the non-classical genes. Homologs of the human HLA-E and HLA-G genes have been identified in macaques. The macaque MHC loci also contain homologs of many human NK cell receptors. Human and Mn HLA class II deficient, HLA class I deficient, or HLA class I/class II deficient ESCs can be used for transplantation in macaques.

MHC class II-deficient of MHC class I/class II-deficient macaque ESCs can be developed using the same AAV-mediated gene targeting strategy described for human cells. Mn versions of the single chain fusion HLA class II proteins and optionally single chain fusion HLA class I proteins are expressed in the HLA class II or HLA class II/class I deficient macaques ESCs using the analogous viral vectors as described above.

Cells can be expanded in vitro and labeled with a vector expressing GFP for subsequent identification of transplanted cells. The cells can be embedded in polyvinyl alcohol sponge (PVA)-collagen gel implants, and placed subdermally into macaques. The implants can be harvested, sectioned and stained to determine the cell types that are present. Specific antibodies can be used to identify the differentiated cell types formed by the transplanted cells.

Any and every embodiment described above applies to any and every aspect of the invention, unless the context clearly indicates otherwise. All embodiments within and between different aspects can be combined unless the context clearly dictates otherwise.

### EXAMPLES

### Example 1 Construction of Human Embryonic Stem Cells with Knockout Mutation in RFXANK Gene

FIG. 1 shows the structure of exemplary two adeno-associated virus (AAV) gene targeting vectors, designed to insert either a TKNeo (AAV-RFXANK-ETKNpA) or HyTK (AAV-RFXANK-HyTK) gene controlled by an EF1alpha promoter (EF) into exon 3 of the RFXANK gene, which is also shown below the vectors. Selection of vector-infected cells with G418 or hygromycin (Hygro) allows one to isolate cells targeted by the TKNeo or HyTK vectors respectively. Subsequent expression of Cre recombinase and selection with gancyclovir (GCV) then allows one to isolate clones that have removed the TKNeo or HyTK genes, leaving behind two inactivated RFXANK alleles with stop codons in all 3 reading frames, a loxP site, and a polyadenylation site (StopX3-loxP-pA). LoxP is the recombination site for Cre recombinase. ITR is a vector inverted terminal repeat. Similar vectors could be designed to target other genes.

The AAV-RFXANK-ETKNpA vector (SEQ ID NO: 56) was used to create a knockout mutation in a first allele of the RFXANK gene. Human embryonic stem cells were infected with AAV-RFXANK-ETKNpA and screened for targeting by PCR using a forward primer homologous to the neomycin sequence of the selection cassette and a reverse primer homologous to the RFXANK gene which was outside the targeting homology arm, as indicated by the arrows above. As shown in FIG. 2, 5 positive clones of the correct size are shown above out of 40 clones screened yielding a targeting frequency of 12.5%.

What is claimed is:

1. A method of transplantation in a patient in need thereof, comprising administering to the patient an effective amount of a cell comprising a genetically engineered disruption of all copies of a human leukocyte antigen (HLA) class II-related gene, wherein the cell is a human cell, wherein:
a. the HLA class II-related gene is selected from the group consisting of regulatory factor X-associated ankyrin-containing protein, regulatory factor 5, regulatory factor X associated protein, class II transactivator;
b. the cell further comprises one or more polynucleotide, wherein the one or more polynucleotide is capable of encoding a single chain fusion HLA class I protein, wherein the single chain fusion HLA class I protein comprises at least a portion of the β-microglobulin (B2M) gene (SEQ ID NO: 2) covalently linked to at least a portion of a non-classical HLA class Iα chain;
c. the cell further comprises a genetically engineered disruption in the endogenous B2M gene (SEQ ID NO: 1); and
d. wherein the single chain fusion HLA class I protein is capable of engaging inhibitory receptors on the surface of natural killer cells.

2. The method of claim 1, wherein the HLA class II-related gene is regulatory factor X associated ankyrin-containing protein.

3. The method of claim 1, wherein the single chain fusion HLA class I protein comprises at least a portion of B2M (SEQ ID NO: 2) covalently linked to at least a portion of HLA-E (SEQ ID NO: 10), HLA-F (SEQ ID NO: 12) or HLA-G (SEQ ID NO: 14).

4. The method of claim 1, wherein the single chain fusion HLA class I protein comprises at least a portion of B2M (SEQ ID NO: 2) covalently linked to at least a portion of HLA-E (SEQ ID NO: 10).

5. The method of claim 1, wherein the single chain fusion HLA class I protein comprises at least a portion of B2M (SEQ ID NO: 2) covalently linked to at least a portion of HLA-F (SEQ ID NO: 12).

6. The method of claim 1, wherein the single chain fusion HLA class I protein comprises at least a portion of B2M (SEQ ID NO: 2) covalently linked to at least a portion of HLA-G (SEQ ID NO: 14).

7. The method of claim 1, wherein the cell comprises genetically engineered disruptions in all copies of the endogenous cell B2M gene.

8. The method of claim 1, wherein the cell has a normal karyotype.

9. The method of claim 1, wherein the cell is a non-transformed cell.

10. The method of claim 1, wherein the cell is a stem cell.

11. The method of claim 10, wherein the stem cell is selected from the group consisting of a pluripotent stem cell, a hematopoietic stem cell, an embryonic stem cell, an induced pluripotent stem cell, an adult stem cell, a liver stem cell, a neural stem cell, a pancreatic stem cell and a mesenchymal stem cell.

12. The method of claim 10, wherein the stem cell is a pluripotent stem cell.

13. The method of claim 1, wherein the cell is a differentiated cell.

14. The method of claim 13, wherein the differentiated cell is selected from the group consisting of a dendritic cell, a pancreatic islet cell, a liver cell, a muscle cell, a keratinocyte, a neuronal cell, a hematopoietic cell, a lymphocyte, a red blood cell, a platelet, a skeletal muscle cell, an ocular cell, a mesenchymal cell, a fibroblast, a lung cell, a GI tract cell, a vascular cell, an endocrine cell, an adipocyte, a marrow stromal cell, an osteoblast, a chrondrocyte, and a cardiomyocyte.

15. The method of claim 1, wherein the cell is an allogeneic cell.

16. The method of claim 1, wherein the patient is immunocompetent.

17. The method of claim 1, wherein the patient is not concurrently taking immune suppressant therapies.

18. The method of claim 1, wherein the patient has a disease or injury to a tissue or organs.

19. The method of claim 1, wherein the patient has one or more genetic defects or mutation in a tissue or organ.

20. The method of claim 18, wherein the tissue or organ is selected from the group consisting of: liver, kidney, pancreas, lung, brain, muscle, heart, gastrointestinal tract, nervous system, skin, bone marrow, fat, connective tissue, immune system, and blood vessels.

21. The method of claim 19, wherein the tissue or organ is selected from the group consisting of: liver, kidney, pancreas, lung, brain, muscle, heart, gastrointestinal tract, nervous system, skin, bone marrow, fat, connective tissue, immune system, and blood vessels.

22. A method of treating a disease condition in a patient in need thereof, comprising administering to the patient an effective amount of a cell comprising a genetically engineered disruption of all copies of a human leukocyte antigen (HLA) class II-related gene, wherein the cell is a human cell, wherein:
a. the HLA class II-related gene is selected from
b. the cell further comprises one or more polynucleotide, wherein the one or more polynucleotide is capable of encoding a single chain fusion HLA class I protein, wherein the single chain fusion HLA class I protein comprises at least a portion of the β-microglobulin (B2M) gene (SEQ ID NO: 2) covalently linked to at least a portion of a non-classical HLA class Iα chain;
c. the cell further comprises a genetically engineered disruption in the endogenous B2M gene (SEQ ID NO: 1); and
d. wherein the single chain fusion HLA class I protein is capable of engaging inhibitory receptors on the surface of natural killer cells.

23. The method of claim 22, wherein the disease is selected from the group consisting of: diabetes, autoimmune disease, cancer, infection, anemia, cytopenia, myocardial infarction, heart failure, skeletal or joint condition, osteogenesis imperfecta or burns.


SEQUENCE LISTING (56 sequences)

SEQ ID NO: 1 - DNA, 987 bp, Homo sapiens
  source 1..987: mol_type = genomic DNA
  CDS 61..420
aatataagtggaggcgtcgcgctggcgggcattcctgaagctgacagcattcgggccgagatgtctcgctccgtggccttagctgtgctcgcgctactctctctttctggcctggaggctatccagcgtactccaaagattcaggtttactcacgtcatccagcagagaatggaaagtcaaatttcctgaattgctatgtgtctgggtttcatccatccgacattgaagttgacttactgaagaatggagagagaattgaaaaagtggagcattcagacttgtctttcagcaaggactggtctttctatctcttgtactacactgaattcacccccactgaaaaagatgagtatgcctgccgtgtgaaccatgtgactttgtcacagcccaagatagttaagtgggatcgagacatgtaagcagcatcatggaggtttgaagatgccgcatttggattggatgaattccaaattctgcttgcttgctttttaatattgatatgcttatacacttacactttatgcacaaaatgtagggttataataatgttaacatggacatgatcttctttataattctactttgagtgctgtctccatgtttgatgtatctgagcaggttgctccacaggtagctctaggagggctggcaacttagaggtggggagcagagaattctcttatccaacatcaacatcttggtcagatttgaactcttcaatctcttgcactcaaagcttgttaagatagttaagcgtgcataagttaacttccaatttacatactctgcttagaatttgggggaaaatttagaaatataattgacaggattattggaaatttgttataatgaatgaaacattttgtcatataagattcatatttacttcttatacatttgataaagtaaggcatggttgtggttaatctggtttatttttgttccacaagttaaataaatcataaaacttgatgtgttatctctta

SEQ ID NO: 2 - protein, 119 aa, Homo sapiens
MSRSVALAVLALLSLSGLEAIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDM

SEQ ID NO: 3 - DNA, 1611 bp, Homo sapiens
  source 1..1611: mol_type = genomic DNA
  CDS 85..1182
gagaagccaatcagtgtcgtcgcggtcgctgttctaaagtccgcacgcacccaccgggactcagattctccccagacgccgaggatggccgtcatggcgccccgaaccctcctcctgctactctcgggggccctggccctgacccagacctgggcgggctcccactccatgaggtatttcttcacatccgtgtcccggcccggccgcggggagccccgcttcatcgccgtgggctacgtggacgacacgcagttcgtgcggttcgacagcgacgccgcgagccagaagatggagccgcgggcgccgtggatagagcaggaggggccggagtattgggaccaggagacacggaatatgaaggcccactcacagactgaccgagcgaacctggggaccctgcgcggctactacaaccagagcgaggacggttctcacaccatccagataatgtatggctgcgacgtggggccggacgggcgcttcctccgcgggtaccggcaggacgcctacgacggcaaggattacatcgccctgaacgaggacctgcgctcttggaccgcggcggacatggcagctcagatcaccaagcgcaagtgggaggcggtccatgcggcggagcagcggagagtctacctggagggccggtgcgtggacgggctccgcagatacctggagaacgggaaggagacgctgcagcgcacggacccccccaagacacatatgacccaccaccccatctctgaccatgaggccaccctgaggtgctgggccctgggcttctaccctgcggagatcacactgacctggcagcgggatggggaggaccagacccaggacacggagctcgtggagaccaggcctgcaggggatggaaccttccagaagtgggcggctgtggtggtgccttctggagaggagcagagatacacctgccatgtgcagcatgagggtctgcccaagcccctcaccctgagatgggagctgtcttcccagcccaccatccccatcgtgggcatcattgctggcctggttctccttggagctgtgatcactggagctgtggtcgctgccgtgatgtggaggaggaagagctcagatagaaaaggagggagttacactcaggctgcaagcagtgacagtgcccagggctctgatgtgtctctcacagcttgtaaagtgtgagacagctgccttgtgtgggactgagaggcaagagttgttcctgcccttccctttgtgacttgaagaaccctgactttgtttctgcaaaggcacctgcatgtgtctgtgttcgtgtaggcataatgtgaggaggtggggagagcaccccacccccatgtccaccatgaccctcttcccacgctgacctgtgctccctctccaatcatctttcctgttccagagaggtggggctgaggtgtctccatctctgtctcaacttcatggtgcactgagctgtaacttcttccttccctattaaaattagaacctgagtataaatttactttctcaaattcttgccatgagaggttgatgagttaattaaaggagaagattcctaaaatttgagagacaaaattaatggaacgcatgagaaccttccagagtcca

SEQ ID NO: 4 - protein, 365 aa, Homo sapiens
MAVMAPRTLLLLLSGALALTQTWAGSHSMRYFFTSVSRPGRGEPRFIAVGYVDDTQFVRFDSDAASQKMEPRAPWIEQEGPEYWDQETRNMKAHSQTDRANLGTLRGYYNQSEDGSHTIQIMYGCDVGPDGRFLRGYRQDAYDGKDYIALNEDLRSWTAADMAAQITKRKWEAVHAAEQRRVYLEGRCVDGLRRYLENGKETLQRTDPPKTHMTHHPISDHEATLRCWALGFYPAEITLTWQRDGEDQTQDTELVETRPAGDGTFQKWAAVVVPSGEEQRYTCHVQHEGLPKPLTLRWELSSQPTIPIVGIIAGLVLLGAVITGAVVAAVMWRRKSSDRKGGSYTQAASSDSAQGSDVSLTACKV

SEQ ID NO: 5 - DNA, 1578 bp, Homo sapiens
  source 1..1578: mol_type = genomic DNA
  CDS 55..1143
agttctaaagtccccacgcacccacccggactcagagtctcctcagacgccgagatgctggtcatggcgccccgaaccgtcctcctgctgctctcggcggccctggccctgaccgagacctgggccggctcccactccatgaggtatttctacacctccgtgtcccggcccggccgcggggagccccgcttcatctcagtgggctacgtggacgacacccagttcgtgaggttcgacagcgacgccgcgagtccgagagaggagccgcgggcgccgtggatagagcaggaggggccggagtattgggaccggaacacacagatctacaaggcccaggcacagactgaccgagagagcctgcggaacctgcgcggctactacaaccagagcgaggccgggtctcacaccctccagagcatgtacggctgcgacgtggggccggacgggcgcctcctccgcgggcatgaccagtacgcctacgacggcaaggattacatcgccctgaacgaggacctgcgctcctggaccgccgcggacacggcggctcagatcacccagcgcaagtgggaggcggcccgtgaggcggagcagcggagagcctacctggagggcgagtgcgtggagtggctccgcagatacctggagaacgggaaggacaagctggagcgcgctgaccccccaaagacacacgtgacccaccaccccatctctgaccatgaggccaccctgaggtgctgggccctgggtttctaccctgcggagatcacactgacctggcagcgggatggcgaggaccaaactcaggacactgagcttgtggagaccagaccagcaggagatagaaccttccagaagtgggcagctgtggtggtgccttctggagaagagcagagatacacatgccatgtacagcatgaggggctgccgaagcccctcaccctgagatgggagccgtcttcccagtccaccgtccccatcgtgggcattgttgctggcctggctgtcctagcagttgtggtcatcggagctgtggtcgctgctgtgatgtgtaggaggaagagttcaggtggaaaaggagggagctactctcaggctgcgtgcagcgacagtgcccagggctctgatgtgtctctcacagcttgaaaagcctgagacagctgtcttgtgagggactgagatgcaggatttcttcacgcctcccctttgtgacttcaagagcctctggcatctctttctgcaaaggcacctgaatgtgtctgcgtccctgttagcataatgtgaggaggtggagagacagcccacccttgtgtccactgtgacccctgttcccatgctgacctgtgtttcctccccagtcatctttcttgttccagagaggtggggctggatgtctccatctctgtctcaactttacgtgcactgagctgcaacttcttacttccctactgaaaataagaatctgaatataaatttgttttctcaaatatttgctatgagaggttgatggattaattaaataagtcaattcctggaatttgagagagcaaataaagacctgagaaccttccagaaaaaaaa

SEQ ID NO: 6 - protein, 362 aa, Homo sapiens
MLVMAPRTVLLLLSAALALTETWAGSHSMRYFYTSVSRPGRGEPRFISVGYVDDTQFVRFDSDAASPREEPRAPWIEQEGPEYWDRNTQIYKAQAQTDRESLRNLRGYYNQSEAGSHTLQSMYGCDVGPDGRLLRGHDQYAYDGKDYIALNEDLRSWTAADTAAQITQRKWEAAREAEQRRAYLEGECVEWLRRYLENGKDKLERADPPKTHVTHHPISDHEATLRCWALGFYPAEITLTWQRDGEDQTQDTELVETRPAGDRTFQKWAAVVVPSGEEQRYTCHVQHEGLPKPLTLRWEPSSQSTVPIVGIVAGLAVLAVVVIGAVVAAVMCRRKSSGGKGGSYSQAACSDSAQGSDVSLTA

SEQ ID NO: 7 - DNA, 1586 bp, Homo sapiens
  source 1..1586: mol_type = genomic DNA
  CDS 66..1166
tccgcagtcccggttctaaagtccccagtcacccacccggactcacattctccccagaggccgagatgcgggtcatggcgccccgagccctcctcctgctgctctcgggaggcctggccctgaccgagacctgggcctgctcccactccatgaggtatttcgacaccgccgtgtcccggcccggccgcggagagccccgcttcatctcagtgggctacgtggacgacacgcagttcgtgcggttcgacagcgacgccgcgagtccgagaggggagccgcgggcgccgtgggtggagcaggaggggccggagtattgggaccgggagacacagaagtacaagcgccaggcacaggctgaccgagtgagcctgcggaacctgcgcggctactacaaccagagcgaggacgggtctcacaccctccagaggatgtctggctgcgacctggggcccgacgggcgcctcctccgcgggtatgaccagtccgcctacgacggcaaggattacatcgccctgaacgaggacctgcgctcctggaccgccgcggacaccgcggctcagatcacccagcgcaagttggaggcggcccgtgcggcggagcagctgagagcctacctggagggcacgtgcgtggagtggctccgcagatacctggagaacgggaaggagacgctgcagcgcgcagaacccccaaagacacacgtgacccaccaccccctctctgaccatgaggccaccctgaggtgctgggccctgggcttctaccctgcggagatcacactgacctggcagcgggatggggaggaccagacccaggacaccgagcttgtggagaccaggccagcaggagatggaaccttccagaagtgggcagctgtggtggtgccttctggacaagagcagagatacacgtgccatatgcagcacgaggggctgcaagagcccctcaccctgagctgggagccatcttcccagcccaccatccccatcatgggcatcgttgctggcctggctgtcctggttgtcctagctgtccttggagctgtggtcaccgctatgatgtgtaggaggaagagctcaggtggaaaaggagggagctgctctcaggctgcgtgcagcaacagtgcccagggctctgatgagtctctcatcacttgtaaagcctgagacagctgcctgtgtgggactgagatgcaggatttcttcacacctctcctttgtgacttcaagagcctctggcatctctttctgcaaaggcacctgaatgtgtctgcgttcctgttagcataatgtgaggaggtggagagacagcccacccccgtgtccaccgtgacccctgtccccacactgacctgtgttccctccccgatcatctttcctgttccagagaggtggggctggatgtctccatctctgtctcaaattcatggtgcactgagctgcaacttcttacttccctaatgaagttaagaacctgaatataaatttgtgttctcaaatatttgctatgaagcgttgatggattaattaaataagtcaattcctagaagttgagagagcaaataaagacctgagaaccttccagaa

SEQ ID NO: 8 - protein, 366 aa, Homo sapiens
MRVMAPRALLLLLSGGLALTETWACSHSMRYFDTAVSRPGRGEPRFISVGYVDDTQFVRFDSDAASPRGEPRAPWVEQEGPEYWDRETQKYKRQAQADRVSLRNLRGYYNQSEDGSHTLQRMSGCDLGPDGRLLRGYDQSAYDGKDYIALNEDLRSWTAADTAAQITQRKLEAARAAEQLRAYLEGTCVEWLRRYLENGKETLQRAEPPKTHVTHHPLSDHEATLRCWALGFYPAEITLTWQRDGEDQTQDTELVETRPAGDGTFQKWAAVVVPSGQEQRYTCHMQHEGLQEPLTLSWEPSSQPTIPIMGIVAGLAVLVVLAVLGAVVTAMMCRRKSSGGKGGSCSQAACSNSAQGSDESLITCKA

SEQ ID NO: 9 - DNA, 2679 bp, Homo sapiens
  source 1..2679: mol_type = genomic DNA
  CDS 127..1203
gcagactcagttctcattcccaatgggtgtcgggtttctagagaagccaatcagcgtcgccacgactcccgactataaagtccccatccggactcaagaagttctcaggactcagaggctgggatcatggtagatggaaccctccttttactcctctcggaggccctggcccttacccagacctgggcgggctcccactccttgaagtatttccacacttccgtgtcccggcccggccgcggggagccccgcttcatctctgtgggctacgtggacgacacccagttcgtgcgcttcgacaacgacgccgcgagtccgaggatggtgccgcgggcgccgtggatggagcaggaggggtcagagtattgggaccgggagacacggagcgccagggacaccgcacagattttccgagtgaatctgcggacgctgcgcggctactacaatcagagcgaggccgggtctcacaccctgcagtggatgcatggctgcgagctggggcccgacgggcgcttcctccgcgggtatgaacagttcgcctacgacggcaaggattatctcaccctgaatgaggacctgcgctcctggaccgcggtggacacggcggctcagatctccgagcaaaagtcaaatgatgcctctgaggcggagcaccagagagcctacctggaagacacatgcgtggagtggctccacaaatacctggagaaggggaaggagacgctgcttcacctggagcccccaaagacacacgtgactcaccaccccatctctgaccatgaggccaccctgaggtgctgggccctgggcttctaccctgcggagatcacactgacctggcagcaggatggggagggccatacccaggacacggagctcgtggagaccaggcctgcaggggatggaaccttccagaagtgggcagctgtggtggtgccttctggagaggagcagagatacacgtgccatgtgcagcatgaggggctacccgagcccgtcaccctgagatggaagccggcttcccagcccaccatccccatcgtgggcatcattgctggcctggttctccttggatctgtggtctctggagctgtggttgctgctgtgatatggaggaagaagagctcaggtggaaaaggagggagctactctaaggctgagtggagcgacagtgcccaggggtctgagtctcacagcttgtaaagcctgagacagctgccttgtgtgcgactgagatgcacagctgccttgtgtgcgactgagatgcaggatttcctcacgcctcccctatgtgtcttaggggactctggcttctctttttgcaagggcctctgaatctgtctgtgtccctgttagcacaatgtgaggaggtagagaaacagtccacctctgtgtctaccatgacccccttcctcacactgacctgtgttccttccctgttctcttttctattaaaaataagaacctgggcagagtgcggcagctcatgcctgtaatcccagcacttagggaggccgaggagggcagatcacgaggtcaggagatcgaaaccatcctggctaacacggtgaaaccccgtctctactaaaaaatacaaaaaattagctgggcgcagaggcacgggcctgtagtcccagctactcaggaggcggaggcaggagaatggcgtcaacccgggaggcggaggttgcagtgagccaggattgtgcgactgcactccagcctgggtgacagggtgaaacgccatctcaaaaaataaaaattgaaaaataaaaaaagaacctggatctcaatttaatttttcatattcttgcaatgaaatggacttgaggaagctaagatcatagctagaaatacagataattccacagcacatctctagcaaatttagcctattcctattctctagcctattccttaccacctgtaatcttgaccatataccttggagttgaatattgttttcatactgctgtggtttgaatgttccctccaacactcatgttgagacttaatccctaatgtggcaatactgaaaggtggggcctttgagatgtgattggatcgtaaggctgtgccttcattcatgggttaatggattaatgggttatcacaggaatgggactggtggctttataagaagaggaaaagagaactgagctagcatgcccagcccacagagagcctccactagagtgatgctaagtggaaatgtgaggtgcagctgccacagagggcccccaccagggaaatgtctagtgtctagtggatccaggccacaggagagagtgccttgtggagcgctgggagcaggacctgaccaccaccaggaccccagaactgtggagtcagtggcagcatgcagcgcccccttgggaaagctttaggcaccagcctgcaacccattcgagcagccacgtaggctgcacccagcaaagccacaggcacggggctacctgaggccttgggggcccaatccctgctccagtgtgtccgtgaggcagcacacgaagtcaaaagagattattctcttcccacagataccttttctctcccatgaccctttaacagcatctgcttcattcccctcaccttcccaggctgatctgaggtaaactttgaagtaaaataaaagctgtgtttgagcatcatttgtatttcaaaaaaaaaaaaaaaaaa

SEQ ID NO: 10 - protein, 358 aa, Homo sapiens
MVDGTLLLLLSEALALTQTWAGSHSLKYFHTSVSRPGRGEPRFISVGYVDDTQFVRFDNDAASPRMVPRAPWMEQEGSEYWDRETRSARDTAQIFRVNLRTLRGYYNQSEAGSHTLQWMHGCELGPDGRFLRGYEQFAYDGKDYLTLNEDLRSWTAVDTAAQISEQKSNDASEAEHQRAYLEDTCVEWLHKYLEKGKETLLHLEPPKTHVTHHPISDHEATLRCWALGFYPAEITLTWQQDGEGHTQDTELVETRPAGDGTFQKWAAVVVPSGEEQRYTCHVQHEGLPEPVTLRWKPASQPTIPIVGIIAGLVLLGSVVSGAVVAAVIWRKKSSGGKGGSYSKAEWSDSAQGSESHSL

SEQ ID NO: 11 - DNA, 1301 bp, Homo sapiens
  source 1..1301: mol_type = genomic DNA
  CDS 125..1165
tttctcactcccattgggcgtcgcgtttctagagaagccaatcagtgtcgccgcagttcccaggttctaaagtcccacgcaccccgcgggactcatatttttcccagacgcggaggttggggtcatggcgccccgaagcctcctcctgctgctctcaggggccctggccctgaccgatacttgggcgggctcccactccttgaggtatttcagcaccgctgtgtcgcggcccggccgcggggagccccgctacatcgccgtggagtacgtagacgacacgcaattcctgcggttcgacagcgacgccgcgattccgaggatggagccgcgggagccgtgggtggagcaagaggggccgcagtattgggagtggaccacagggtacgccaaggccaacgcacagactgaccgagtggccctgaggaacctgctccgccgctacaaccagagcgaggctgggtctcacaccctccagggaatgaatggctgcgacatggggcccgacggacgcctcctccgcgggtatcaccagcacgcgtacgacggcaaggattacatctccctgaacgaggacctgcgctcctggaccgcggcggacaccgtggctcagatcacccagcgcttctatgaggcagaggaatatgcagaggagttcaggacctacctggagggcgagtgcctggagttgctccgcagatacttggagaatgggaaggagacgctacagcgcgcagatcctccaaaggcacacgttgcccaccaccccatctctgaccatgaggccaccctgaggtgctgggccctgggcttctaccctgcggagatcacgctgacctggcagcgggatggggaggaacagacccaggacacagagcttgtggagaccaggcctgcaggggatggaaccttccagaagtgggccgctgtggtggtgcctcctggagaggaacagagatacacatgccatgtgcagcacgaggggctgccccagcccctcatcctgagatgggagcagtctccccagcccaccatccccatcgtgggcatcgttgctggccttgttgtccttggagctgtggtcactggagctgtggtcgctgctgtgatgtggaggaagaagagctcagatagaaacagagggagctactctcaggctgcagtgtgagacagcttccttgtgtgggactgagaagcaagatatcaatgtagcagaattgcacttgtgcctcacgaacatacataaattttaaaaataaagaataaaaatatatctttttatagataaaaaaaaaaaaaaaaaa

SEQ ID NO: 12 - protein, 346 aa, Homo sapiens
MAPRSLLLLLSGALALTDTWAGSHSLRYFSTAVSRPGRGEPRYIAVEYVDDTQFLRFDSDAAIPRMEPREPWVEQEGPQYWEWTTGYAKANAQTDRVALRNLLRRYNQSEAGSHTLQGMNGCDMGPDGRLLRGYHQHAYDGKDYISLNEDLRSWTAADTVAQITQRFYEAEEYAEEFRTYLEGECLELLRRYLENGKETLQRADPPKAHVAHHPISDHEATLRCWALGFYPAEITLTWQRDGEEQTQDTELVETRPAGDGTFQKWAAVVVPPGEEQRYTCHVQHEGLPQPLILRWEQSPQPTIPIVGIVAGLVVLGAVVTGAVVAAVMWRKKSSDRNRGSYSQAAV

SEQ ID NO: 13 - DNA, 1578 bp, Homo sapiens
  source 1..1578: mol_type = genomic DNA
  CDS 179..1195
agtgtggtactttgtcttgaggagatgtcctggactcacacggaaacttagggctacggaatgaagttctcactcccattaggtgacaggtttttagagaagccaatcagcgtcgccgcggtcctggttctaaagtcctcgctcacccacccggactcattctccccagacgccaaggatggtggtcatggcgccccgaaccctcttcctgctgctctcgggggccctgaccctgaccgagacctgggcgggctcccactccatgaggtatttcagcgccgccgtgtcccggcccggccgcggggagccccgcttcatcgccatgggctacgtggacgacacgcagttcgtgcggttcgacagcgactcggcgtgtccgaggatggagccgcgggcgccgtgggtggagcaggaggggccggagtattgggaagaggagacacggaacaccaaggcccacgcacagactgacagaatgaacctgcagaccctgcgcggctactacaaccagagcgaggccagttctcacaccctccagtggatgattggctgcgacctggggtccgacggacgcctcctccgcgggtatgaacagtatgcctacgatggcaaggattacctcgccctgaacgaggacctgcgctcctggaccgcagcggacactgcggctcagatctccaagcgcaagtgtgaggcggccaatgtggctgaacaaaggagagcctacctggagggcacgtgcgtggagtggctccacagatacctggagaacgggaaggagatgctgcagcgcgcggacccccccaagacacacgtgacccaccaccctgtctttgactatgaggccaccctgaggtgctgggccctgggcttctaccctgcggagatcatactgacctggcagcgggatggggaggaccagacccaggacgtggagctcgtggagaccaggcctgcaggggatggaaccttccagaagtgggcagctgtggtggtgccttctggagaggagcagagatacacgtgccatgtgcagcatgaggggctgccggagcccctcatgctgagatggaagcagtcttccctgcccaccatccccatcatgggtatcgttgctggcctggttgtccttgcagctgtagtcactggagctgcggtcgctgctgtgctgtggagaaagaagagctcagattgaaaaggagggagctactctcaggctgcaatgtgaaacagctgccctgtgtgggactgagtggcaagtccctttgtgacttcaagaaccctgactcctctttgtgcagagaccagcccacccctgtgcccaccatgaccctcttcctcatgctgaactgcattccttccccaatcacctttcctgttccagaaaaggggctgggatgtctccgtctctgtctcaaatttgtggtccactgagctataacttacttctgtattaaaattagaatctgagtataaatttactttttcaaattatttccaagagagattgatgggttaattaaaggagaagattcctgaaatttgagagacaaaataaatggaagacatgagaacttt

SEQ ID NO: 14 - protein, 338 aa, Homo sapiens
MVVMAPRTLFLLLSGALTLTETWAGSHSMRYFSAAVSRPGRGEPRFIAMGYVDDTQFVRFDSDSACPRMEPRAPWVEQEGPEYWEEETRNTKAHAQTDRMNLQTLRGYYNQSEASSHTLQWMIGCDLGSDGRLLRGYEQYAYDGKDYLALNEDLRSWTAADTAAQISKRKCEAANVAEQRRAYLEGTCVEWLHRYLENGKEMLQRADPPKTHVTHHPVFDYEATLRCWALGFYPAEIILTWQRDGEDQTQDVELVETRPAGDGTFQKWAAVVVPSGEEQRYTCHVQHEGLPEPLMLRWKQSSLPTIPIMGIVAGLVVLAAVVTGAAVAAVLWRKKSSD

SEQ ID NO: 15 - DNA, 9232 bp, synthetic construct
  misc_feature 1..9232: note = Vector sequence for human B2M-HLA-A0201 fusion protein
  source 1..9232: mol_type = other DNA
ctcgaggagcttggcccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacgctaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagcttctagattgtacgggagctcttcactactcgctgcgtcgagagtgtacgagactctccaggtttggtaagaaatattttatattgttataatgttactatgatccattaacactctgcttatagattgtaagggtgattgcaatgctttctgcataaaactttggttttcttgttaatcaataaaccgacttgattcgagaacctactcatatattattgtctcttttatactttattaagtaaaaggatttgtatattagccttgctaagggagacatctagtgatataagtgtgaactacacttatcttaaatgatgtaactccttaggataatcaatatacaaaattccatgacaattggcgcccaacgtggggctcgaatataagtcgggtttatttgtaaattatccctagggacctccgagcaagcgagcatagcgggaggcatataaaagccaatagacaatggctagcaggaagtaatgttgaagaatatgaacttgatgttgaagctctggttgtaattttaagagatagaaatataccaagaaatcctttacatggagaagttataggtcttcgccttactgaaggatggtggggacaaattgagagatttcagatggtacgttgatctaaggctatggatttggccatgggacaagaaatattagtttatagtcccattgtatctatgactaaaatacaaaaaactccactaccagaaagaaaagctttacccattagatggataacatggatgacttatttagaagatccaagaatccaatttcattatgataaaaccttaccagaacttaagcatattccagatgtatatacatctagtcagtctcctgttaaacatccttctcaatatgaaggagtgttttatactgatggctcggccatcaaaagtcctgatcctacaaaaagcaataatgctggcatgggaatagtacatgccacatacaaacctgaatatcaagttttgaatcaatggtcaataccactaggtaatcatactgctcagatggctgaaatagctgcagttgaatttgcctgtaaaaaagctttaaaaatacctggtcctgtattagttataactgatagtttctatgtagcagaaagtgctaataaagaattaccatactggaaatctaatgggtttgttaataataagaaaaagcctcttaaacatatctccaaatggaaatctattgctgagtgtttatctatgaaaccagacattactattcaacatgaaaaaggcatcagcctacaaataccagtattcatactgaaaggcaatgccctagcagataagcttgccacccaaggaagttatgtggttaattgtaataccaaaaaaccaaacctggatgcagagttggatcaattattacagggtcattatataaaaggatatcccaaacaatatacatattttttagaagatggcaaagtaaaagtttccagacctgaaggggttaaaattattccccctcagtcagacagacaaaaaattgtgcttcaagcccacaatttggctcacaccggacgtgaagccactcttttaaaaattgccaacctttattggtggccaaatatgagaaaggatgtggttaaacaactaggacgctgtcaacagtgtttaatcacaaatgcttccaacaaagcctctggtcctattctaagaccagataggcctcaaaaaccttttgataaattctttattgactatattggacctttgccaccttcacagggatacctatatgtattagtagttgttgatggaatgacaggattcacttggttataccccactaaggctccttctactagcgcaactgttaaatctctcaatgtactcactagtattgcaattccaaaggtgattcactctgatcaaggtgcagcattcacttcttcaacctttgctgaatgggcaaaggaaagaggtatacatttggaattcagtactccttatcacccccaaagtggtagtaaggtggaaaggaaaaatagtgatataaaacgacttttaactaaactgctagtaggaagacccacaaagtggtatgacctattgcctgttgtacaacttgctttaaacaacacctatagccctgtattaaaatatactccacatcaactcttatttggtatagattcaaatactccatttgcaaatcaagatacacttgacttgaccagagaagaagaactttctcttttacaggaaattcgtacttctttataccatccatccacccctccagcctcctctcgttcctggtctcctgttgttggccaattggtccaggagagggtggctaggcctgcttctttgagacctcgttggcataaaccgtctactgtacttaaggtgttgaatccaaggactgttgttattttggaccatcttggcaacaacagaactgtaagtatagataatttaaaacctacttctcatcagaatggcaccaccaatgacactgcaacaatggatcatttggaaaaaaatgaataaagcgcatgaggcacttcaaaatacaacaactgtgactgaacagcagaaggaacaaattatactggacattcaaaatgaagaagtacaaccaactaggagagataaatttagatatctgctttatacttgttgtgctactagctcaagagtattggcctggatgtttttagtttgtatattgttaatcattgttttggtttcatgctttgtgactatatccagaatacaatggaataaggatattcaggtattaggacctgtaatagactggaatgttactcaaagagctgtttatcaacccttacagactagaaggattgcacgttcccttagaatgcagcatcctgttccaaaatatgtggaggtaaatatgactagtattccacaaggtgtatactatgaaccccatccggcctcgacggtatcgatggtaccggtatcgataagcttgataacctcgagagatctaattctaccgggtaggggaggcgcttttcccaaggcagtctggagcatgcgctttagcagccccgctggcacttggcgctacacaagtggcctctggcctcgcacacattccacatccaccggtagcgccaaccggctccgttctttggtggccccttcgcgccacttctactcctcccctagtcaggaagtttcccccagcaagctcgcgtcgtgcaggacgtgacaaatggaagtagcacgtctcactagtctcgtgcagatggacagcaccgctgagcaatggaagcgggtaggcctttggggcagcggccaatagcagctttgttccttcgctttctgggctcagaggctgggaaggggtgggtccgggggcgggctcaggggcgggctcaggggcgggcgggcgcccgaaggtcctcccgaggcccggcattctgcacgcttcaaaagcgcacgtctgccgcgctgttctcctcttcctcatctccgggcctttcgacctgcagcccgggggatcgatcctaggtgatttaaatccaccatgaccgagtacaagcccacggtgcgcctcgccacccgcgacgacgtcccccgggccgtacgcaccctcgccgccgcgttcgccgactaccccgccacgcgccacaccgtcgacccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgagtgcccgaaggaccgcgcgacctggtgcatgacccgcaagcccggtgcctgacggatccatcgctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgcggccgcgccgccaccatgtctcgctccgtggccttagctgtgctcgcgctactctctctttctggcctggaggctatccagcgtactccaaagattcaggtttactcacgtcatccagcagagaatggaaagtcaaatttcctgaattgctatgtgtctgggtttcatccatccgacattgaagttgacttactgaagaatggagagagaattgaaaaagtggagcattcagacttgtctttcagcaaggactggtctttctatctcttgtactacactgaattcacccccactgaaaaagatgagtatgcctgccgtgtgaaccatgtgactttgtcacagcccaagatagttaagtgggatcgagacatgggcggaggcggaagcgggggcggcggatccggcggaggcggaagcggcggcgggggaagcggctctcactccatgaggtatttcttcacatccgtgtcccggcccggccgcggggagccccgcttcatcgcagtgggctacgtggacgacacgcagttcgtgcggttcgacagcgacgccgcgagccagaggatggagccgcgggcgccgtggatagagcaggagggtccggagtattgggacggggagacacggaaagtgaaggcccactcacagactcaccgagtggacctggggaccctgcgcggctactacaaccagagcgaggccggttctcacaccgtccagaggatgtatggctgcgacgtggggtcggactggcgcttcctccgcgggtaccaccagtacgcctacgacggcaaggattacatcgccctgaaagaggacctgcgctcttggaccgcggcggacatggcagctcagaccaccaagcacaagtgggaggcggcccatgtggcggagcagttgagagcctacctggagggcacgtgcgtggagtggctccgcagatacctggagaacgggaaggagacgctgcagcgcacggacgcccccaaaacgcatatgactcaccacgctgtctctgaccatgaagccaccctgaggtgctgggccctgagcttctaccctgcggagatcacactgacctggcagcgggatggggaggaccagacccaggacacggagctcgtggagaccaggcctgcaggggatggaaccttccagaagtgggcggctgtggtggtgccttctggacaggagcagagatacacctgccatgtgcagcatgagggtttgcccaagcccctcaccctgagatgggagccgtcttcccagcccaccatccccatcgtgggcatcattgctggcctggttctctttggagctgtgatcactggagctgtggtcgctgctgtgatgtggaggaggaagagctcagatagaaaaggagggagctactctcaggctgcaagcagtgacagtgcccagggctctgatgtgtctctcacagcttgtaaagtgtgagcggccgcgactctagatcaggcgcgccgttaccaagcagctatggaagcttatggacctcagagaggaagtaacgaggagagggtgtggtggaatgtcactagaaaccagggaaaacaaggaggagagtattacagggaaggaggtgaagaacctcattacccaaatactcctgctcctcatagacgtacctgggatgagagacacaaggttcttaaattgtcctcattcgctactccctctgacatccaacgctgggctactaactctagattgtacgggagctctcttcactactcgctgcgtcgagagtgtacgagactctccaggtttggtaagaaatattttatattgttataatgttactatgatccattaacactctgcttatagattgtaagggtgattgcaatgctttctgcataaaactttggttttcttgttaatcaataaaccgacttgattcgagaacctactcatatattattgtctcttttatactttattaagtaaaaggatttgtatattagccttgctaagggagacatctagtgatataagtgtgaactacacttatcttaaatgatgtaactccttaggataatcaatatacaaaattccatgacaattggcgataccgtcgaccgttcagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 16 - protein, 480 aa, synthetic construct
  REGION 1..480: note = HLA-bBA0201 (human B2M-HLA-A0201 fusion peptide)
MSRSVALAVLALLSLSGLEAIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGGGGSGSHSMRYFFTSVSRPGRGEPRFIAVGYVDDTQFVRFDSDAASQRMEPRAPWIEQEGPEYWDGETRKVKAHSQTHRVDLGTLRGYYNQSEAGSHTVQRMYGCDVGSDWRFLRGYHQYAYDGKDYIALKEDLRSWTAADMAAQTTKHKWEAAHVAEQLRAYLEGTCVEWLRRYLENGKETLQRTDAPKTHMTHHAVSDHEATLRCWALSFYPAEITLTWQRDGEDQTQDTELVETRPAGDGTFQKWAAVVVPSGQEQRYTCHVQHEGLPKPLTLRWEPSSQPTIPIVGIIAGLVLFGAVITGAVVAAVMWRRKSSDRKGGSYSQAASSDSAQGSDVSLTACKV

SEQ ID NO: 17 - DNA, 9224 bp, synthetic construct
  misc_feature 1..9224: note = Vector sequence for HLA-gBE
  source 1..9224: mol_type = other DNA
ctcgaggagcttggcccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacgctaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagcttctagattgtacgggagctcttcactactcgctgcgtcgagagtgtacgagactctccaggtttggtaagaaatattttatattgttataatgttactatgatccattaacactctgcttatagattgtaagggtgattgcaatgctttctgcataaaactttggttttcttgttaatcaataaaccgacttgattcgagaacctactcatatattattgtctcttttatactttattaagtaaaaggatttgtatattagccttgctaagggagacatctagtgatataagtgtgaactacacttatcttaaatgatgtaactccttaggataatcaatatacaaaattccatgacaattggcgcccaacgtggggctcgaatataagtcgggtttatttgtaaattatccctagggacctccgagcatagcgggaggcatataaaagccaatagacaatggctagcaggaagtaatgttgaagaatatgaacttgatgttgaagctctggttgtaattttaagagatagaaatataccaagaaatcctttacatggagaagttataggtcttcgccttactgaaggatggtggggacaaattgagagatttcagatggtacgttgatctaaggctatggatttggccatgggacaagaaatattagtttatagtcccattgtatctatgactaaaatacaaaaaactccactaccagaaagaaaagctttacccattagatggataacatggatgacttatttagaagatccaagaatccaatttcattatgataaaaccttaccagaacttaagcatattccagatgtatatacatctagtcagtctcctgttaaacatccttctcaatatgaaggagtgttttatactgatggctcggccatcaaaagtcctgatcctacaaaaagcaataatgctggcatgggaatagtacatgccacatacaaacctgaatatcaagttttgaatcaatggtcaataccactaggtaatcatactgctcagatggctgaaatagctgcagttgaatttgcctgtaaaaaagctttaaaaatacctggtcctgtattagttataactgatagtttctatgtagcagaaagtgctaataaagaattaccatactggaaatctaatgggtttgttaataataagaaaaagcctcttaaacatatctccaaatggaaatctattgctgagtgtttatctatgaaaccagacattactattcaacatgaaaaaggcatcagcctacaaataccagtattcatactgaaaggcaatgccctagcagataagcttgccacccaaggaagttatgtggttaattgtaataccaaaaaaccaaacctggatgcagagttggatcaattattacagggtcattatataaaaggatatcccaaacaatatacatattttttagaagatggcaaagtaaaagtttccagacctgaaggggttaaaattattccccctcagtcagacagacaaaaaattgtgcttcaagcccacaatttggctcacaccggacgtgaagccactcttttaaaaattgccaacctttattggtggccaaatatgagaaaggatgtggttaaacaactaggacgctgtcaacagtgtttaatcacaaatgcttccaacaaagcctctggtcctattctaagaccagataggcctcaaaaaccttttgataaattctttattgactatattggacctttgccaccttcacagggatacctatatgtattagtagttgttgatggaatgacaggattcacttggttataccccactaaggctccttctactagcgcaactgttaaatctctcaatgtactcactagtattgcaattccaaaggtgattcactctgatcaaggtgcagcattcacttcttcaacctttgctgaatgggcaaaggaaagaggtatacatttggaattcagtactccttatcacccccaaagtggtagtaaggtggaaaggaaaaatagtgatataaaacgacttttaactaaactgctagtaggaagacccacaaagtggtatgacctattgcctgttgtacaacttgctttaaacaacacctatagccctgtattaaaatatactccacatcaactcttatttggtatagattcaaatactccatttgcaaatcaagatacacttgacttgaccagagaagaagaactttctcttttacaggaaattcgtacttctttataccatccatccacccctccagcctcctctcgttcctggtctcctgttgttggccaattggtccaggagagggtggctaggcctgcttctttgagacctcgttggcataaaccgtctactgtacttaaggtgttgaatccaaggactgttgttattttggaccatcttggcaacaacagaactgtaagtatagataatttaaaacctacttctcatcagaatggcaccaccaatgacactgcaacaatggatcatttggaaaaaaatgaataaagcgcatgaggcacttcaaaatacaacaactgtgactgaacagcagaaggaacaaattatactggacattcaaaatgaagaagtacaaccaactaggagagataaatttagatatctgctttatacttgttgtgctactagctcaagagtattggcctggatgtttttagtttgtatattgttaatcattgttttggtttcatgctttgtgactatatccagaatacaatggaataaggatattcaggtattaggacctgtaatagactggaatgttactcaaagagctgtttatcaacccttacagactagaaggattgcacgttcccttagaatgcagcatcctgttccaaaatatgtggaggtaaatatgactagtattccacaaggtgtatactatgaaccccatccggcctcgacggtatcgatggtaccggtatcgataagcttgataacctcgagagatctaattctaccgggtaggggaggcgcttttcccaaggcagtctggagcatgcgctttagcagccccgctggcacttggcgctacacaagtggcctctggcctcgcacacattccacatccaccggtagcgccaaccggctccgttctttggtggccccttcgcgccacttctactcctcccctagtcaggaagtttcccccagcaagctcgcgtcgtgcaggacgtgacaaatggaagtagcacgtctcactagtctcgtgcagatggacagcaccgctgagcaatggaagcgggtaggcctttggggcagcggccaatagcagctttgttccttcgctttctgggctcagaggctgggaaggggtgggtccgggggcgggctcaggggcgggctcaggggcgggcgggcgcccgaaggtcctcccgaggcccggcattctgcacgcttcaaaagcgcacgtctgccgcgctgttctcctcttcctcatctccgggcctttcgacctgcagcccgggggatcgatcctaggtgatttaaatccaccatgaccgagtacaagcccacggtgcgcctcgccacccgcgacgacgtcccccgggccgtacgcaccctcgccgccgcgttcgccgactaccccgccacgcgccacaccgtcgacccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgagtgcccgaaggaccgcgcgacctggtgcatgacccgcaagcccggtgcctgacggatccatcgctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgcggccgcgttaaccatggtggtcatggcccctcgaactctgttcctgctgctgagtggggctctgaccctgacagagtccggaatccagcggacccccaagattcaggtgtacagcagacaccctgcagagaacggcaaatccaacttcctgaattgctatgtgtctgggtttcatcccagtgacatcgaagtcgatctgctgaagaatggcgagagaattgaaaaagtcgagcactctgacctgagcttctccaaggattggtccttttacctgctgtactataccgagtttactccaaccgaaaaagacgagtatgcctgtagggtgaaccatgtcacactgagccagcccaagatcgtgaaatgggaccgcgatatgggcgggggaggctcagggggaggcgggagcggaggcgggggctccggcgggggaggatccggttctcactccttgaagtatttccacacttccgtgtcccggcccggccgcggggagccccgcttcatctctgtgggctacgtggacgacacccagttcgtgcgcttcgacaacgacgccgcgagtccgaggatggtgccgcgggcgccgtggatggagcaggaggggtcagagtattgggaccgggagacacggagcgccagggacaccgcacagattttccgagtgaatctgcggacgctgcgcggctactacaatcagagcgaggccgggtctcacaccctgcagtggatgcatggctgcgagctggggcccgacgggcgcttcctccgcgggtatgaacagttcgcctacgacggcaaggattatctcaccctgaatgaggacctgcgctcctggaccgcggtggacacggcggctcagatctccgagcaaaagtcaaatgatgcctctgaggcggagcaccagagagcctacctggaagacacatgcgtggagtggctccacaaatacctggagaaggggaaggagacgctgcttcacctggagcccccaaagacacacgtgactcaccaccccatctctgaccatgaggccaccctgaggtgctgggccctgggcttctaccctgcggagatcacactgacctggcagcaggatggggagggccatacccaggacacggagctcgtggagaccaggcctgcaggggatggaaccttccagaagtgggcagctgtggtggtgccttctggagaggagcagagatacacgtgccatgtgcagcatgaggggctacccgagcccgtcaccctgagatggaagccggcttcccagcccaccatccccatcgtgggcatcattgctggcctggttctccttggatctgtggtctctggagctgtggttgctgctgtgatatggaggaagaagagctcaggtggaaaaggagggagctactctaaggctgagtggagcgacagtgcccaggggtctgagtctcacagcttgtaatctagagcggccgcgactctagatcaggcgcgccgttaccaagcagctatggaagcttatggacctcagagaggaagtaacgaggagagggtgtggtggaatgtcactagaaaccagggaaaacaaggaggagagtattacagggaaggaggtgaagaacctcattacccaaatactcctgctcctcatagacgtacctgggatgagagacacaaggttcttaaattgtcctcattcgctactccctctgacatccaacgctggactactaactctagattgtacgggaggctcttcactactcgctgcgtcgagagtgtacgagactctccaggtttggtaagaaatattttatattgttataatgttactatgatccattaacactctgcttatagattgtaagggtgattgcaatgctttctgcataaaactttggttttcttgttaatcaataaaccgacttgattcgagaaccaactcctatattattgtctcttttatactttattaagtaaaaggatttgtatattagccttgctaagggagacatctagtgatataagtgtgaactacacttatcttaaatgatgtaactccttaggataatcaatatacaaaattccatgacaattggcgataccgtcgaccgttcagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 18 - protein, 476 aa, synthetic construct
  REGION 1..476: note = HLA-gBE
MAPRTLFLLLSGALTLTESGIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGGGGSGSHSLKYFHTSVSRPGRGEPRFISVGYVDDTQFVRFDNDAASPRMVPRAPWMEQEGSEYWDRETRSARDTAQIFRVNLRTLRGYYNQSEAGSHTLQWMHGCELGPDGRFLRGYEQFAYDGKDYLTLNEDLRSWTAVDTAAQISEQKSNDASEAEHQRAYLEDTCVEWLHKYLEKGKETLLHLEPPKTHVTHHPISDHEATLRCWALGFYPAEITLTWQQDGEGHTQDTELVETRPAGDGTFQKWAAVVVPSGEEQRYTCHVQHEGLPEPVTLRWKPASQPTIPIVGIIAGLVLLGSVVSGAVVAAVIWRKKSSGGKGGSYSKAEWSDSAQGSESHSL

SEQ ID NO: 19 - DNA, 9287 bp, synthetic construct
  misc_feature 1..9287: note = Vector sequence for HLA-bGBE
  source 1..9287: mol_type = other DNA
ctcgaggagcttggcccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacgctaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagcttctagattgtacgggagctcttcactactcgctgcgtcgagagtgtacgagactctccaggtttggtaagaaatattttatattgttataatgttactatgatccattaacactctgcttatagattgtaagggtgattgcaatgctttctgcataaaactttggttttcttgttaatcaataaaccgacttgattcgagaacctactcatatattattgtctcttttatactttattaagtaaaaggatttgtatattagccttgctaagggagacatctagtgatataagtgtgaactacacttatcttaaatgatgtaactccttaggataatcaatatacaaaattccatgacaattggcgcccaacgtggggctcgaatataagtcgggtttatttgtaaattatccctagggacctccgagcatagcgggaggcatataaaagccaatagacaatggctagcaggaagtaatgttgaagaatatgaacttgatgttgaagctctggttgtaattttaagagatagaaatataccaagaaatcctttacatggagaagttataggtcttcgccttactgaaggatggtggggacaaattgagagatttcagatggtacgttgatctaaggctatggatttggccatgggacaagaaatattagtttatagtcccattgtatctatgactaaaatacaaaaaactccactaccagaaagaaaagctttacccattagatggataacatggatgacttatttagaagatccaagaatccaatttcattatgataaaaccttaccagaacttaagcatattccagatgtatatacatctagtcagtctcctgttaaacatccttctcaatatgaaggagtgttttatactgatggctcggccatcaaaagtcctgatcctacaaaaagcaataatgctggcatgggaatagtacatgccacatacaaacctgaatatcaagttttgaatcaatggtcaataccactaggtaatcatactgctcagatggctgaaatagctgcagttgaatttgcctgtaaaaaagctttaaaaatacctggtcctgtattagttataactgatagtttctatgtagcagaaagtgctaataaagaattaccatactggaaatctaatgggtttgttaataataagaaaaagcctcttaaacatatctccaaatggaaatctattgctgagtgtttatctatgaaaccagacattactattcaacatgaaaaaggcatcagcctacaaataccagtattcatactgaaaggcaatgccctagcagataagcttgccacccaaggaagttatgtggttaattgtaataccaaaaaaccaaacctggatgcagagttggatcaattattacagggtcattatataaaaggatatcccaaacaatatacatattttttagaagatggcaaagtaaaagtttccagacctgaaggggttaaaattattccccctcagtcagacagacaaaaaattgtgcttcaagcccacaatttggctcacaccggacgtgaagccactcttttaaaaattgccaacctttattggtggccaaatatgagaaaggatgtggttaaacaactaggacgctgtcaacagtgtttaatcacaaatgcttccaacaaagcctctggtcctattctaagaccagataggcctcaaaaaccttttgataaattctttattgactatattggacctttgccaccttcacagggatacctatatgtattagtagttgttgatggaatgacaggattcacttggttataccccactaaggctccttctactagcgcaactgttaaatctctcaatgtactcactagtattgcaattccaaaggtgattcactctgatcaaggtgcagcattcacttcttcaacctttgctgaatgggcaaaggaaagaggtatacatttggaattcagtactccttatcacccccaaagtggtagtaaggtggaaaggaaaaatagtgatataaaacgacttttaactaaactgctagtaggaagacccacaaagtggtatgacctattgcctgttgtacaacttgctttaaacaacacctatagccctgtattaaaatatactccacatcaactcttatttggtatagattcaaatactccatttgcaaatcaagatacacttgacttgaccagagaagaagaactttctcttttacaggaaattcgtacttctttataccatccatccacccctccagcctcctctcgttcctggtctcctgttgttggccaattggtccaggagagggtggctaggcctgcttctttgagacctcgttggcataaaccgtctactgtacttaaggtgttgaatccaaggactgttgttattttggaccatcttggcaacaacagaactgtaagtatagataatttaaaacctacttctcatcagaatggcaccaccaatgacactgcaacaatggatcatttggaaaaaaatgaataaagcgcatgaggcacttcaaaatacaacaactgtgactgaacagcagaaggaacaaattatactggacattcaaaatgaagaagtacaaccaactaggagagataaatttagatatctgctttatacttgttgtgctactagctcaagagtattggcctggatgtttttagtttgtatattgttaatcattgttttggtttcatgctttgtgactatatccagaatacaatggaataaggatattcaggtattaggacctgtaatagactggaatgttactcaaagagctgtttatcaacccttacagactagaaggattgcacgttcccttagaatgcagcatcctgttccaaaatatgtggaggtaaatatgactagtattccacaaggtgtatactatgaaccccatccggcctcgacggtatcgatggtaccggtatcgataagcttgataacctcgagagatctaattctaccgggtaggggaggcgcttttcccaaggcagtctggagcatgcgctttagcagccccgctggcacttggcgctacacaagtggcctctggcctcgcacacattccacatccaccggtagcgccaaccggctccgttctttggtggccccttcgcgccacttctactcctcccctagtcaggaagtttcccccagcaagctcgcgtcgtgcaggacgtgacaaatggaagtagcacgtctcactagtctcgtgcagatggacagcaccgctgagcaatggaagcgggtaggcctttggggcagcggccaatagcagctttgttccttcgctttctgggctcagaggctgggaaggggtgggtccgggggcgggctcaggggcgggctcaggggcgggcgggcgcccgaaggtcctcccgaggcccggcattctgcacgcttcaaaagcgcacgtctgccgcgctgttctcctcttcctcatctccgggcctttcgacctgcagcccgggggatcgatcctaggtgatttaaatccaccatgaccgagtacaagcccacggtgcgcctcgccacccgcgacgacgtcccccgggccgtacgcaccctcgccgccgcgttcgccgactaccccgccacgcgccacaccgtcgacccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgagtgcccgaaggaccgcgcgacctggtgcatgacccgcaagcccggtgcctgacggatccatcgctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgcggccgcgttaaccatgagccgatccgtggcactggctgtcctggctctgctgtctctgagtggcctggaagcagtgatggcccctagaacactgttcctgggcggaggcggctccggaggaggagggtctggaggcgggggaagtatccagcggactcccaagattcaggtctacagcagacaccctgccgaaaacgggaaatccaacttcctgaattgctatgtgtcaggctttcatcccagcgacatcgaggtcgatctgctgaagaatggcgagcggattgaaaaagtggagcactctgacctgtcattcagcaaggattggagcttttacctgctgtactatactgagtttaccccaacagaaaaagacgagtatgcctgtagggtgaaccatgtcaccctgagtcagcccaagatcgtgaaatgggaccgcgatatgggcgggggaggctccgggggaggcggctccggcggcgggggaagtggcgggggaggatccggttctcactccttgaagtatttccacacttccgtgtcccggcccggccgcggggagccccgcttcatctctgtgggctacgtggacgacacccagttcgtgcgcttcgacaacgacgccgcgagtccgaggatggtgccgcgggcgccgtggatggagcaggaggggtcagagtattgggaccgggagacacggagcgccagggacaccgcacagattttccgagtgaatctgcggacgctgcgcggctactacaatcagagcgaggccgggtctcacaccctgcagtggatgcatggctgcgagctggggcccgacgggcgcttcctccgcgggtatgaacagttcgcctacgacggcaaggattatctcaccctgaatgaggacctgcgctcctggaccgcggtggacacggcggctcagatctccgagcaaaagtcaaatgatgcctctgaggcggagcaccagagagcctacctggaagacacatgcgtggagtggctccacaaatacctggagaaggggaaggagacgctgcttcacctggagcccccaaagacacacgtgactcaccaccccatctctgaccatgaggccaccctgaggtgctgggccctgggcttctaccctgcggagatcacactgacctggcagcaggatggggagggccatacccaggacacggagctcgtggagaccaggcctgcaggggatggaaccttccagaagtgggcagctgtggtggtgccttctggagaggagcagagatacacgtgccatgtgcagcatgaggggctacccgagcccgtcaccctgagatggaagccggcttcccagcccaccatccccatcgtgggcatcattgctggcctggttctccttggatctgtggtctctggagctgtggttgctgctgtgatatggaggaagaagagctcaggtggaaaaggagggagctactctaaggctgagtggagcgacagtgcccaggggtctgagtctcacagcttgtaatctagagcggccgcgactctagatcaggcgcgccgttaccaagcagctatggaagcttatggacctcagagaggaagtaacgaggagagggtgtggtggaatgtcactagaaaccagggaaaacaaggaggagagtattacagggaaggaggtgaagaacctcattacccaaatactcctgctcctcatagacgtacctgggatgagagacacaaggttcttaaattgtcctcattcgctactccctctgacatccaacgctggactactaactctagattgtacgggaggctcttcactactcgctgcgtcgagagtgtacgagactctccaggtttggtaagaaatattttatattgttataatgttactatgatccattaacactctgcttatagattgtaagggtgattgcaatgctttctgcataaaactttggttttcttgttaatcaataaaccgacttgattcgagaaccaactcctatattattgtctcttttatactttattaagtaaaaggatttgtatattagccttgctaagggagacatctagtgatataagtgtgaactacacttatcttaaatgatgtaactccttaggataatcaatatacaaaattccatgacaattggcgataccgtcgaccgttcagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 20 - protein, 500 aa, synthetic construct
  REGION 1..500: note = HLA-bGBE
MSRSVALAVLALLSLSGLEAVMAPRTLFLGGGGSGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGGGGSGSHSLKYFHTSVSRPGRGEPRFISVGYVDDTQFVRFDNDAASPRMVPRAPWMEQEGSEYWDRETRSARDTAQIFRVNLRTLRGYYNQSEAGSHTLQWMHGCELGPDGRFLRGYEQFAYDGKDYLTLNEDLRSWTAVDTAAQISEQKSNDASEAEHQRAYLEDTCVEWLHKYLEKGKETLLHLEPPKTHVTHHPISDHEATLRCWALGFYPAEITLTWQQDGEGHTQDTELVETRPAGDGTFQKWAAVVVPSGEEQRYTCHVQHEGLPEPVTLRWKPASQPTIPIVGIIAGLVLLGSVVSGAVVAAVIWRKKSSGGKGGSYSKAEWSDSAQGSESHSL

SEQ ID NO: 21 - DNA, 7159 bp, synthetic construct
  misc_feature 1..7159: note = Vector sequence for pA2-B2METKNpA
  source 1..7159: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcgatgcagtccaaactctcactaaaattgccgagccctttgtcttccagtgtctaaaatattaatgtcaatggaatcaggccagagtttgaattctagtctcttagcctttgtttcccctgtccataaaatgaatgggggtaattctttcctcctacagtttatttatatattcactaattcattcattcatccatccattcgttcattcggtttactgagtacctactatgtgccagcccctgttctagggtggaaactaagagaatgatgtacctagagggcgctggaagctctaaagccctagcagttactgcttttactattagtggtcgtttttttctcccccccgccccccgacaaatcaacagaacaaagaaaattacctaaacagcaaggacatagggaggaacttcttggcacagaactttccaaacactttttcctgaagggatacaagaagcaagaaaggtactctttcactaggaccttctctgagctgtcctcaggatgcttttgggactatttttcttacccagagaatggagaaaccctgcagggaattcccaagctgtagttataaacagaagttctccttctgctaggtagcattcaaagatcttaatcttctgggtttccgttttctcgaatgaaaaatgcaggtccgagcagttaactggctggggcaccattagcaagtcacttagcatctctggggccagtctgcaaagcgagggggcagccttaatgtgcctccagcctgaagtcctagaatgagcgcccggtgtcccaagctggggcgcgcaccccagatcggagggcgccgatgtacagacagcaaactcacccagtctagtgcatgccttcttaaacatcacgagactctaagaaaaggaaactgaaaacgggaaagtccctctctctaacctggcactgcgtcgctggcttggagacaggtgacggtccctgcgggccttgtcctgattggctgggcacgcgtttaatataagtggaggcgtcgcgctggcgggcattcctgaagctgacagcattcgggccgagatctagataacttcgtataatgtatgctatacgaagttatggatccatcgattctgcgatcgctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttccaccatgcccacgctactgcgggtttatatagacggtccccacgggatggggaaaaccaccaccacgcaactgctggtggccctgggttcgcgcgacgatatcgtctacgtacccgagccgatgacttactggcgggtgctgggggcttccgagacaatcgcgaacatctacaccacacaacaccgcctcgaccagggtgagatatcggccggggacgcggcggtggtaatgacaagcgcccagataacaatgggcatgccttatgccgtgaccgacgccgttctggctcctcatatcgggggggaggctgggagctcacatgccccgcccccggccctcaccctcatcttcgaccgccatcccatcgccgccctcctgtgctacccggccgcgcggtaccttatgggcagcatgaccccccaggccgtgctggcgttcgtggccctcatcccgccgaccttgcccggcaccaacatcgtgcttggggcccttccggaggacagacacatcgaccgcctggccaaacgccagcgccccggcgagcggctggacctggctatgctggctgcgattcgccgcgtttacgggctacttgccaatacggtgcggtatctgcagtgcggcgggtcgtggcgggaggactggggacagctttcggggacggccgtgccgccccagggtgccgagccccagagcaacgcgggcccacgaccccatatcggggacacgttatttaccctgtttcgggcccccgagttgctggcccccaacggcgacctgtataacgtgtttgcctgggccttggacgtcttggccaaacgcctccgttccatgcacgtctttatcctggattacgaccaatcgcccgccggctgccgggacgccctgctgcaacttacctccgggatggtccagacccacgtcaccacccccggctccataccgacgatatgcgacctggcgcgcacgtttgcccgggagatgggatcggccattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagggtctctagcataacttcgtataatgtatgctatacgaagttataataaaagatccttattttcattggatctgtgtgttggttttttgtgtgggatccgtcgataccgtcgactgtctcgctccgtggccttagctgtgctcgcgctactctctctttctggcctggaggctatccagcgtgagtctctcctaccctcccgctctggtccttcctctcccgctctgcaccctctgtggccctcgctgtgctctctcgctccgtgacttcccttctccaagttctccttggtggcccgccgtggggctagtccagggctggatctcggggaagcggcggggtggcctgggagtggggaagggggtgcgcacccgggacgcgcgctacttgcccctttcggcggggagcaggggagacctttggcctacggcgacgggagggtcgggacaaagtttagggcgtcgataagcgtcagagcgccgaggttgggggagggtttctcttccgctctttcgcggggcctctggctcccccagcgcagctggagtgggggacgggtaggctcgtcccaaaggcgcggcgctgaggtttgtgaacgcgtggaggggcgcttggggtctgggggaggcgtcgcccgggtaagcctgtctgctgcggctctgcttcccttagactggagagctgtggacttcgtctaggcgcccgctaagttcgcatgtcctagcacctctgggtctatgtggggccacaccgtggggaggaaacagcacgcgacgtttgtagaatgcttggctgtgatacaaagcggtttcgaataattaacttatttgttcccatcacatgtcacttttaaaaaattataagaactacccgttattgacatctttctgtgtgccaaggactttatgtgctttgcgtcatttaattttgaaaacagttatcttccgccatagataactactatggttatcttctgcctctcacagatgaagaaactaaggcaccgagattttaagaaacttaattacacaggggataaatggcagcaatcgagattgaagtcaagcctaaccagggcttttgcgggagcgcatgccttttggctgtaattcgtgcgcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgtcctgcaggcag

SEQ ID NO: 22 - DNA, 7199 bp, synthetic construct
  misc_feature 1..7199: note = Vector sequence forpA2-B2MEHyTKpA
  source 1..7199: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcgaaactaagagaatgatgtacctagagggcgctggaagctctaaagccctagcagttactgcttttactattagtggtcgtttttttctcccccccgccccccgacaaatcaacagaacaaagaaaattacctaaacagcaaggacatagggaggaacttcttggcacagaactttccaaacactttttcctgaagggatacaagaagcaagaaaggtactctttcactaggaccttctctgagctgtcctcaggatgcttttgggactatttttcttacccagagaatggagaaaccctgcagggaattcccaagctgtagttataaacagaagttctccttctgctaggtagcattcaaagatcttaatcttctgggtttccgttttctcgaatgaaaaatgcaggtccgagcagttaactggctggggcaccattagcaagtcacttagcatctctggggccagtctgcaaagcgagggggcagccttaatgtgcctccagcctgaagtcctagaatgagcgcccggtgtcccaagctggggcgcgcaccccagatcggagggcgccgatgtacagacagcaaactcacccagtctagtgcatgccttcttaaacatcacgagactctaagaaaaggaaactgaaaacgggaaagtccctctctctaacctggcactgcgtcgctggcttggagacaggtgacggtccctgcgggccttgtcctgattggctgggcacgcgtttaatataagtggaggcgtcgcgctggcgggcattcctgaagctgacagcattcgggccgagatctagataacttcgtataatgtatgctatacgaagttatggatccatcgattctgcgatcgctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttccaccatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtcgcgtctgcgttcgaccaggctgcgcgttctcgcggccatagcaaccgacgtacggcgttgcgccctcgccggcagcaagaagccacggaagtccgcccggagcagaaaatgcccacgctactgcgggtttatatagacggtccccacgggatggggaaaaccaccaccacgcaactgctggtggccctgggttcgcgcgacgatatcgtctacgtacccgagccgatgacttactggcgggtgctgggggcttccgagacaatcgcgaacatctacaccacacaacaccgcctcgaccagggtgagatatcggccggggacgcggcggtggtaatgacaagcgcccagataacaatgggcatgccttatgccgtgaccgacgccgttctggctcctcatatcgggggggaggctgggagctcacatgccccgcccccggccctcaccctcatcttcgaccgccatcccatcgccgccctcctgtgctacccggccgcgcggtaccttatgggcagcatgaccccccaggccgtgctggcgttcgtggccctcatcccgccgaccttgcccggcaccaacatcgtgcttggggcccttccggaggacagacacatcgaccgcctggccaaacgccagcgccccggcgagcggctggacctggctatgctggctgcgattcgccgcgtttacgggctacttgccaatacggtgcggtatctgcagtgcggcgggtcgtggcgggaggactggggacagctttcggggacggccgtgccgccccagggtgccgagccccagagcaacgcgggcccacgaccccatatcggggacacgttatttaccctgtttcgggcccccgagttgctggcccccaacggcgacctgtataacgtgtttgcctgggccttggacgtcttggccaaacgcctccgttccatgcacgtctttatcctggattacgaccaatcgcccgccggctgccgggacgccctgctgcaacttacctccgggatggtccagacccacgtcaccacccccggctccataccgacgatatgcgacctggcgcgcacgtttgcccgggagatgggggaggctaactgaggtctctagcataacttcgtataatgtatgctatacgaagttataataaaagatccttattttcattggatctgtgtgttggttttttgtgtgggatccgtcgataccgtcgactgtctcgctccgtggccttagctgtgctcgcgctactctctctttctggcctggaggctatccagcgtgagtctctcctaccctcccgctctggtccttcctctcccgctctgcaccctctgtggccctcgctgtgctctctcgctccgtgacttcccttctccaagttctccttggtggcccgccgtggggctagtccagggctggatctcggggaagcggcggggtggcctgggagtggggaagggggtgcgcacccgggacgcgcgctacttgcccctttcggcggggagcaggggagacctttggcctacggcgacgggagggtcgggacaaagtttagggcgtcgataagcgtcagagcgccgaggttgggggagggtttctcttccgctctttcgcggggcctctggctcccccagcgcagctggagtgggggacgggtaggctcgtcccaaaggcgcggcgctgaggtttgtgaacgcgtggaggggcgcttggggtctgggggaggcgtcgcccgggtaagcctgtctgctgcggctctgcttcccttagactggagagctgtggacttcgtctaggcgcccgctaagttcgcatgtcctagcacctctgggtctatgtggggccacaccgtggggaggaaacagcacgcgacgtttgtagaatgcttggctgtgatacaaagcggtttcgaataattaacttatttgttcccatcacatgtcacttttaaaaaattataagaactacccgttattgacatctttctgtgtgccaaggactttatgtgctttgcgtcatttaattttgaaaacagttatcttccgccatagataactactatggttatcttctgcctctcacagatgaagaaactaaggcaccgagattttaagaaacttaattacacaggggataaatggcagcaatcgagattgaagtcaagcctaaccagggcttttgcgggagcgcatgccttttggctgtaattcgtgcgcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 23 - protein, 29 aa, synthetic construct
  REGION 1..29: note = HLA-G peptide
MAPRTLFLGGGGSGGGGSGGGGSIQRTPK

SEQ ID NO: 24 - DNA, 1369 bp, Homo sapiens
  source 1..1369: mol_type = genomic DNA
  CDS 506..1219
ccggacgccgcacggctcctgttccggtgtcagagggcccgccctccccgctcctcagtctttgcggacaagaaaggggctgtgtgagacgcagggaaggaggcacacccgggggtggcgcagtgaggagggggcgcgacggccaggaggctggtggagcgacacccaggcaggagagggggaagaactctctccctttctgaacccccttttccttgagagacgagttgggggagtcctccacgcattacccactcgggccgcaaaaactcccttctttagccctctgcccccgcccttgcttataagcctttgagaccgcagaagggaccttgttgtggaacgggacggccaagaggaagccagatcgctgagggtccggtctccagtttgcctcctgctatatccattggaagagaaaagtttgtgacttgggcccccaagttttgagagaactgggctttcggcgcggggggacagaggaggctcgtggggagctttccccatggagcttacccagcctgcagaagacctcatccagacccagcagacccctgcctcagaacttggggaccctgaagaccccggagaggaggctgcagatggctcagacactgtggtcctcagtctctttccctgcacccctgagcctgtgaatcctgaaccggatgccagtgtttcctctccacagggcagctccctgaagcactccaccactctcaccaaccggcagcgagggaacgaggtgtcagctctgccggccaccctagactgtgacaacctcgtcaacaagccagacgagcgcggcttcacccccctcatctgggcctccgcctttggagagattgagaccgttcgcttcctgctggagtggggtgccgacccccacatcctggcaaaagagcgagagagcgccctgtcgctggccagcacaggcggctacacagacattgtggggctgctgctggagcgtgacgtggacatcaacatctatgattggaatggagggacgccactgctgtacgctgtgcgcgggaaccacgtgaaatgcgttgaggccttgctggcccgaggcgctgacctcaccaccgaagccgactctggctacaccccgatggaccttgccgtggccctgggataccggaaagtgcaacaggtgatcgagaaccacatcctcaagctcttccagagcaacctggtgcccgctgaccctgagtgaaggccgcctgccggggactcagacactcagggaacaaaatggtcagccagagctggggaaacccagaactgacttcaaaggcagcttctggacaggtggtgggaggggacccttcccaagaggaaccaataaaccttctgtgcagaatga

SEQ ID NO: 25 - protein, 237 aa, Homo sapiens
MELTQPAEDLIQTQQTPASELGDPEDPGEEAADGSDTVVLSLFPCTPEPVNPEPDASVSSPQGSSLKHSTTLTNRQRGNEVSALPATLDCDNLVNKPDERGFTPLIWASAFGEIETVRFLLEWGADPHILAKERESALSLASTGGYTDIVGLLLERDVDINIYDWNGGTPLLYAVRGNHVKCVEALLARGADLTTEADSGYTPMDLAVALGYRKVQQVIENHILKLFQSNLVPADPE

SEQ ID NO: 26 - DNA, 1438 bp, Homo sapiens
  source 1..1438: mol_type = genomic DNA
  CDS 506..1288
ccggacgccgcacggctcctgttccggtgtcagagggcccgccctccccgctcctcagtctttgcggacaagaaaggggctgtgtgagacgcagggaaggaggcacacccgggggtggcgcagtgaggagggggcgcgacggccaggaggctggtggagcgacacccaggcaggagagggggaagaactctctccctttctgaacccccttttccttgagagacgagttgggggagtcctccacgcattacccactcgggccgcaaaaactcccttctttagccctctgcccccgcccttgcttataagcctttgagaccgcagaagggaccttgttgtggaacgggacggccaagaggaagccagatcgctgagggtccggtctccagtttgcctcctgctatatccattggaagagaaaagtttgtgacttgggcccccaagttttgagagaactgggctttcggcgcggggggacagaggaggctcgtggggagctttccccatggagcttacccagcctgcagaagacctcatccagacccagcagacccctgcctcagaacttggggaccctgaagaccccggagaggaggctgcagatggctcagacactgtggtcctcagtctctttccctgcacccctgagcctgtgaatcctgaaccggatgccagtgtttcctctccacaggcaggcagctccctgaagcactccaccactctcaccaaccggcagcgagggaacgaggtgtcagctctgccggccaccctagactccctgtccatccaccagctcgcagcacagggggagctggaccagctgaaggagcatttgcggaaaggtgacaacctcgtcaacaagccagacgagcgcggcttcacccccctcatctgggcctccgcctttggagagattgagaccgttcgcttcctgctggagtggggtgccgacccccacatcctggcaaaagagcgagagagcgccctgtcgctggccagcacaggcggctacacagacattgtggggctgctgctggagcgtgacgtggacatcaacatctatgattggaatggagggacgccactgctgtacgctgtgcgcgggaaccacgtgaaatgcgttgaggccttgctggcccgaggcgctgacctcaccaccgaagccgactctggctacaccccgatggaccttgccgtggccctgggataccggaaagtgcaacaggtgatcgagaaccacatcctcaagctcttccagagcaacctggtgcccgctgaccctgagtgaaggccgcctgccggggactcagacactcagggaacaaaatggtcagccagagctggggaaacccagaactgacttcaaaggcagcttctggacaggtggtgggaggggacccttcccaagaggaaccaataaaccttctgtgcagaatga

SEQ ID NO: 27 - protein, 260 aa, Homo sapiens
MELTQPAEDLIQTQQTPASELGDPEDPGEEAADGSDTVVLSLFPCTPEPVNPEPDASVSSPQAGSSLKHSTTLTNRQRGNEVSALPATLDSLSIHQLAAQGELDQLKEHLRKGDNLVNKPDERGFTPLIWASAFGEIETVRFLLEWGADPHILAKERESALSLASTGGYTDIVGLLLERDVDINIYDWNGGTPLLYAVRGNHVKCVEALLARGADLTTEADSGYTPMDLAVALGYRKVQQVIENHILKLFQSNLVPADPE

SEQ ID NO: 28 - DNA, 3618 bp, Homo sapiens
  source 1..3618: mol_type = genomic DNA
  CDS 222..2072
attcagaaaaaggaaaataaagcaacgcacgagccggctgggccaggaccttggggctgggctggagtgaaggtggctacgagttttccagatttaggagacttcagaaaggtggggcagatagaatggagatggcaaagatctctttgggcatatatgggcctggcgaagtaatggaataatttctaattttcggagaaggcaagtgccctcatgccgggatggcagaagatgagcctgatgctaagagccccaagactgggggaagggcccccccaggtggtgctgaggctggggaacctaccacccttcttcagaggctccgaggtaccatttccaaggccgtgcagaacaaagtagaggggatcctgcaagatgtacagaaattttctgacaatgacaagctgtatctctaccttcagctcccctcaggacccaccactggagacaaaagctcagagccaagtacactgagcaatgaggagtacatgtatgcctataggtggatccgcaaccacctggaagagcacactgacacctgtctgccaaagcaaagtgtttatgatgcctatcggaagtactgtgagagtcttgcctgttgccgcccactcagcacagccaactttggcaagatcatcagagagatcttccctgacatcaaagctcgaaggcttggtggccggggccagtccaaatattgctacagtggcataaggaggaagaccttggtgtctatgccacccctgcctggacttgacctaaagggttctgagagtccagaaatgggcccagaagtaaccccagcacctcgagatgaactggtggaggcagcgtgtgccctgacctgtgactgggcagagcggatcctgaaacggtccttcagttccatcgttgaggtcgcccgcttcctgctacagcagcatctcatctctgcccgatctgcacatgcccatgtgcttaaggccatggggcttgctgaagaggacgaacatgcacctcgggaacggtcatctaaaccaaagaatggtttagagaacccagagggtggagcccacaagaagccagagagactggcccagcctcctaaggatctggaagcccgaactggggccggtcctctcgcacgtggagagcggaagaagagtgtagttgagagctcggccccaggagccaataacctgcaggttaatgccctagtggctcggctgcctctgctccttccccgggcccctcgctcactaattccgccaatcccagtctctccacctattctggcccccaggctttcttcaggtgccctgaaagtggctacactgcctctgtctagtagggccggggcacccccagcagctgtgcccatcattaacatgatcttaccaactgttcctgctttgcctggacctggacctgggcctgggcgagctccacctgggggactcactcagccccggggcacagagaacagagaggtaggcataggtggtgaccaaggaccacatgacaagggtgtcaagaggacagctgaagtacctgtgagtgaggccagtgggcaggctccaccagctaaagcagcaaagcaggatatagaggatacagcaagtgatgccaaaaggaaacgggggcgccctcgaaaaaagtcaggtggaagtggggaaaggaattctacccctctcaagtcagcagctgccatggaatctgcccagtcctcaaggttaccatgggagacatggggctcaggaggggaaggcaactcagctggaggggcagagaggccagggccaatgggagaggctgaaaagggggcagtacttgcccagggtcagggagatggtactgtttccaaaggaggaaggggccccggttcccagcataccaaagaagcagaagataaaattcccttggtcccctcaaaagtgagtgtcatcaagggcagcagaagccaaaaggaggcttttcctttggcaaagggagaggtagacactgcaccacagggtaataaagacttaaaggagcatgtgcttcaaagttccttatcccaggagcataaagacccaaaagcaacacccccatgatacaggtctgtggggaagagtgtttatatccctacgttaactttgcctagtagaggcccttctttgcacttgcttctcatttggctattcttttcctaaggaagtccattctcctctgtacagacagctgagtcacccagtctacttagtacctggttgctgcctctgaccttttcagcttgataccctgggctttagtgtaaccaataaatctgtagtgaccttacctgtattccctgtgctatcctgtgggaaggtaggaatgggctaagtatgatgaatatataggttagggatcttttggttttaaatcacagaaaacctaattcaaactggcttaaaataaaaaggatttattggttcatgtaactagaaagtccataggtagtgctggctccaggtgaagacttgacccagtagttcagtatgtctctaaataccggactgacttttttctcactgttgcatcttctgtaggaccatttaagtctgggccacttaatggctgccagcattcctaagattacacttttccccatttatgtccaatcagaaaaagaaggcatctttgtaccagaaatctcagcaaaagccctaatattcacactgattaggcctgggtcacatgtccaccctgaccaatcactgtggccaggaggatgatacatgctaatttgcttattctatatcatggacaacacctttggggaaaagggtgggggtcagcctccccaaaatcacatggattccccaagtggaaactaggagcagggagttgcttgggtggccgctaacaccaggctactcttattttagcttgctaagttgagatcagctagacctgctttcttttctcctcagtcttgcatttccctcaatacaagctgtagcctctttcctcgtttctagtctcagaaggaaggagagggaagccattctcctctagggactcttcagtctcatttagatgatagtccctttttttctacctccatattagagatggagctccttccttttcctgtttcttaatttttgtcttctcattcctgcttccctctcaccctattgccagttccaccaactagagtgaaagacttcctagccatttcattaaatctattctgtatccaccaggtggcagcatcttgtcatacgtgtcaggacttaggactgcggggtttaggttagatgtcacggaaaaagctagttctgtggtcaggcggcaccaatgagaaaggaatgcagaccctccagatgtatccttgggaaaagcagtaaaccaactaatatttattgaagacctactttgtcctctacatagggtagcttctgtcagggaatcttggttcttcccaagaaacactgattttctttcagggagacttcatgtgttcatttatttccaccacagcagattttaagaaattataatatgtaatatttgatatctataaagagtatatctaacgtgaataaattatgaagcatactaatgagtacctatgacccataacacatatacattaaaacattttaaatacca

SEQ ID NO: 29 - protein, 616 aa, Homo sapiens
MAEDEPDAKSPKTGGRAPPGGAEAGEPTTLLQRLRGTISKAVQNKVEGILQDVQKFSDNDKLYLYLQLPSGPTTGDKSSEPSTLSNEEYMYAYRWIRNHLEEHTDTCLPKQSVYDAYRKYCESLACCRPLSTANFGKIIREIFPDIKARRLGGRGQSKYCYSGIRRKTLVSMPPLPGLDLKGSESPEMGPEVTPAPRDELVEAACALTCDWAERILKRSFSSIVEVARFLLQQHLISARSAHAHVLKAMGLAEEDEHAPRERSSKPKNGLENPEGGAHKKPERLAQPPKDLEARTGAGPLARGERKKSVVESSAPGANNLQVNALVARLPLLLPRAPRSLIPPIPVSPPILAPRLSSGALKVATLPLSSRAGAPPAAVPIINMILPTVPALPGPGPGPGRAPPGGLTQPRGTENREVGIGGDQGPHDKGVKRTAEVPVSEASGQAPPAKAAKQDIEDTASDAKRKRGRPRKKSGGSGERNSTPLKSAAAMESAQSSRLPWETWGSGGEGNSAGGAERPGPMGEAEKGAVLAQGQGDGTVSKGGRGPGSQHTKEAEDKIPLVPSKVSVIKGSRSQKEAFPLAKGEVDTAPQGNKDLKEHVLQSSLSQEHKDPKATPP

SEQ ID NO: 30 - DNA, 3611 bp, Homo sapiens
  source 1..3611: mol_type = genomic DNA
  CDS 215..2065
attcagaaaaaggaaaataaagcaacgcacgagccggctgggccaggaccttggggctgggctggagtgaaggtggctacgagttttccagatttaggagacttcagaaaggtggggcagatagaatggagatggcaaagatctctttgggcatatatgggcctggcgaagtaatggaataatttctaattttcggagaagccctcatgccgggatggcagaagatgagcctgatgctaagagccccaagactgggggaagggcccccccaggtggtgctgaggctggggaacctaccacccttcttcagaggctccgaggtaccatttccaaggccgtgcagaacaaagtagaggggatcctgcaagatgtacagaaattttctgacaatgacaagctgtatctctaccttcagctcccctcaggacccaccactggagacaaaagctcagagccaagtacactgagcaatgaggagtacatgtatgcctataggtggatccgcaaccacctggaagagcacactgacacctgtctgccaaagcaaagtgtttatgatgcctatcggaagtactgtgagagtcttgcctgttgccgcccactcagcacagccaactttggcaagatcatcagagagatcttccctgacatcaaagctcgaaggcttggtggccggggccagtccaaatattgctacagtggcataaggaggaagaccttggtgtctatgccacccctgcctggacttgacctaaagggttctgagagtccagaaatgggcccagaagtaaccccagcacctcgagatgaactggtggaggcagcgtgtgccctgacctgtgactgggcagagcggatcctgaaacggtccttcagttccatcgttgaggtcgcccgcttcctgctacagcagcatctcatctctgcccgatctgcacatgcccatgtgcttaaggccatggggcttgctgaagaggacgaacatgcacctcgggaacggtcatctaaaccaaagaatggtttagagaacccagagggtggagcccacaagaagccagagagactggcccagcctcctaaggatctggaagcccgaactggggccggtcctctcgcacgtggagagcggaagaagagtgtagttgagagctcggccccaggagccaataacctgcaggttaatgccctagtggctcggctgcctctgctccttccccgggcccctcgctcactaattccgccaatcccagtctctccacctattctggcccccaggctttcttcaggtgccctgaaagtggctacactgcctctgtctagtagggccggggcacccccagcagctgtgcccatcattaacatgatcttaccaactgttcctgctttgcctggacctggacctgggcctgggcgagctccacctgggggactcactcagccccggggcacagagaacagagaggtaggcataggtggtgaccaaggaccacatgacaagggtgtcaagaggacagctgaagtacctgtgagtgaggccagtgggcaggctccaccagctaaagcagcaaagcaggatatagaggatacagcaagtgatgccaaaaggaaacgggggcgccctcgaaaaaagtcaggtggaagtggggaaaggaattctacccctctcaagtcagcagctgccatggaatctgcccagtcctcaaggttaccatgggagacatggggctcaggaggggaaggcaactcagctggaggggcagagaggccagggccaatgggagaggctgaaaagggggcagtacttgcccagggtcagggagatggtactgtttccaaaggaggaaggggccccggttcccagcataccaaagaagcagaagataaaattcccttggtcccctcaaaagtgagtgtcatcaagggcagcagaagccaaaaggaggcttttcctttggcaaagggagaggtagacactgcaccacagggtaataaagacttaaaggagcatgtgcttcaaagttccttatcccaggagcataaagacccaaaagcaacacccccatgatacaggtctgtggggaagagtgtttatatccctacgttaactttgcctagtagaggcccttctttgcacttgcttctcatttggctattcttttcctaaggaagtccattctcctctgtacagacagctgagtcacccagtctacttagtacctggttgctgcctctgaccttttcagcttgataccctgggctttagtgtaaccaataaatctgtagtgaccttacctgtattccctgtgctatcctgtgggaaggtaggaatgggctaagtatgatgaatatataggttagggatcttttggttttaaatcacagaaaacctaattcaaactggcttaaaataaaaaggatttattggttcatgtaactagaaagtccataggtagtgctggctccaggtgaagacttgacccagtagttcagtatgtctctaaataccggactgacttttttctcactgttgcatcttctgtaggaccatttaagtctgggccacttaatggctgccagcattcctaagattacacttttccccatttatgtccaatcagaaaaagaaggcatctttgtaccagaaatctcagcaaaagccctaatattcacactgattaggcctgggtcacatgtccaccctgaccaatcactgtggccaggaggatgatacatgctaatttgcttattctatatcatggacaacacctttggggaaaagggtgggggtcagcctccccaaaatcacatggattccccaagtggaaactaggagcagggagttgcttgggtggccgctaacaccaggctactcttattttagcttgctaagttgagatcagctagacctgctttcttttctcctcagtcttgcatttccctcaatacaagctgtagcctctttcctcgtttctagtctcagaaggaaggagagggaagccattctcctctagggactcttcagtctcatttagatgatagtccctttttttctacctccatattagagatggagctccttccttttcctgtttcttaatttttgtcttctcattcctgcttccctctcaccctattgccagttccaccaactagagtgaaagacttcctagccatttcattaaatctattctgtatccaccaggtggcagcatcttgtcatacgtgtcaggacttaggactgcggggtttaggttagatgtcacggaaaaagctagttctgtggtcaggcggcaccaatgagaaaggaatgcagaccctccagatgtatccttgggaaaagcagtaaaccaactaatatttattgaagacctactttgtcctctacatagggtagcttctgtcagggaatcttggttcttcccaagaaacactgattttctttcagggagacttcatgtgttcatttatttccaccacagcagattttaagaaattataatatgtaatatttgatatctataaagagtatatctaacgtgaataaattatgaagcatactaatgagtacctatgacccataacacatatacattaaaacattttaaatacca

SEQ ID NO: 31 - protein, 616 aa, Homo sapiens; identical to SEQ ID NO: 29

SEQ ID NO: 32 - DNA, 2825 bp, Homo sapiens
  source 1..2825: mol_type = genomic DNA
  CDS 157..975
cgcgcaggctgcggtcgcgcaggcgcagtcggggcgccttcccggtataggcgccttttaccccagcgtgtcctgagtctttggttcgcgaagtgccgttaggccaagcaggtgctaaaagcccggggtcgtggaccccggccaggtcttagcagcatggaggcgcagggtgtagcggagggcgcggggccgggcgccgccagcggcgtgccccaccccgcggccctagccccggctgcggctcccaccttggcgccagcctcggtggcggccgcggcctctcaattcaccctgctagtgatgcaaccctgtgctgggcaggacgaggctgcggcccccgggggcagcgttggggcgggcaagcccgttaggtacctgtgcgaaggggccggggatggcgaagaggaggctggggaggacgaggcggacctgttagacacttcggaccctccggggggaggcgagagcgcggctagtttggaggatctagaggacgaggagactcactcggggggcgagggcagcagcgggggcgcccggaggcggggcagcggtgggggcagcatgagcaagacctgcacctacgaaggctgcagcgagaccacgagccaggtggccaagcagcgcaaaccgtggatgtgcaagaaacaccgcaacaagatgtacaaggacaagtataaaaagaagaagagcgaccaggccctgaactgcggtgggactgcctcgactggcagcgcgggaaacgtcaaactcgaggaaagtgcagataacatactctccattgttaaacaaagaacaggatcttttggggatcgtcctgcaagacctactcttttagaacaagtgttaaatcaaaaaagactgtcgttactaagaagtccagaagtagtgcaatttttacagaaacagcaacagctattaaatcagcaagttttggagcaaagacaacagcagtttccaggaacatcaatgtgagggaacttaccaagaacatctacatggttttttatcttattgtaatagatgagcatatttttttaccagacataaatggggtaataatctatgcctgtagaacataaacattttcctgtaaatgtatgtgtgcatttggggataagtaagtattgcactttgtgcatctaatctttcagattactgtgagtttgaagaagtcagcttatctttccaaataacatttaattataatgttttttaaaaaatatattcctcttcagtcattgttactgaaggtaatgaagcagttactttctgtggaagtcataaagttaatagatattaatcttgactcatctagctcagtggttctcatcaagggtcaatttgattgtcatagtgaccttgaaaaccactggcttttagtgagtggccaggaatgctaaatgttctgcagtgtcaggggtagtcccacatactaaagattgtctcacccgcagtgccaataacactcctaagaaatgttgatggctattttgtggtgctaacatgtagttggggcacctataattgggttctcttaataacctttctttgcagttaagactgaagctgtcaaagaggtaagcacattttatatagacgtaaggaaagtgattattgtttaatatctgtgaatttaggatgtgcatctcttttcagaggtgtgttagtaaaacctgacggattaactaagcacactgggatgtgtctcctacagttggcttctctctttgatgttacctgttagtgctgatctcttaaagcagacatttcttgtttgttgaatttgtgaacagtatagatctcagcccaccaatgccaagacaaaattatttttcttatacttattttttattaaacaaaatgaaaaagatccttttcaaaaaggtgatcctgaaaataaaactaacactccagtattttgtcattgtttttcgcaattgagctatctgaaaactgttattcctaagtaatgttcaaaaatgataagtaatctggatacctttttcttatactttctcctaggaaaactttaaaactttaaaaaggcaaacctaccaataggaataacaaattaaatgtcaagagagtatatccaatattaggatataaatgtatgtgtctcaagtttaactctacaaaaatttgttacttgttttttaaactctatatataaagttcgacttaatcatggctgttctaagaagtacttatggagagcaagaacgtttttgttcatttcttaatgtgtgtgtttttacttgcatatctgttcaaaacacttttaacaaaattaattcattaaagtccagttgttgacctttgagttagctgatttctttattctgttctttagtttattcttactagatgcagaggaattcatctactgtctgttattaactgttagtttattctcatacttacgatgttgagagtttttttgaagcttaagttaccctttatggtggaaaacattagcttatgcttctttagatggaataatgggaaaggagggaaatgggaaatggatggaaatgggaaaggagggaaaataatagcccagtgagagctgaatgaaaagggactgaatttaaatatttgtaagaactttgtgatgatgagtaattgtcagacgtgggatagataactgagaggctcagaatctttaccaaggatattttttaggataaggtagctgcctgttcatgaatttggataagaatagtaggacaatattcaacacaatttaatttttgtctgccacattagacatttttttaccttataaaatgatcaataaagcaataaggtttattttgggt

SEQ ID NO: 33 - protein, 272 aa, Homo sapiens
MEAQGVAEGAGPGAASGVPHPAALAPAAAPTLAPASVAAAASQFTLLVMQPCAGQDEAAAPGGSVGAGKPVRYLCEGAGDGEEEAGEDEADLLDTSDPPGGGESAASLEDLEDEETHSGGEGSSGGARRRGSGGGSMSKTCTYEGCSETTSQVAKQRKPWMCKKHRNKMYKDKYKKKKSDQALNCGGTASTGSAGNVKLEESADNILSIVKQRTGSFGDRPARPTLLEQVLNQKRLSLLRSPEVVQFLQKQQQLLNQQVLEQRQQQFPGTSM

SEQ ID NO: 34 - DNA, 4654 bp, Homo sapiens
  source 1..4654: mol_type = genomic DNA
  CDS 134..3526
ggttagtgatgaggctagtgatgaggctgtgtgcttctgagctgggcatccgaaggcatccttggggaagctgagggcacgaggaggggctgccagactccgggagctgctgcctggctgggattcctacacaatgcgttgcctggctccacgccctgctgggtcctacctgtcagagccccaaggcagctcacagtgtgccaccatggagttggggcccctagaaggtggctacctggagcttcttaacagcgatgctgaccccctgtgcctctaccacttctatgaccagatggacctggctggagaagaagagattgagctctactcagaacccgacacagacaccatcaactgcgaccagttcagcaggctgttgtgtgacatggaaggtgatgaagagaccagggaggcttatgccaatatcgcggaactggaccagtatgtcttccaggactcccagctggagggcctgagcaaggacattttcaagcacataggaccagatgaagtgatcggtgagagtatggagatgccagcagaagttgggcagaaaagtcagaaaagacccttcccagaggagcttccggcagacctgaagcactggaagccagctgagccccccactgtggtgactggcagtctcctagtgggaccagtgagcgactgctccaccctgccctgcctgccactgcctgcgctgttcaaccaggagccagcctccggccagatgcgcctggagaaaaccgaccagattcccatgcctttctccagttcctcgttgagctgcctgaatctccctgagggacccatccagtttgtccccaccatctccactctgccccatgggctctggcaaatctctgaggctggaacaggggtctccagtatattcatctaccatggtgaggtgccccaggccagccaagtaccccctcccagtggattcactgtccacggcctcccaacatctccagaccggccaggctccaccagccccttcgctccatcagccactgacctgcccagcatgcctgaacctgccctgacctcccgagcaaacatgacagagcacaagacgtcccccacccaatgcccggcagctggagaggtctccaacaagcttccaaaatggcctgagccggtggagcagttctaccgctcactgcaggacacgtatggtgccgagcccgcaggcccggatggcatcctagtggaggtggatctggtgcaggccaggctggagaggagcagcagcaagagcctggagcgggaactggccaccccggactgggcagaacggcagctggcccaaggaggcctggctgaggtgctgttggctgccaaggagcaccggcggccgcgtgagacacgagtgattgctgtgctgggcaaagctggtcagggcaagagctattgggctggggcagtgagccgggcctgggcttgtggccggcttccccagtacgactttgtcttctctgtcccctgccattgcttgaaccgtccgggggatgcctatggcctgcaggatctgctcttctccctgggcccacagccactcgtggcggccgatgaggttttcagccacatcttgaagagacctgaccgcgttctgctcatcctagacggcttcgaggagctggaagcgcaagatggcttcctgcacagcacgtgcggaccggcaccggcggagccctgctccctccgggggctgctggccggccttttccagaagaagctgctccgaggttgcaccctcctcctcacagcccggccccggggccgcctggtccagagcctgagcaaggccgacgccctatttgagctgtccggcttctccatggagcaggcccaggcatacgtgatgcgctactttgagagctcagggatgacagagcaccaagacagagccctgacgctcctccgggaccggccacttcttctcagtcacagccacagccctactttgtgccgggcagtgtgccagctctcagaggccctgctggagcttggggaggacgccaagctgccctccacgctcacgggactctatgtcggcctgctgggccgtgcagccctcgacagcccccccggggccctggcagagctggccaagctggcctgggagctgggccgcagacatcaaagtaccctacaggaggaccagttcccatccgcagacgtgaggacctgggcgatggccaaaggcttagtccaacacccaccgcgggccgcagagtccgagctggccttccccagcttcctcctgcaatgcttcctgggggccctgtggctggctctgagtggcgaaatcaaggacaaggagctcccgcagtacctagcattgaccccaaggaagaagaggccctatgacaactggctggagggcgtgccacgctttctggctgggctgatcttccagcctcccgcccgctgcctgggagccctactcgggccatcggcggctgcctcggtggacaggaagcagaaggtgcttgcgaggtacctgaagcggctgcagccggggacactgcgggcgcggcagctgctggagctgctgcactgcgcccacgaggccgaggaggctggaatttggcagcacgtggtacaggagctccccggccgcctctcttttctgggcacccgcctcacgcctcctgatgcacatgtactgggcaaggccttggaggcggcgggccaagacttctccctggacctccgcagcactggcatttgcccctctggattggggagcctcgtgggactcagctgtgtcacccgtttcagggctgccttgagcgacacggtggcgctgtgggagtccctgcagcagcatggggagaccaagctacttcaggcagcagaggagaagttcaccatcgagcctttcaaagccaagtccctgaaggatgtggaagacctgggaaagcttgtgcagactcagaggacgagaagttcctcggaagacacagctggggagctccctgctgttcgggacctaaagaaactggagtttgcgctgggccctgtctcaggcccccaggctttccccaaactggtgcggatcctcacggccttttcctccctgcagcatctggacctggatgcgctgagtgagaacaagatcggggacgagggtgtctcgcagctctcagccaccttcccccagctgaagtccttggaaaccctcaatctgtcccagaacaacatcactgacctgggtgcctacaaactcgccgaggccctgccttcgctcgctgcatccctgctcaggctaagcttgtacaataactgcatctgcgacgtgggagccgagagcttggctcgtgtgcttccggacatggtgtccctccgggtgatggacgtccagtacaacaagttcacggctgccggggcccagcagctcgctgccagccttcggaggtgtcctcatgtggagacgctggcgatgtggacgcccaccatcccattcagtgtccaggaacacctgcaacaacaggattcacggatcagcctgagatgatcccagctgtgctctggacaggcatgttctctgaggacactaaccacgctggaccttgaactgggtacttgtggacacagctcttctccaggctgtatcccatgagcctcagcatcctggcacccggcccctgctggttcagggttggcccctgcccggctgcggaatgaaccacatcttgctctgctgacagacacaggcccggctccaggctcctttagcgcccagttgggtggatgcctggtggcagctgcggtccacccaggagccccgaggccttctctgaaggacattgcggacagccacggccaggccagagggagtgacagaggcagccccattctgcctgcccaggcccctgccaccctggggagaaagtacttctttttttttatttttagacagagtctcactgttgcccaggctggcgtgcagtggtgcgatctgggttcactgcaacctccgcctcttgggttcaagcgattcttctgcttcagcctcccgagtagctgggactacaggcacccaccatcatgtctggctaatttttcatttttagtagagacagggttttgccatgttggccaggctggtctcaaactcttgacctcaggtgatccacccacctcagcctcccaaagtgctgggattacaagcgtgagccactgcaccgggccacagagaaagtacttctccaccctgctctccgaccagacaccttgacagggcacaccgggcactcagaagacactgatgggcaacccccagcctgctaattccccagattgcaacaggctgggcttcagtggcagctgcttttgtctatgggactcaatgcactgacattgttggccaaagccaaagctaggcctggccagatgcaccagcccttagcagggaaacagctaatgggacactaatggggcggtgagaggggaacagactggaagcacagcttcatttcctgtgtcttttttcactacattataaatgtctctttaatgtcacaggcaggtccagggtttgagttcataccctgttaccattttggggtacccactgctctggttatctaatatgtaacaagccaccccaaatcatagtggcttaaaacaacactcacattta

SEQ ID NO: 35 - protein, 1130 aa, Homo sapiens
MRCLAPRPAGSYLSEPQGSSQCATMELGPLEGGYLELLNSDADPLCLYHFYDQMDLAGEEEIELYSEPDTDTINCDQFSRLLCDMEGDEETREAYANIAELDQYVFQDSQLEGLSKDIFKHIGPDEVIGESMEMPAEVGQKSQKRPFPEELPADLKHWKPAEPPTVVTGSLLVGPVSDCSTLPCLPLPALFNQEPASGQMRLEKTDQIPMPFSSSSLSCLNLPEGPIQFVPTISTLPHGLWQISEAGTGVSSIFIYHGEVPQASQVPPPSGFTVHGLPTSPDRPGSTSPFAPSATDLPSMPEPALTSRANMTEHKTSPTQCPAAGEVSNKLPKWPEPVEQFYRSLQDTYGAEPAGPDGILVEVDLVQARLERSSSKSLERELATPDWAERQLAQGGLAEVLLAAKEHRRPRETRVIAVLGKAGQGKSYWAGAVSRAWACGRLPQYDFVFSVPCHCLNRPGDAYGLQDLLFSLGPQPLVAADEVFSHILKRPDRVLLILDGFEELEAQDGFLHSTCGPAPAEPCSLRGLLAGLFQKKLLRGCTLLLTARPRGRLVQSLSKADALFELSGFSMEQAQAYVMRYFESSGMTEHQDRALTLLRDRPLLLSHSHSPTLCRAVCQLSEALLELGEDAKLPSTLTGLYVGLLGRAALDSPPGALAELAKLAWELGRRHQSTLQEDQFPSADVRTWAMAKGLVQHPPRAAESELAFPSFLLQCFLGALWLALSGEIKDKELPQYLALTPRKKRPYDNWLEGVPRFLAGLIFQPPARCLGALLGPSAAASVDRKQKVLARYLKRLQPGTLRARQLLELLHCAHEAEEAGIWQHVVQELPGRLSFLGTRLTPPDAHVLGKALEAAGQDFSLDLRSTGICPSGLGSLVGLSCVTRFRAALSDTVALWESLQQHGETKLLQAAEEKFTIEPFKAKSLKDVEDLGKLVQTQRTRSSSEDTAGELPAVRDLKKLEFALGPVSGPQAFPKLVRILTAFSSLQHLDLDALSENKIGDEGVSQLSATFPQLKSLETLNLSQNNITDLGAYKLAEALPSLAASLLRLSLYNNCICDVGAESLARVLPDMVSLRVMDVQYNKFTAAGAQQLAASLRRCPHVETLAMWTPTIPFSVQEHLQQQDSRISLR

SEQ ID NO: 36 - DNA, 1697 bp, Homo sapiens
  source 1..1697: mol_type = genomic DNA
  CDS 108..890
atttccagtgctagaggcccacagtttcagtctcatctgcctccactcggcctcagttcctcatcactgttcctgtgctcacagtcatcaattatagaccccacaacatgcgccctgaagacagaatgttccatatcagagctgtgatcttgagagccctctccttggctttcctgctgagtctccgaggagctggggccatcaaggcggaccatgtgtcaacttatgccgcgtttgtacagacgcatagaccaacaggggagtttatgtttgaatttgatgaagatgagatgttctatgtggatctggacaagaaggagaccgtctggcatctggaggagtttggccaagccttttcctttgaggctcagggcgggctggctaacattgctatattgaacaacaacttgaataccttgatccagcgttccaaccacactcaggccaccaacgatccccctgaggtgaccgtgtttcccaaggagcctgtggagctgggccagcccaacaccctcatctgccacattgacaagttcttcccaccagtgctcaacgtcacgtggctgtgcaacggggagctggtcactgagggtgtcgctgagagcctcttcctgcccagaacagattacagcttccacaagttccattacctgacctttgtgccctcagcagaggacttctatgactgcagggtggagcactggggcttggaccagccgctcctcaagcactgggaggcccaagagccaatccagatgcctgagacaacggagactgtgctctgtgccctgggcctggtgctgggcctagtcggcatcatcgtgggcaccgtcctcatcataaagtctctgcgttctggccatgacccccgggcccaggggaccctgtgaaatactgtaaaggtgacaaaatatctgaacagaagaggacttaggagagatctgaactccagctgccctacaaactccatctcagcttttcttctcacttcatgtgaaaactactccagtggctgactgaattgctgacccttcaagctctgtccttatccattacctcaaagcagtcattccttagtaaagtttccaacaaatagaaattaatgacactttggtagcactaatatggagattatcctttcattgagccttttatcctctgttctcctttgaagaacccctcactgtcaccttcccgagaataccctaagaccaataaatacttcagtatttcagagcggggagactctgagtcattcttactggaagtctaggaccaggtcacatgtgaatactatttcttgaaggtgtggtttcaacctctgttgccgatgtggttactaaaggttctgatcccacttgaacggaaaggtctgaggatattgattcagtcctgggtttttccctaactacaggatagggtggggtagagaaaggatatttgggggaaattttacttggatgaagattttcttggatgtagtttgaagactgcagtgtttgaagtctctgagggaagagatttggtctgtctggatcaagatttcaggcagattaggattccattcacagcccctgagcttccttcccaaggctgtattgtaattatagcaatatttcatggaggatttttctacatgataaactaagagccaagaaataaaatttttaaaatgccctaaaaaaaaaaaaaaaaaa

SEQ ID NO: 37 - protein, 260 aa, Homo sapiens
MRPEDRMFHIRAVILRALSLAFLLSLRGAGAIKADHVSTYAAFVQTHRPTGEFMFEFDEDEMFYVDLDKKETVWHLEEFGQAFSFEAQGGLANIAILNNNLNTLIQRSNHTQATNDPPEVTVFPKEPVELGQPNTLICHIDKFFPPVLNVTWLCNGELVTEGVAESLFLPRTDYSFHKFHYLTFVPSAEDFYDCRVEHWGLDQPLLKHWEAQEPIQMPETTETVLCALGLVLGLVGIIVGTVLIIKSLRSGHDPRAQGTL

SEQ ID NO: 38 - DNA, 777 bp, Homo sapiens
  source 1..777: mol_type = genomic DNA
  CDS 1..777
atgatggttctgcaggtttctgcggccccccggacagtggctctgacggcgttactgatggtgctgctcacatctgtggtccagggcagggccactccagagaattacgtgtaccagggacggcaggaatgctacgcgtttaatgggacacagcgcttcctggagagatacatctacaaccgggaggagtacgcgcgcttcgacagcgacgtgggggagttccgggcggtgacggagctggggcggcctgctgcggagtactggaacagccagaaggacatcctggaggagaagcgggcagtgccggacagggtatgcagacacaactacgagctggacgaggccgtgaccctgcagcgccgagtccagcctaaggtgaacgtttccccctccaagaaggggcccctgcagcaccacaacctgcttgtctgccacgtgacagatttctacccaggcagcattcaagtccgatggttcctgaatggacaggaggaaacagctggggtcgtgtccaccaacctgatccgtaatggagactggaccttccagatcctggtgatgctggaaatgaccccccagcagggagacgtctacatctgccaagtggagcacaccagcctggacagtcctgtcaccgtggagtggaaggcacagtctgattctgcccagagtaagacattgacgggagctgggggcttcgtgctggggctcatcatctgtggagtgggcatcttcatgcacaggaggagcaagaaagttcaacgaggatctgcataa

SEQ ID NO: 39 - protein, 258 aa, Homo sapiens [sequence omitted]

SEQ ID NO: 40 - DNA, 768 bp, Homo sapiens
  source 1..768: mol_type = genomic DNA
  CDS 1..768
atgatcctaaacaaagctctgctgctgggggccctcgctctgaccaccgtgatgagcccctgtggaggtgaagacattgtggctgaccacgttgcctcttgtggtgtaaacttgtaccagttttacggtccctctggccagtacacccatgaatttgatggagatgaggagttctacgtggacctggagaggaaggagactgcctggcggtggcctgagttcagcaaatttggaggttttgacccgcagggtgcactgagaaacatggctgtggcaaaacacaacttgaacatcatgattaaacgctacaactctaccgctgctaccaatgaggttcctgaggtcacagtgttttccaagtctcccgtgacactgggtcagcccaacaccctcatttgtcttgtggacaacatctttcctcctgtggtcaacatcacatggctgagcaatgggcagtcagtcacagaaggtgtttctgagaccagcttcctctccaagagtgatcattccttcttcaagatcagttacctcaccttcctcccttctgctgatgagatttatgactgcaaggtggagcactggggcctggaccagcctcttctgaaacactgggagcctgagattccagcccctatgtcagagctcacagagactgtggtctgcgccctggggttgtctgtgggcctcgtgggcattgtggtgggcactgtcttcatcatccaaggcctgcgttcagttggtgcttccagacaccaagggccattgtga

SEQ ID NO: 41 - protein, 255 aa, Homo sapiens [sequence omitted]

SEQ ID NO: 42 - DNA, 786 bp, Homo sapiens
  source 1..786: mol_type = genomic DNA
  CDS 1..786
atgtcttggaaaaaggctttgcggatccccggaggccttcgggcagcaactgtgaccttgatgctgtcgatgctgagcaccccagtggctgagggcagagactctcccgaggatttcgtgtaccagtttaagggcatgtgctacttcaccaacgggacagagcgcgtgcgtcttgtgagcagaagcatctataaccgagaagagatcgtgcgcttcgacagcgacgtgggggagttccgggcggtgacgctgctggggctgcctgccgccgagtactggaacagccagaaggacatcctggagaggaaacgggcggcggtggacagggtgtgcagacacaactaccagttggagctccgcacgaccttgcagcggcgagtggagcccacagtgaccatctccccatccaggacagaggccctcaaccaccacaacctgctggtctgctcggtgacagatttctatccagcccagatcaaagtccggtggtttcggaatgaccaggaggagacagctggcgttgtgtccaccccccttattaggaatggtgactggaccttccagatcctggtgatgctggaaatgactccccagcgtggagacgtctacacctgccacgtggagcaccccagcctccagagccccatcaccgtggagtggcgggctcaatctgaatctgcccagagcaagatgctgagtggcattggaggcttcgtgctggggctgatcttcctcgggctgggccttatcatccatcacaggagtcagaaagggctcctgcactga

SEQ ID NO: 43 - protein, 261 aa, Homo sapiens
MSWKKALRIPGGLRAATVTLMLSMLSTPVAEGRDSPEDFVYQFKGMCYFTNGTERVRLVSRSIYNREEIVRFDSDVGEFRAVTLLGLPAAEYWNSQKDILERKRAAVDRVCRHNYQLELRTTLQRRVEPTVTISPSRTEALNHHNLLVCSVTDFYPAQIKVRWFRNDQEETAGVVSTPLIRNGDWTFQILVMLEMTPQRGDVYTCHVEHPSLQSPITVEWRAQSESAQSKMLSGIGGFVLGLIFLGLGLIIHHRSQKGLLH

SEQ ID NO: 44 - DNA, 1312 bp, Homo sapiens
  source 1..1312: mol_type = genomic DNA
  CDS 110..874
ttttaatggtcagactctattacaccccacattctcttttcttttattcttgtctgttctgcctcactcccgagctctactgactcccaacagagcgcccaagaagaaaatggccataagtggagtccctgtgctaggatttttcatcatagctgtgctgatgagcgctcaggaatcatgggctatcaaagaagaacatgtgatcatccaggccgagttctatctgaatcctgaccaatcaggcgagtttatgtttgactttgatggtgatgagattttccatgtggatatggcaaagaaggagacggtctggcggcttgaagaatttggacgatttgccagctttgaggctcaaggtgcattggccaacatagctgtggacaaagccaacctggaaatcatgacaaagcgctccaactatactccgatcaccaatgtacctccagaggtaactgtgctcacaaacagccctgtggaactgagagagcccaacgtcctcatctgtttcatagacaagttcaccccaccagtggtcaatgtcacgtggcttcgaaatggaaaacctgtcaccacaggagtgtcagagacagtcttcctgcccagggaagaccaccttttccgcaagttccactatctccccttcctgccctcaactgaggacgtttacgactgcagggtggagcactggggcttggatgagcctcttctcaagcactgggagtttgatgctccaagccctctcccagagactacagagaacgtggtgtgtgccctgggcctgactgtgggtctggtgggcatcattattgggaccatcttcatcatcaagggattgcgcaaaagcaatgcagcagaacgcagggggcctctgtaaggcacatggaggtgatggtgtttcttagagagaagatcactgaagaaacttctgctttaatggctttacaaagctggcaatattacaatccttgacctcagtgaaagcagtcatcttcagcattttccagccctatagccaccccaagtgtggatatgcctcttcgattgctccgtactctaacatctagctggcttccctgtctattgccttttcctgtatctattttcctctatttcctatcattttattatcaccatgcaatgcctctggaataaaacatacaggagtctgtctctgctatggaatgccccatggggcatctcttgtgtacttattgtttaaggtttcctcaaactgtgatttttctgaacacaataaactattttgatgatcttgggtggaaaaaaaaaaaaaaaaaaaaaaaaaaaaaaaaaa

SEQ ID NO: 45 - protein, 254 aa, Homo sapiens
MAISGVPVLGFFIIAVLMSAQESWAIKEEHVIIQAEFYLNPDQSGEFMFDFDGDEIFHVDMAKKETVWRLEEFGRFASFEAQGALANIAVDKANLEIMTKRSNYTPITNVPPEVTVLTNSPVELREPNVLICFIDKFTPPVVNVTWLRNGKPVTTGVSETVFLPREDHLFRKFHYLPFLPSTEDVYDCRVEHWGLDEPLLKHWEFDAPSPLPETTENVVCALGLTVGLVGIIIGTIFIIKGLRKSNAAERRGPL

SEQ ID NO: 46 - DNA, 801 bp, Homo sapiens
  source 1..801: mol_type = genomic DNA
  CDS 1..801
atggtgtgtctgaagctccctggaggctcctgcatgacagcgctgacagtgacactgatggtgctgagctccccactggctttggctggggacacccgaccacgtttcttgtggcagcttaagtttgaatgtcatttcttcaatgggacggagcgggtgcggttgctggaaagatgcatctataaccaagaggagtccgtgcgcttcgacagcgacgtgggggagtaccgggcggtgacggagctggggcggcctgatgccgagtactggaacagccagaaggacctcctggagcagaggcgggccgcggtggacacctactgcagacacaactacggggttggtgagagcttcacagtgcagcggcgagttgagcctaaggtgactgtgtatccttcaaagacccagcccctgcagcaccacaacctcctggtctgctctgtgagtggtttctatccaggcagcattgaagtcaggtggttccggaacggccaggaagagaaggctggggtggtgtccacaggcctgatccagaatggagattggaccttccagaccctggtgatgctggaaacagttcctcggagtggagaggtttacacctgccaagtggagcacccaagtgtgacgagccctctcacagtggaatggagagcacggtctgaatctgcacagagcaagatgctgagtggagtcgggggcttcgtgctgggcctgctcttccttggggccgggctgttcatctacttcaggaatcagaaaggacactctggacttcagccaacaggattcctgagctga

SEQ ID NO: 47 - protein, 266 aa, Homo sapiens
MVCLKLPGGSCMTALTVTLMVLSSPLALAGDTRPRFLWQLKFECHFFNGTERVRLLERCIYNQEESVRFDSDVGEYRAVTELGRPDAEYWNSQKDLLEQRRAAVDTYCRHNYGVGESFTVQRRVEPKVTVYPSKTQPLQHHNLLVCSVSGFYPGSIEVRWFRNGQEEKAGVVSTGLIQNGDWTFQTLVMLETVPRSGEVYTCQVEHPSVTSPLTVEWRARSESAQSKMLSGVGGFVLGLLFLGAGLFIYFRNQKGHSGLQPTGFLS

SEQ ID NO: 48 - DNA, 1122 bp, Homo sapiens
  source 1..1122: mol_type = genomic DNA
  CDS 87..872
gatctaaggccaccctctcggggagggagttggggaagctgggttggctgggttggtagctcctacctactgtgtggcaagaaggtatgggtcatgaacagaaccaaggagctgcgctgctacagatgttaccacttctgtggctgctaccccactcctgggccgtccctgaagctcctactccaatgtggccagatgacctgcaaaaccacacattcctgcacacagtgtactgccaggatgggagtcccagtgtgggactctctgaggcctacgacgaggaccagcttttcttcttcgacttttcccagaacactcgggtgcctcgcctgcccgaatttgctgactgggctcaggaacagggagatgctcctgccattttatttgacaaagagttctgcgagtggatgatccagcaaatagggccaaaacttgatgggaaaatcccggtgtccagagggtttcctatcgctgaagtgttcacgctgaagcccctggagtttggcaagcccaacactttggtctgttttgtcagtaatctcttcccacccatgctgacagtgaactggcagcatcattccgtccctgtggaaggatttgggcctacttttgtctcagctgtcgatggactcagcttccaggccttttcttacttaaacttcacaccagaaccttctgacattttctcctgcattgtgactcacgaaattgaccgctacacagcaattgcctattgggtaccccggaacgcactgccctcagatctgctggagaatgtgctgtgtggcgtggcctttggcctgggtgtgctgggcatcatcgtgggcattgttctcatcatctacttccggaagccttgctcaggtgactgattcttccagaccagagtttgatgccagcagcttcggccatccaaacagaggatgctcagatttctcacatcctgcccaggatctcctcttagggtagaagtctctgggacatccctggggtgtgtgtgtagatttcccacctggggactctgctgtccctgggcttgcatcccagggatcccagagtggcctgcctatcacaaccacatcccttccccccacaaggcaataaatctcatttctttatatc

SEQ ID NO: 49 - protein, 261 aa, Homo sapiens
MGHEQNQGAALLQMLPLLWLLPHSWAVPEAPTPMWPDDLQNHTFLHTVYCQDGSPSVGLSEAYDEDQLFFFDFSQNTRVPRLPEFADWAQEQGDAPAILFDKEFCEWMIQQIGPKLDGKIPVSRGFPIAEVFTLKPLEFGKPNTLVCFVSNLFPPMLTVNWQHHSVPVEGFGPTFVSAVDGLSFQAFSYLNFTPEPSDIFSCIVTHEIDRYTAIAYWVPRNALPSDLLENVLCGVAFGLGVLGIIVGIVLIIYFRKPCSGD

SEQ ID NO: 50 - DNA, 1412 bp, Homo sapiens
  source 1..1412: mol_type = genomic DNA
  CDS 264..1055
atttgtccctgcctacctagccaatctgtccctgtttgggacactggactcccgtgagctggaaggaacagatttaatatctaggggctgggtatccccacatcactcatttggggggtcaagggacccgggcaatatagtattctgctcagtgtctggagatcatctacccaggctggggcttctgggacaggcgaggacccacggaccctggaagagctggtccaggggactgaactcccggcatctttacagagcagagcatgatcacattcctgccgctgctgctggggctcagcctgggctgcacaggagcaggtggcttcgtggcccatgtggaaagcacctgtctgttggatgatgctgggactccaaaggatttcacatactgcatctccttcaacaaggatctgctgacctgctgggatccagaggagaataagatggccccttgcgaatttggggtgctgaatagcttggcgaatgtcctctcacagcacctcaaccaaaaagacaccctgatgcagcgcttgcgcaatgggcttcagaattgtgccacacacacccagcccttctggggatcactgaccaacaggacacggccaccatctgtgcaagtagccaaaaccactccttttaacacgagggagcctgtgatgctggcctgctatgtgtggggcttctatccagcagaagtgactatcacgtggaggaagaacgggaagcttgtcatgcctcacagcagtgcgcacaagactgcccagcccaatggagactggacataccagaccctctcccatttagccttaaccccctcttacggggacacttacacctgtgtggtagagcacactggggctcctgagcccatccttcgggactggacacctgggctgtcccccatgcagaccctgaaggtttctgtgtctgcagtgactctgggcctgggcctcatcatcttctctcttggtgtgatcagctggcggagagctggccactctagttacactcctcttcctgggtccaattattcagaaggatggcacatttcctagaggcagaatcctacaacttccactccaagtgagaaggagattcaaactcaatgatgctaccatgcctctccaacatcttcaaccccctgacattatcttggatcctatggtttctccatccaattctttgaatttcccagtctcccctatgtaaaacttagcaacttgggggacctcattcctgggactatgctgtaaccaaattattgtccaaggctatatttctgggatgaatataatctgaggaagggagttaaagaccctcctggggctctcagtgtgccatagaggacagcaactggtgattgtttcagagaaataaactttggtggaaatattgttaaaaaaaaaaaaaaa

SEQ ID NO: 51 - protein, 263 aa, Homo sapiens
MITFLPLLLGLSLGCTGAGGFVAHVESTCLLDDAGTPKDFTYCISFNKDLLTCWDPEENKMAPCEFGVLNSLANVLSQHLNQKDTLMQRLRNGLQNCATHTQPFWGSLTNRTRPPSVQVAKTTPFNTREPVMLACYVWGFYPAEVTITWRKNGKLVMPHSSAHKTAQPNGDWTYQTLSHLALTPSYGDTYTCVVEHTGAPEPILRDWTPGLSPMQTLKVSVSAVTLGLGLIIFSLGVISWRRAGHSSYTPLPGSNYSEGWHIS

SEQ ID NO: 52 - DNA, 3485 bp, Homo sapiens
  source 1..3485: mol_type = genomic DNA
  CDS 77..829
cttcttctttacctccgccttgttcctgtcctcaccacacggactgagactgatttgattaaagcaccagagtgtaatggccctcagagcagggctggtcctggggttccacaccctgatgaccctcctgagcccgcaggaggcaggggccaccaaggctgaccacatgggctcctacggacccgccttctaccagtcttacggcgcctcgggccagttcacccatgaatttgatgaggaacagctgttctctgtggacctgaagaaaagcgaggccgtgtggcgtctgcctgagtttggtgactttgcccgctttgacccgcagggcgggctggccggcatcgccgcaatcaaagcccatctggacatcctggtggagcgctccaaccgcagcagagccatcaacgtgcctccacgggtgaccgtgctccccaagtctcgggtggagctgggccagcccaacatcctcatctgcatcgtggacaacatcttcccccctgtgatcaatatcacctggctgcgcaacggccaaactgtcactgagggagtggcccagaccagcttctattcccagcctgaccatttgttccgcaagttccactacctgcccttcgtgccctcagccgaggacgtctatgactgccaggtggagcactggggcctggatgcgccactcctcaggcattgggagctccaggtgcctattccaccaccagatgccatggagaccctggtctgtgccctgggcctggccatcggcctggtgggcttcctcgtgggcaccgtcctcatcatcatgggcacatatgtgtccagtgtccccaggtaatgatccttctgagagaaatgacttgtgggagacaccctgcagatcctcatgggtttgtgacagcccctgcgtgctcagtgccctttaagtgcatcccgctgtgctgactttgagtgggatcaacatctgtcctacgggtcccctcttttttggccccagtattcatggcagggtttgttggacacctactagcttcccttcccattcaacacacacacacattcttgctctacccaaagctctggctggcagcactaaatgctttggtggtgtttgcactgtgtcctttccaggccttggccagttcttccaggggtgaggcatgtggtgctggggattggcagccatcctggggcccacacaggtgtgtcttgctccatttggcccattgtgtgttactttgtgaatgagccatttcacatggacttcatgaaatttgcctcctgagttcaggtttaccctgaaagggatgcagattatcctgttcctcacgaccccctcagctaacaacagttctgaagggtgctgggacaggacaggctcatggggactccactcctgcctgggtttactctgtatgaagaggccactggtatcctgccatgatgttatctcctttttctacttttccctagagtcccatgcatgataaagagaggcccaaggcttggataaggtggccacttccctcagtggagtcagtcatgttaggtaggaggtggtagagtcggtctgcgaggtatctcgtaagaggggaggtccacctagacacactctaaatatgtggcctagaagattttggtctacttttctgtgaacagaatttaaaacatacaaagagataaatcaccataccacatagtttatgtcaggaccaaaatgagcaatacagattacggttttcaaaccagaatgcacataagaactgcttgggatccttttaaaagtacaggcattggcctggtgcagtggctcattcctgtaatcccagcactttgggaggccaaggggacaggactgcttgaggccaagaggtggaaaccatcttgggctacatagagagaccccatctctacaaagaaagatttaaaaattaaccaggcatggtggctcgcacctgtattcccagccactggggaggctgaggccggaggagtgcttgagcccaggagttcaaggctgcagtgagccaagattgcgccactgcactccagcctaggtgacagagtgagaccctgtctctaaataaataaataaataaaatataaaaataacagtcatcacccagacctactgaattagaatctcgggagtgcagggggcagcaacagggaggctgtcttttctgagatggggtctcactctgtcaccaggctggagtgccatggcatgatctcagctcactgcaacctccacctcctgagttcaagccattctcctgcctcagcctcctgagtagctgggactacaggtgtgcgccactacactcagctaatttttgtattttaagtagagacggggtttcatcatgttggccaggatggcctccatctcttgacctcgtgatccacccaccttccctcccaaagtactggaattacaggcattagccactgtgcccagccgaggctgtcatttttaaccggctctggatgactctgatgcagccatcctggaccttggctgtggtctggtaactggaacccagtgacgtaatcaggtgccatcgggggtcatgggaaagggggatccccaaggtctgaggtggactaggaaggctttctgaagaacctgggtctgttagggcatcagccaatcaaggtacaagtaaatagaggcaaaatgagggtttgaactgtgagcagttggtcctggaaaagaaagaaaccaagagattatggggactcaatgggcttcttaagagagaataagttgaaatcaatgaccagaagaccctgatggaagtggaggagaatcatctcaggcaaactttttgtgtgccagtaacagaaaccctctttgtgtgatcacatgcaaagtataggatatttgcaatatagccatggggaggagtgcagggcccaagggtagattttagccaggcctcccaggaacagaactcggatccgaaaagcccagagaagctagagctgcccctccaacactctcggatccacatggtctgtgttctctagacccccctgcatgttagcggtgttctctctctgtggactgactgtccttctcagtgaacatgtccacccgacagctcctgagtttatatcatctcaaccctcacaacccacagaggctgtgtctcctagtcacagctttaaattactggaaaaataaatgactggccaaacttggagcaggtgtccatcccagccctgtgtagttagagcaggaatcaagatctcaacacaaatgtggctgccaagcactcagccccggggcgaggggtcaagttcttctcagagaaagaggaataagttggttctcagaagacatcacaagatacgtgtgtacccaacaatctctgatctctgctgatcttttgcttagacgttaacttgatgcatcattggaaaggtgtttctctcatctctgtcctaaggcttgataaagtcattaaaattgtgttcttttgactaaa

SEQ ID NO: 53 - protein, 250 aa, Homo sapiens
MALRAGLVLGFHTLMTLLSPQEAGATKADHMGSYGPAFYQSYGASGQFTHEFDEEQLFSVDLKKSEAVWRLPEFGDFARFDPQGGLAGIAAIKAHLDILVERSNRSRAINVPPRVTVLPKSRVELGQPNILICIVDNIFPPVINITWLRNGQTVTEGVAQTSFYSQPDHLFRKFHYLPFVPSAEDVYDCQVEHWGLDAPLLRHWELQVPIPPPDAMETLVCALGLAIGLVGFLVGTVLIIMGTYVSSVPR

SEQ ID NO: 54 - DNA, 1388 bp, Homo sapiens
  source 1..1388: mol_type = genomic DNA
  CDS 98..919
gatttatactcttaatgggtactttctgactgaattttatgagctcattctgaagaggctgacgattttactatctcatttttttcctttctccagaatgggttctgggtgggtcccctgggtggtggctctgctagtgaatctgacccgactggattcctccatgactcaaggcacagactctccagaagattttgtgattcaggcaaaggctgactgttacttcaccaacgggacagaaaaggtgcagtttgtggtcagattcatctttaacttggaggagtatgtacgtttcgacagtgatgtggggatgtttgtggcattgaccaagctggggcagccagatgctgagcagtggaacagccggctggatctcttggagaggagcagacaggccgtggatggggtctgtagacacaactacaggctgggcgcacccttcactgtggggagaaaagtgcaaccagaggtgacagtgtacccagagaggaccccactcctgcaccagcataatctgctgcactgctctgtgacaggcttctatccaggggatatcaagatcaagtggttcctgaatgggcaggaggagagagctggggtcatgtccactggccctatcaggaatggagactggacctttcagactgtggtgatgctagaaatgactcctgaacttggacatgtctacacctgccttgtcgatcactccagcctgctgagccctgtttctgtggagtggagagctcagtctgaatattcttggagaaagatgctgagtggcattgcagccttcctacttgggctaatcttccttctggtgggaatcgtcatccagctaagggctcagaaaggatatgtgaggacgcagatgtctggtaatgaggtctcaagagctgttctgctccctcagtcatgctaaggtcctcactgaagcttctctctctggagcctgaagtagtgatgagtagtctgggccctgggtgaggtaaaggacattcatgaggtcaatgttctgggaataactctcttccctgatccttggaggagcccgaactgattctggagctctgtgttctgagatcatgcatctcccacccatctgcccttctcccttctacgtgtacatcattaatccccattgccaagggcattgtccagaaactcccctgagaccttactccttccagccccaaatcatttacttttctgtggtccagccctactcctataagtcatgatctccaaagctttctgtcttccaactgcagtctccacagtcttcagaagacaaatgctcaggtagtcactgtttccttttcactgtttttaaaaaccttttattgtcaaataaaatggagatacaaaaaatgtaaaaaaaaaaaaaaaaa

SEQ ID NO: 55 - protein, 273 aa, Homo sapiens
MGSGWVPWVVALLVNLTRLDSSMTQGTDSPEDFVIQAKADCYFTNGTEKVQFVVRFIFNLEEYVRFDSDVGMFVALTKLGQPDAEQWNSRLDLLERSRQAVDGVCRHNYRLGAPFTVGRKVQPEVTVYPERTPLLHQHNLLHCSVTGFYPGDIKIKWFLNGQEERAGVMSTGPIRNGDWTFQTVVMLEMTPELGHVYTCLVDHSSLLSPVSVEWRAQSEYSWRKMLSGIAAFLLGLIFLLVGIVIQLRAQKGYVRTQMSGNEVSRAVLLPQSC

SEQ ID NO: 56 - DNA, 4507 bp, synthetic construct
  misc_feature 1..4507: note = Synthetic
  source 1..4507: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcggtcgagtatcaagttgctttctgtcccggcagaggaagccagatcgctgagggtccggtctccagtttgcctcctgctatatccattggaagagaaaagtttgtgacttgggcccccaagttttgagagaactgggctttcggcgcggggggacagaggaggctcgtggggaggtgagtggacctcctggatcgatttaggaggctcgggtgggactgggaccaatcgcacctttgttgagccgtcactcattatggggagcccctcccaacaaccatagcgaatatttattgagctccacgcttagcagatcccccgataagctatgtacagacataattcgttgtaggtctcatttccacattttgtcgtggaggaaactgaggctcaaaggttaagtcacgtgcccaagttctcacggcctataaattgagggattcgaacccagacagctgaccccagagccccggcttttttttcctttcttttttttttttttttttttttttgagacagagtcttgctctgtcgccccagctacagtgcagtggtgcgatctctgctcaccgcaacctccacctcccgagttcaagcaattctgcctcagcctcccgagtagctgggatcacaggcgcccgccaccgcgcttggctaatttttgtatttttagtacagatggggtttcaccatggtggccagcctggtctcgaactcctgacctcatgatccaccctcctcggcctcccaaagtgctgggattacagccgtgagccactgtgccctgccagagcccaggctcttaaacattgcacattgtaggtgctttggtggtggggaatggtagacctcgaaagcagttggactctggttttcatttccctggctctggtaattaacctggacctcagtttacccaccctcacagtgcaaggctaggtatgcagtcggtgcctactttattataactgttgataattattgtcatctctccccttctgacacctccgccagctttccccatctagataacttcgtataatgtatgctatacgaagttatggatccatcgattctgcgatcgctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttccaccatgcccacgctactgcgggtttatatagacggtccccacgggatggggaaaaccaccaccacgcaactgctggtggccctgggttcgcgcgacgatatcgtctacgtacccgagccgatgacttactggcgggtgctgggggcttccgagacaatcgcgaacatctacaccacacaacaccgcctcgaccagggtgagatatcggccggggacgcggcggtggtaatgacaagcgcccagataacaatgggcatgccttatgccgtgaccgacgccgttctggctcctcatatcgggggggaggctgggagctcacatgccccgcccccggccctcaccctcatcttcgaccgccatcccatcgccgccctcctgtgctacccggccgcgcggtaccttatgggcagcatgaccccccaggccgtgctggcgttcgtggccctcatcccgccgaccttgcccggcaccaacatcgtgcttggggcccttccggaggacagacacatcgaccgcctggccaaacgccagcgccccggcgagcggctggacctggctatgctggctgcgattcgccgcgtttacgggctacttgccaatacggtgcggtatctgcagtgcggcgggtcgtggcgggaggactggggacagctttcggggacggccgtgccgccccagggtgccgagccccagagcaacgcgggcccacgaccccatatcggggacacgttatttaccctgtttcgggcccccgagttgctggcccccaacggcgacctgtataacgtgtttgcctgggccttggacgtcttggccaaacgcctccgttccatgcacgtctttatcctggattacgaccaatcgcccgccggctgccgggacgccctgctgcaacttacctccgggatggtccagacccacgtcaccacccccggctccataccgacgatatgcgacctggcgcgcacgtttgcccgggagatgggatcggccattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagggtctctagcataacttcgtataatgtatgctatacgaagttataataaaagatccttattttcattggatctgtgtgttggttttttgtgtgggatccgttgactgagtcgacggagcttacccagcctgcagaagacctcatccagacccagcagacccctgcctcagaacttggggaccctgaagaccccggagaggaggctgcagatggctcagacactgtggtcctcagtctctttccctgcacccctgagcctgtgaatcctgaaccggatgccagtgtttcctctccacagggtaggatacctcctctgggattagccctctgggattccatgatgatggaatgtcaggcctcacatggaacctgtgtcttgctttcgtttttgttttgttttgttttgttgttttttgagatggagttttgctcttgttgcccatgctggagtgcaatggcgtgatctcagctcactgcaacctctgcctcctgggttcaactgattctcctgtctcagcctcctgagtagctgggattacaggcttccgccactacgcccagctaattttttgggtatttttagtagagacggggtttcaccatgttggccaggctggtcttgaactcctgacctcaggtgatccgcccacctcggcctcccaaagtgctgggattacaggcatgagccactgtgcccggcctgtttttgttttttgatacagggtctcgctctgttactaaggctggagtgcagtgatgcaatcatagttcactgcagcctcaacttcctgggctgaagccatcactcagcctccctaggagctgggactataggcatgcaaccacacctggctaattttttttttttttttgggaggctggtctccacctcctgggctcaagtgatctgcaggcctccacttccgaaagtgctgggattacaggtgtgagccacagtgcccggcccttcacacctgtttttaactctttgagggtagatagctaggagtgaaattactgggtcagatggtcatcctgttttacttactgaggaactgaggatttatttttgattggagtccaagaatatgcctaatttaggcggccgatcactagtgaattcgcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcag